(19) United States
(12) Patent Application Publication
(10) Pub. No.: US 2026/0041702 A1
(43) Pub. Date: Feb. 12, 2026

(54) AGENTS THAT INHIBIT CCN LIGAND-INDUCED SIGNALLING FOR TREATING DISEASE

(71) Applicant: TRIBUNE THERAPEUTICS AB, Stockholm (SE)

(72) Inventor: Ole Jørgen Kaasbøll, Stockholm (SE)

(21) Appl. No.: 19/104,277

(22) PCT Filed: Aug. 17, 2023
(86) PCT No.: PCT/EP2023/072683

§ 371 Date: Feb. 17, 2025

(30) Foreign Application Priority Data
Aug. 18, 2022 (GB) 2212077.8

(51) Int. Cl. A61K 31/711; A61K 38/17; C12N 15/113; G01N 33/50; G01N 33/68

(52) U.S. Cl. CPC A61K 31/711; A61K 38/177; C12N 15/1138; G01N 33/5023; G01N 33/6845; C12N 2310/14; C12N 2320/31

(57) ABSTRACT

The invention is based in part on the identification of PTK7 as a signalling receptor for C-terminal (domains III-IV) CCN2, and in part on the finding that GPC-1 and GPC-4 may act (individually or in combination) as coreceptors for CCN ligands. The invention relates to the therapeutic use of agents that bind to PTK7 and have PTK7 antagonist activity, agents that inhibit expression of PTK7, agents comprising a PTK polypeptide or a fragment thereof, agents that bind to a GPC coreceptor for CCN ligand and thus inhibit formation of GPC-CCN ligand-PTK7 complexes, agents that inhibit expression of GPCs. or agents comprising a GPC polypeptide or fragment thereof. The invention also relates to pharmaceutical compositions comprising such agents, and screening methods for their identification.

This application claims priority from GB application number 2212077.8 filed on 18 Aug. 2022, the contents and elements of which are herein incorporated by reference for all purposes.

### FIELD OF THE INVENTION

The present invention relates to agents that inhibit CCN ligand-induced signalling for use in a method of treatment or prophylaxis of disease. In particular, the invention relates to but is not limited to an agent that binds to PTK7 and has PTK7 antagonist activity, an agent that inhibits expression of PTK7, an agent comprising a PTK polypeptide or fragment, an agent that binds to a GPC (a coreceptor for CCN ligand) and inhibits GPC-CCN ligand-PTK7 complex formation, an agent that inhibits expression of a GPC, or an agent comprising a GPC polypeptide or fragment for use in therapy. The invention also relates to pharmaceutical compositions and methods for screening for the agents described herein.

### BACKGROUND

CCN proteins are a family of extracellular proteins that are associated with the extracellular matrix (ECM). The CCN denomination is both an abbreviation for Cellular Communication Network factors, and acronym for the original names of the first three members: Cyr61 (Cysteine Rich Angiogenic Inducer 61); CTGF (Connective Tissue Growth Factor) and NOV (nephroblastoma overexpressed) (Perbal B., J Cell Commun Signal. 2018 December; 12 (4): 625-629). The CCN family comprises CCN1 (previously known as Cyr61), CCN2 (previously known as CTGF), CCN3 (previously known as NOV), CCN4 (previously known as WNT1 inducible signalling pathway protein 1/WISP1), CCN5 (previously known as WNT1 inducible signalling pathway protein 2/WISP2) and CCN6 (previously known as WNT1 inducible signalling pathway protein 6/WISP6).

The expression of both CCN2 and the other CCN-family proteins are largely confined to the embryonic state in healthy organisms. However, the CCN proteins are often reactivated during disease states, in particular wound healing processes, fibrosis, and carcinomas (Bradham, D M et al., J Cell Biol 114, 1285-1294 (1991)). In being extracellular proteins mechanistically involved in the development of fibrosis and of limited expression in healthy organisms, CCN proteins appear as attractive therapeutic targets. CCN1-4 and CCN6 are composed of four structural homology domains, while CCN5 is atypical in lacking the fourth domain. CCN5 has previously been reported to oppose the actions of CCN2 and display anti-fibrotic activities (Jeong D et al., JACC VOL. 67, NO. 13, 2016; Xu H. et al., Clin Exp Pharmacol Physiol. 2015 November; 42 (11): 1207-19; Zhang L et al., Int J Mol Med. 2014 February; 33 (2): 478-86; Yoon P O, et al. J Mol Cell Cardiol 2010).

The term homology domains refer to the slight similarity of the amino acid sequences of the conserved domains to the amino acid sequences of domains in other proteins. In the scientific literature CCN proteins are often denominated as modular proteins. The modular protein paradigm entails that the different homology domains function as separate entities that are each capable of eliciting certain biologic activities independent of their tertiary structure (Brigstock, D. R. Endocr Rev 20, 189-206 (1999)). The evidence supporting a modular action hypothesis is generally lacking and only supported by few studies that suffer from technical challenges, e.g., due to unreliable, poorly characterized reagents or lack of reproduction.

In a paper published by Kaasbøll et al. (J. Biol. Chem., 293 (46): 17953-17970, 2018), it was shown that a carboxyl-terminal fragment of the CCN proteins contain their activity and contrary to the scant evidence supporting the modular hypothesis (discussed above) there are multiple reports supporting that a carboxyterminal fragment of CCN2 may be sufficient to recapitulate the activities of CCN2. The first reports supporting this notion were from the Brigstock laboratory, which demonstrated that fragments of domains III-IV (Ball, D. K. et al., Reproduction (2003) 125, 271-284, Ball, D. K. et al., Biol Reprod 59, 828-835 (1998)) or domain IV (Ball, D. K. et al., J Endocrinol 176, R1-7 (2003), Steffen, C. L. et al. Growth Factors 15, 199-213 (1998)), recapitulated the mitogenic activities of full-length CCN2.

The finding that a C-terminal fragment (domains III-IV) of CCN2 is sufficient to recapitulate the activities ascribed to full-length CCN2 was also reported by Mokalled et al. (Science 354, 630-634 (2016)). In the report by Mokalled et al., genetically engineered models were utilized to demonstrate that the carboxyl-terminal domains III-IV of CCN2, but not the amino-terminal domains I-II, recapitulated the effects of full-length CCN2, and affected enhanced regeneration in a zebrafish spinal injury model.

This finding (i.e., that a C-terminal fragment (domains III-IV) of CCN2 is sufficient for activity) was also reproduced by Kaasbøll et al. (J. Biol. Chem., 293 (46): 17953-17970, 2018), who also reported that a C-terminal fragment (domains III-IV) of CCN1 and CCN3 were sufficient to elicit similar signaling and cell physiologic responses.

In the report by Kaasbøll et al. (J. Biol. Chem., 293 (46): 17953-17970, 2018), data was also presented that demonstrate that proteolytic cleavage of CCN2 is necessary to release the active carboxyl-terminal fragment (domains III-IV) and that this fragment can elicit rapid cell signaling responses. Thus, the data suggests that the biologic activity is contained in a C-terminal fragment (domains III-IV), and the N-terminal domains serve as autoinhibitory latency domains that can be released upon proteolytic activity of the “hinge”-region.

The paradigm of secreted CCN proteins being proproteins in which a fragment contains the biologic activity as opposed to the modular paradigm wherein individual domains act independent of one another is analogous to e.g., TGFβ, another pro-fibrotic extracellular matrix-associated protein.

The ability of C-terminal CCN fragments to elicit rapid signalling responses implies an expectation that there is a receptor/receptor complex which should contain at least one component that is necessary for the transmittance of the signalling response. Multiple proteins have been suggested as being receptors responsible for transmitting CCN-signalling responses (Lau L F, J. Cell Commun. Signal. (2016) 10:121-127), however, the reports generally lack key data to substantiate the claims, i.e., well-characterized reagents, ligand-binding experiments combined with evidence of necessity of the putative receptors for rapid signalling responses. Therefore, a mechanistic paradigm supported by data for how CCN proteins elicit their cellular signalling responses is not available so far. Of the multiple suggested receptors for CCN proteins, the most complete evaluation has been for the suggestion of LRP6 as a receptor for CCN2 in a paper published by Johnson et al. (JASN, 28:1769-1782, 2017). In the report by Johnson et al., both experiments with siRNA knock-down of LRP6 combined with short-term CCN2-stimulated phosphoprotein responses and CCN2-LRP6 ligand-binding experiments were presented. However, the CCN2 protein preparation was an E-coli derived domain IV peptide that lacked characterization. Furthermore, Johnson et al., found that the LRP6 binding to the CCN2 domain IV peptide was low and that the CCN2 domain IV peptide did not display increased binding to cells in which LRP6 was overexpressed. Therefore, LRP6 is not demonstrated to be a signalling receptor for CCN2. Accordingly, there remains the need to identify the receptor/receptor complex which is involved in four domain CCN signalling.

### SUMMARY OF THE INVENTION

The present inventors have identified that PTK7 is a signalling receptor for C-terminal (domains III-IV) CCN2. The inventors have also found that C-terminal CCN2 binds to each of GPC-1 and GPC-4, which are each anticipated to act as a coreceptor (individually or in combination) for CCN ligands. Thus, without wishing to be bound by theory, it is believed that the effects of CCN proteins may be mediated by formation of a complex between a GPC (e.g., GPC1 or GPC4), a CCN ligand, and a PTK7 receptor. The finding that CCN ligand-induced signalling occurs via the formation of this complex opens up new possibilities for treating diseases in which CCN ligand-induced signalling has been implicated (e.g., fibrosis) by targeting PTK7 and/or any of the GPC family members.

Accordingly, a first aspect the invention provides an agent that binds to PTK7 and has PTK7 antagonist activity for use in a method of treatment or prophylaxis of fibrosis, metabolic disease, inflammatory disease, autoimmune disease, retinal disease, muscular dystrophy, cardiac disease, or polycystic kidney disease. The agent may inhibit CCN ligand-induced signalling.

In some embodiments, the agent having PTK7 antagonist activity inhibits CCN ligand binding to PTK7. In some embodiments, the CCN ligand comprises or consists of domain IV of a CCN protein, or a fragment thereof. In some embodiments, the CCN ligand comprises or consists of domains III and IV of a CCN protein (also known as “d34CCN”), or a fragment thereof. In some embodiments, the CCN ligand comprises or consists of domains I to IV of a CCN protein, or a fragment thereof. In some embodiments, the CCN protein is CCN2.

Agents that bind to PTK7 and have PTK7 antagonist activity include but are not limited to: (i) agents that inhibit CCN ligand binding to PTK7; (ii) agents that inhibit PTK7 signalling (i.e., PTK-7 dependent signalling transduction); (iii) agents that inhibit homodimerization and/or heterodimerisation of PTK7; (iv) agents that inhibit PTK7 associating with/binding to a coreceptor, e.g., a GPC; and/or (v) agents that promote PTK7 internalisation, optionally agents that promote PTK7 internalisation and degradation. In some embodiments, the agent that binds to PTK7 and has PTK7 antagonist activity may induce homodimerization of PTK7 and/or promote PTK7 internalisation.

The agent of the invention may be selected from the group consisting of an antibody, an antibody-like molecule, an aptamer, and a bifunctional molecule comprising a PTK7 binding moiety and a cell-surface lysosome shuttling receptor binding moiety. In some embodiments, the PTK7 binding portion of the conjugate is an antibody, an antibody-like molecule, an aptamer, or a CCN ligand.

Another aspect of the invention provides an agent that inhibits expression of PTK7 for use in a method of treatment or prophylaxis of fibrosis, metabolic disease, inflammatory disease, autoimmune disease, retinal disease, muscular dystrophy, cardiac disease, or polycystic kidney disease. The agent may inhibit CCN ligand-induced signalling.

The agent described herein may be selected from the group consisting of an agent that inhibits transcription of the gene encoding PTK7, an agent that inhibits post-transcriptional processing of RNA encoding PTK7, an agent that destabilises RNA encoding PTK7, and an agent that promotes the degradation of RNA encoding PTK7.

In some embodiments, the agent is (i) an siRNA, (ii) a shRNA, (iii) an miRNA, (iv) an antisense oligonucleotide (ASO), or (v) an RNA-guided endonuclease system.

The invention further provides an agent comprising a PTK7 polypeptide or fragment thereof for use in a method of treatment or prophylaxis of fibrosis, metabolic disease, inflammatory disease, autoimmune disease, retinal disease, muscular dystrophy, cardiac disease, or polycystic kidney disease, where said PTK7 polypeptide or fragment thereof binds to a CCN ligand and inhibits binding of said CCN ligand to a transmembrane PTK7 receptor and/or a membrane bound GPC. These agents are typically known as “ligand traps”, so called because they trap a ligand (in this case a CCN ligand) and inhibit binding to its receptor and/or coreceptors.

In some embodiments, the CCN ligand comprises or consists of domain IV of a CCN protein, or a fragment thereof. In some embodiments, the CCN ligand comprises or consists of domains III and IV of a CCN protein (e.g., C-terminal fragment), or a fragment thereof. In some embodiments, the CCN ligand comprises or consists of domains I to IV of a CCN protein, or a fragment thereof. In some embodiments, the CCN protein is CCN2.

In some embodiments, the GPC is selected from the group consisting of GPC1, GPC2, GPC3, GPC4, GPC5, and GPC6. In some embodiments, the GPC is GPC1 or GPC4.

The agent may comprise a heterologous moiety. The heterologous moiety is typically a peptide or polypeptide. In some embodiments, the heterologous moiety is a non-PTK7 peptide or polypeptide sequence. In some embodiments, where the heterologous moiety is a peptide or polypeptide, said peptide or polypeptide is a fusion protein with said PTK7 polypeptide or fragment thereof. In some embodiments, the heterologous moiety is an Fc domain, such as a monomeric Fc domain. In other embodiments, the heterologous moiety is a chemical modification, such as PEGylation. The heterologous moiety typically increases the stability of the agent and/or increases the serum half-life of the agent.

Another aspect of the invention provides a method of screening for an agent that binds to PTK7 and has PTK7 antagonist activity, where the agent inhibits CCN ligand-induced signalling. The method comprises the steps of:
(i) providing a PTK7 or a fragment thereof;
(ii) contacting said PTK7 or fragment thereof with one or more candidate agents:
(iii) determining whether the one or more candidate agents bind to said PTK7 or fragment thereof;
(iv) selecting a candidate agent that binds to said PTK7 or fragment thereof from said one or more candidate agents;
(v) providing a cell expressing PTK7;
(vi) contacting said cell with the candidate agent that binds to a PTK7 or fragment thereof; and
(vii) determining whether the candidate agent inhibits CCN ligand-induced signalling.

Step (iii) can be carried out by using binding assays to determine whether one or more candidate agents bind to PTK7. Example assays may include surface plasmon resonance (SPR) or thermal shift assay.

Step (vii) may be carried out by measuring the amount of pAkt (phosphorylated AKT) and/or pERK1/2 (phosphorylated Extracellular signal-regulated kinase) in the cell. In some embodiments, the amount of pAkt and/or pERK1/2 is reduced in the presence of an agent capable of inhibiting CCN ligand-induced signalling.

In some embodiments, step (vi) may further comprise additionally contacting said cell with a CCN ligand. In some embodiments, the CCN ligand comprises or consists of domain IV of a CCN protein, or a fragment thereof. In some embodiments, the CCN ligand comprises or consists of domains III and IV of a CCN protein, or a fragment thereof. In some embodiments, the CCN ligand comprises or consists of domains I to IV of a CCN protein, or a fragment thereof. In some embodiments, the CCN protein is CCN2.

A further aspect of the invention provides a method of screening for an agent that inhibits expression of PTK7, where said agent inhibits CCN ligand-induced signalling. The method comprises the steps of:
(i) providing a cell expressing PTK7;
(ii) contacting said cell with one or more candidate agents;
(iii) determining whether said one or more candidate agents inhibit expression of PTK7;
(iv) selecting a candidate agent that inhibits expression of PTK7 from said one or more candidate agents;
(v) contacting a cell expressing PTK7 with the candidate agent that inhibits expression of PTK7; and
(vi) determining whether the candidate agent inhibits CCN ligand-induced signalling.

PTK7 expression may be determined by measuring total cell expression or plasma membrane expression only. In some embodiments, PTK7 expression is determined by measuring the surface expression of PTK7 on a cell. In some embodiments, step (vi) is carried out by measuring the amount of pAkt and/or pERK1/2 in the cell. In some embodiments, the amount of pAkt and/or pERK1/2 is reduced in the presence of an agent capable of inhibiting expression of PTK7.

In some embodiments, step (v) may further comprise additionally contacting said cell with a CCN ligand. In some embodiments, the CCN ligand comprises or consists of domain IV of a CCN protein, or a fragment thereof. In some embodiments, the CCN ligand comprises or consists of domains III and IV of a CCN protein, or a fragment thereof. In some embodiments, the CCN ligand comprises or consists of domains I to IV of a CCN protein, or a fragment thereof. In some embodiments, the CCN protein is CCN2.

Another aspect provides an agent that binds to a GPC and inhibits GPC-CCN ligand-PTK7 complex formation for use in a method of treatment or prophylaxis of fibrosis, metabolic disease, inflammatory disease, autoimmune disease, cancer, retinal disease, muscular dystrophy, cardiac disease, or polycystic kidney disease. The agent may inhibit CCN ligand-induced signalling.

In some embodiments, the CCN ligand comprises or consists of domain IV of a CCN protein, or a fragment thereof. In some embodiments, the CCN ligand comprises or consists of domains Ill and IV of a CCN protein, or a fragment thereof. In some embodiments, the CCN ligand comprises or consists of domains I to IV of a CCN protein, or a fragment thereof. In some embodiments, the CCN protein is CCN2.

In some embodiments, the agent binds to a GPC and/or a GPC in a complex comprising a CCN ligand and a GPC. In some embodiments, the agent binds to a GPC and inhibits CCN ligand binding to said GPC. In some embodiments, the agent binds to a GPC and inhibits said GPC from associating with and/or binding to PTK7. The GPC may bind directly to PTK7 or indirectly via a CCN ligand. In some embodiments, the agent inhibits PTK7 signalling. In some embodiments, the agent promotes internalisation and degradation of a GPC.

In some embodiments, the GPC is selected from the group consisting of GPC1, GPC2, GPC3, GPC4, GPC5, and GPC6. In some embodiments, the GPC is GPC1 or GPC4.

The agent of the invention may be selected from the group consisting of an antibody, an antibody-like molecule, an aptamer, and a bifunctional molecule comprising a GPC binding moiety and a cell-surface lysosome shuttling receptor binding moiety. In some embodiments, the GPC binding portion of the conjugate is an antibody, an antibody-like molecule, an aptamer, or a CCN ligand.

An aspect of the invention provides an agent that inhibits expression of a GPC for use in a method of treatment or prophylaxis of fibrosis, metabolic disease, inflammatory disease, autoimmune disease, cancer, retinal disease, muscular dystrophy, cardiac disease, or polycystic kidney disease. The agent may inhibit CCN ligand-induced signalling.

The agent described herein may be selected from the group consisting of an agent that inhibits transcription of the gene encoding a GPC, an agent that inhibits post-transcriptional processing of RNA encoding a GPC, an agent that destabilises RNA encoding a GPC, and an agent that promotes the degradation of RNA encoding a GPC.

In some embodiments, the agent is (i) an siRNA, (ii) a shRNA, (iii) an miRNA, (iv) an ASO, or (v) an RNA-guided endonuclease system.

In some embodiments, the GPC is selected from the group consisting of GPC1, GPC2, GPC3, GPC4, GPC5, and GPC6. In some embodiments, the GPC is GPC1 or GPC4.

An aspect of the invention provides an agent comprising a GPC polypeptide or fragment thereof for use in a method of treatment or prophylaxis of fibrosis, metabolic disease, inflammatory disease, autoimmune disease, cancer, retinal disease, muscular dystrophy, cardiac disease, or polycystic kidney disease, where said GPC polypeptide or fragment thereof binds a CCN ligand and inhibits binding of said CCN ligand to a membrane bound PTK7 receptor and/or a membrane bound GPC.

In some embodiments, the CCN ligand comprises or consists of domain IV of a CCN protein, or a fragment thereof. In some embodiments, the CCN ligand comprises or consists of domains III and IV of a CCN protein, or a fragment thereof. In some embodiments, the CCN ligand comprises or consists of domains I to IV of a CCN protein, or a fragment thereof. In some embodiments, the CCN protein is CCN2.

In some embodiments, the GPC polypeptide or fragment thereof is selected from the group consisting of GPC1, GPC2, GPC3, GPC4, GPC5, and GPC6. In some embodiments, the GPC polypeptide or fragment thereof is GPC1 or GPC4.

The agent may comprise a heterologous moiety. The heterologous moiety is typically a peptide or polypeptide. The heterologous moiety is a non-GPC peptide or polypeptide sequence. In some embodiments, where the heterologous moiety is a peptide or polypeptide, said peptide or polypeptide is a fusion protein with said GPC polypeptide or fragment thereof. In some embodiments, the heterologous moiety is an Fc domain, such as a monomeric Fc domain. In other embodiments, the heterologous moiety is a chemical modification, such as PEGylation. The heterologous moiety typically increases the stability of the agent and/or increases the serum half-life of the agent.

A method of screening for an agent that binds to a GPC and inhibits GPC-CCN ligand-PTK7 complex formation is provided, where the agent inhibits CCN ligand-induced signalling. The method comprises the steps of:
(i) providing a GPC or fragment thereof;
(ii) contacting said GPC or fragment thereof with one or more candidate agents:
(iii) determining whether the one or more candidate agents bind to said GPC or fragment thereof;
(iv) selecting a candidate agent that binds to said GPC or fragment thereof from said one or more candidate agents;
(v) providing a cell expressing a GPC;
(vi) contacting said cell with the candidate agent that binds to a GPC or fragment thereof; and
(vii) determining whether the candidate agent inhibits CCN ligand-induced signalling.

Step (iii) can be carried out by using binding assays to determine whether one or more candidate agents bind to a GPC. Example assays may include surface plasmon resonance (SPR) or thermal shift assay.

Step (vii) may be carried out by measuring the amount of pAkt and/or pERK1/2 in the cell. In some embodiments, the amount of pAkt and/or pERK1/2 is reduced in the presence of an agent capable of inhibiting CCN ligand-induced signalling.

In some embodiments, step (vi) may further comprise additionally contacting said cell with a CCN ligand. In some embodiments, the CCN ligand comprises domain IV of a CCN protein, or a fragment thereof. In some embodiments, the CCN ligand comprises or consists of domains III and IV of a CCN protein, or a fragment thereof. In some embodiments, the CCN ligand comprises domains I to IV of a CCN protein, or a fragment thereof. In some embodiments, the CCN protein is CCN2.

Also provided is a method of screening for an agent that inhibits expression of a GPC, where said agent inhibits CCN ligand-induced signalling. The method comprises the steps of:
(i) providing a cell expressing a GPC;
(ii) contacting said cell with one or more candidate agents;
(iii) determining whether said one or more candidate agents inhibit expression of a GPC;
(iv) selecting a candidate agent that inhibits expression of a GPC from said one or more candidate agents;
(v) contacting a cell expressing a GPC with the candidate agent that inhibits expression of a GPC; and
(vi) determining whether the candidate agent inhibits CCN ligand-induced signalling.

GPC expression may be determined by measuring total cell expression or plasma membrane expression. In some embodiments, expression of a GPC is determined by measuring the surface expression of a GPC on a cell. In some embodiments, step (vi) may be carried out by measuring the amount of pAkt and/or pERK1/2 in the cell. In some embodiments, the amount of pAkt and/or pERK1/2 is reduced in the presence of an agent capable of inhibiting expression of a GPC.

In some embodiments, step (v) may further comprise additionally contacting said cell with a CCN ligand. In some embodiments, the CCN ligand comprises domain IV of a CCN protein, or a fragment thereof. In some embodiments, the CCN ligand is a fragment comprising domains Ill and IV of a CCN protein, or a fragment thereof. In some embodiments, the CCN ligand comprises domains I to IV of a CCN protein, or a fragment thereof. In some embodiments, the CCN protein is CCN2.

For both methods described above, the GPC is selected from the group consisting of GPC1, GPC2, GPC3, GPC4, GPC5, and GPC6. In some embodiments, the GPC is GPC1 or GPC4.

An aspect of the invention provides a pharmaceutical composition comprising the agent that binds to PTK7 and has PTK7 antagonist activity as described herein, the agent that inhibits expression of PTK7 as described herein, the agent comprising a PTK7 polypeptide or fragment as described herein, the agent that binds to a GPC and inhibits GPC-CCN ligand-PTK7 complex formation as described herein, the agent that inhibits expression of a GPC as described herein, or the agent comprising a GPC polypeptide or fragment thereof as described herein,
for use in a method of treatment or prophylaxis of fibrosis, metabolic disease, inflammatory disease, autoimmune disease, cancer, retinal disease, muscular dystrophy, cardiac disease, or polycystic kidney disease.

In some embodiments, the pharmaceutical composition comprises two or more agents selected from the group consisting of the agent that binds to PTK7 and has PTK7 antagonist activity as described herein, the agent that inhibits expression of PTK7 as described herein, the agent comprising a PTK7 polypeptide or fragment as described herein, the agent that binds to a GPC and inhibits GPC-CCN ligand-PTK7 complex formation as described herein, the agent that inhibits expression of a GPC as described herein, or the agent comprising a GPC polypeptide or fragment thereof as described herein.

The pharmaceutical composition disclosed herein may comprise (i) the agent that binds to PTK7 and has PTK7 antagonist activity as described herein, the agent that inhibits expression of PTK7 as described herein, the agent comprising a PTK7 polypeptide or fragment as described herein, the agent that binds to a GPC and inhibits GPC-CCN ligand-PTK7 complex formation as described herein, the agent that inhibits expression of a GPC as described herein, or the agent comprising a GPC polypeptide or fragment thereof as described herein; and (ii) at least one other therapeutic agent.

In some embodiments, the at least one other therapeutic agent is selected from the group consisting of an anti-diabetic agent, an anti-obesity agent, an anti-atherogenic agent, a retinopathy treatment, an immunotherapeutic agent, a cell therapy directed towards a target other than PTK7, GPC1, GPC2, GPC3, GPC4, GPC5, or GPC6, and a cancer vaccine directed to a target other than PTK, GPC1, GPC2. GPC3, GPC4, GPC5, or GPC6.

The invention includes the combination of the aspects and preferred features described except where such a combination is clearly impermissible or expressly avoided.

The agents described above are for use in a method of treatment or prophylaxis of fibrosis, metabolic disease, inflammatory disease, autoimmune disease, cancer, retinal disease, muscular dystrophy, cardiac disease, or polycystic kidney disease.

In some embodiments, the metabolic disease is diabetes, such as Type 1 diabetes or Type 2 diabetes. Type 1 diabetes may also be an autoimmune disease as described herein.

In some embodiments, the inflammatory disease or autoimmune disease is selected from the group consisting of rheumatoid arthritis or inflammatory bowel disease (IBD). The two most common forms of IBD comprise ulcerative colitis (UC) or Crohn's disease.

In some embodiments, the cancer is selected from the group consisting of pancreatic cancer, breast cancer, prostate cancer, cervical cancer, ovarian cancer, liver cancer, bladder cancer, brain cancer, bone cancer, blood cancer, melanoma, stomach cancer, mouth cancer, oesophageal cancer, colorectal cancer, or lung cancer. In some embodiments, the pancreatic cancer is pancreatic ductal adenocarcinoma.

In some embodiments, the retinal disease is diabetic retinopathy or age-related macular degeneration. In some embodiments, the muscular dystrophy is Duchenne Muscular dystrophy (DMD).

In some embodiments, the cardiac disease is heart failure or cardiomyocyte hypertrophy.

### SUMMARY OF THE FIGURES

Embodiments and experiments illustrating the principles of the invention will now be discussed with reference to the accompanying figures.

FIG. 1: Total binding of recombinant, extracellular domains of GPC1, GPC4 and PTK7 genetically fused to His or Fc-tags to immobilized recombinant, human C-terminal (domains III-IV) CCN2. 0.2 μg/well d34CCN2 probed with increasing concentrations of indicated proteins in ELISA format. All proteins are extracellular domains N-terminal to fusion partner, either Fc or His-tag. GPC1-His and PTK7-His were produced in HEK293 cells. GPC4-Fc was produced in CHO cells.

FIG. 2: Specific binding of GPC1 to immobilized recombinant, human C-terminal (domains III-IV) CCN2 (d34CCN2). GPC1-His binding to d34CCN2 2 μg/ml (0.2 μg/well). GPC1-His was produced in HEK293 cells.

FIG. 3: Ligand-trap-phosphoprotein assay: Rat2 fibroblasts stimulated with CCN2+/−PTK7-Fc for 15 mins. PTK7-Fc ligand trap reduces recombinant, human C-terminal (domains III-IV) CCN2 (d34CCN2)-stimulated pAKT levels in Rat2 fibroblasts. Molar excess refers to molar quantity of PTK7-Fc relative to that of d34CCN2. PTK7-Fc was produced in HEK293 cells.

FIG. 4: Ligand-trap-phosphoprotein assay: Rat2 fibroblasts stimulated 15 minutes with d34CCN2+/−PTK7-Fc, GPC1-His PTK7-Fc and GPC1 ligand traps reduce recombinant, human C-terminal (domains III-IV) CCN2 (d34CCN2)-stimulated pAKT S473 levels in Rat2 fibroblasts. Molar excess refers to molar quantity of PTK7-Fc or GPC1-His relative to that of d34CCN2. PTK7-Fc was produced in HEK293 cells. GPC1-His was produced in NSO cells.

FIG. 5: PTK-7 siRNA: siRNA towards PTK-7 were effective in reducing the mRNA levels of PTK7 relative to scrambled control at 24 hours, 48 hours and 72 hours following transfection as assessed by TaqMan qPCR.

FIG. 6: siRNA toward PTK7 increases binding of recombinant human C-terminal (domains III-IV) CCN2 coupled to the TriCEPS v.2.0 (TriCEPS-d34CCN2) to the surface to Rat2 fibroblasts as shown by increase in Median Fluorescence intensity (MFI)).

FIG. 7: siRNA toward PTK7 increases binding of recombinant human C-terminal (domains III-IV) CCN2 coupled to the TriCEPS v.2.0 (TriCEPS-d34CCN2) to the surface to Rat2 fibroblasts at 72 hours and for the cells transfected with PTK7_07 an increasing trend can be observed with the time since transfection as shown by an increase in the Median Fluorescence intensity (MFI).

FIG. 8: siRNAs toward PTK7 decreases rapid signalling responses to recombinant, human C-terminal (domains III-IV) CCN2 (d34CCN2) in Rat2 cells as assayed by pAKT S473/GAPDH levels relative to scrambled (control) siRNA. Symbols are mean and error bars are SD.

FIG. 9: siRNAs toward PTK7 decreases rapid signalling responses to recombinant, human C-terminal (domains III-IV) CCN2 (d34CCN2) in Rat2 cells as assayed by pERK ½/total ERK levels relative to cells transfected with non-targeting siRNA, transfection reagent only and non-treated cells. Concentrations of d34CCN2 are indicated on the X-axis. Symbols are mean and error bars are SD.

FIG. 10: siRNAs toward PTK7 reduces the expression of PTK7 mRNA. Columns are mean and error bars are SD.

FIG. 11: siRNAs toward PTK7 have limited impact on cell viability compared to non-targeting control treated cells. Columns are mean and error bars are SD.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present invention is based on the finding that PTK7 is a receptor for CCN ligands (e.g., four domain CCN proteins). The inventors have also surprisingly found that GPCs act as coreceptors for CCN ligands. Without wishing to be bound by theory, CCN ligand-induced signalling is believed to occur by formation of a complex comprising a PTK7 receptor, a GPC, and a CCN ligand. Four domain CCN proteins have been implicated in numerous diseases including but not limited to fibrosis, metabolic disease, inflammatory disease, autoimmune disease, cancer, retinal disease, muscular dystrophy, cardiac disease, and polycystic kidney disease. Accordingly, the finding that the effects of four domain CCN proteins are mediated by the formation of a complex comprising PTK7 receptor, a GPC, and a CCN ligand provides new therapeutic targets for the treatment of diseases in which CCN ligand-induced signalling has been implicated.

Several strategies can be used to inhibit CCN-ligand induced signalling in a subject, which are discussed in detail below.

### PTK7

Protein tyrosine kinase 7 (PTK7), also known as colon carcinoma kinase 4 (CCK4), is a receptor protein tyrosine kinase that has been previously shown to have a role in non-canonical Wnt signalling. PTK7 comprises an extracellular domain comprising seven immunoglobulin-type loops (“lg loop”), a transmembrane domain, and a tyrosine kinase (TK) domain.

PTK7 is a member of the receptor tyrosine kinase (RTK) family. Ligand binding to RTKs typically induces receptor dimerization (for example, homodimerization or heterodimerization), bringing together the tyrosine kinase domains in an active conformation. Ligand binding may also induce clustering or oligomerisation with other PTK7 molecules or with RTK-like receptors. Despite having a tyrosine kinase domain, PTK7 has been found to lack detectable catalytic tyrosine kinase activity. However, previous studies suggest that PTK7 comprises signal transduction activity. For example, it has previously been reported that PTK7 may up-regulate the Akt, ERK, FAK, and c-Jun pathways (Shin et al., Int J Mol Sci, 2022, 23 (4): 2391). It has also been reported that PTK7 interacts with catalytically active RTKs. For example, Shin et al., FASEB J, 2019. 33 (11)) describes PTK7 activation of FGFGR1.

In this specification, “PTK7 signalling” or “PTK7-dependent signalling transduction” is the PTK7-mediated intracellular activity caused by CCN ligand (e.g., domains I to IV of a CCN protein or a fragment comprising domains III and IV of a four domain CCN protein) binding to PTK7. For example, the PTK7-mediated intracellular activity caused by CCN ligand-induced signalling in fibrosis. In a particular example, the term means PTK7-mediated intracellular activity resulting from domains III&IV of CCN1, CCN2, CCN3, CCN4, or CCN6 binding to PTK7. Phosphorylation of Akt or ERK1/2 can be stimulated by CCN ligand e.g., domains III&IV of CCN1, CCN2, CCN3, CCN4, or CCN6, binding to PTK7. Thus, PTK7 signalling may be detected by measuring phosphorylation of Akt and/or phosphorylation of ERK1/2. For example, pAkt S473 can be measured.

Human PTK7, UniProt accession no. Q13308-1
(SEQ ID NO: 1):
MGAARGSPARPRRLPLLSVLLLPLLGGTQTAIVFIKQPSSQDALQGRRAL
LRCEVEAPGPVHVYWLLDGAPVQDTERRFAQGSSLSFAAVDRLQDSGTFQ
CVARDDVTGEEARSANASFNIKWIEAGPVVLKHPASEAEIQPQTQVTLRC
HIDGHPRPTYQWFRDGTPLSDGQSNHTVSSKERNLTLRPAGPEHSGLYSC
CAHSAFGQACSSQNFTLSIADESFARVVLAPQDVVVARYEEAMFHCQFSA
QPPPSLQWLFEDETPITNRSRPPHLRRATVFANGSLLLTQVRPRNAGIYR
CIGQGQRGPPIILEATLHLAEIEDMPLFEPRVFTAGSEERVTCLPPKGLP
EPSVWWEHAGVRLPTHGRVYQKGHELVLANIAESDAGVYTCHAANLAGQR
RQDVNITVATVPSWLKKPQDSQLEEGKPGYLDCLTQATPKPTVVWYRNQM
LISEDSRFEVFKNGTLRINSVEVYDGTWYRCMSSTPAGSIEAQARVQVLE
KLKFTPPPQPQQCMEFDKEATVPCSATGREKPTIKWERADGSSLPEWVTD
NAGTLHFARVTRDDAGNYTCIASNGPQGQIRAHVQLTVAVFITFKVEPER
TTVYQGHTALLQCEAQGDPKPLIQWKGKDRILDPTKLGPRMHIFQNGSLV
IHDVAPEDSGRYTCIAGNSCNIKHTEAPLYVVDKPVPEESEGPGSPPPYK
MIQTIGLSVGAAVAYIIAVLGLMFYCKKRCKAKRLQKQPEGEEPEMECLN
GGPLQNGQPSAEIQEEVALTSLGSGPAATNKRHSTSDKMHFPRSSLQPIT
TLGKSEFGEVFLAKAQGLEEGVAETLVLVKSLQSKDEQQQLDFRRELEMF
GKLNHANVVRLLGLCREAEPHYMVLEYVDLGDLKQFLRISKSKDEKLKSQ
PLSTKQKVALCTQVALGMEHLSNNRFVHKDLAARNCLVSAQRQVKVSALG
LSKDVYNSEYYHFRQAWVPLRWMSPEAILEGDFSTKSDVWAFGVLMWEVF
THGEMPHGGQADDEVLADLQAGKARLPQPEGCPSKLYRLMQRCWALSPKD
RPSFSEIASALGDSTVDSKP

In this specification, “PTK7” refers to PTK7 from any species and includes isoforms, fragments, variants, or homologues of an PTK7 from any species. In particular, “PTK7” or “PTK7 receptor” refers to a PTK7 polypeptide or fragment thereof. In preferred embodiments the species is human (Homo sapiens). Isoforms, fragments, variants, or homologues of PTK7 may optionally be characterised as having at least 70%, preferably one of at least 80%, 85%, 90%, 91%, 92%, 93%, 94%, 95%, 96%, 97%, 98%, 99% or 100% amino acid sequence identity to the amino acid sequence of immature or mature PTK7 from a given species, e.g., human PTK7 (UniProt accession no. Q13308-1, referred herein as SEQ ID NO: 1, and extracellular portion, referred herein as SEQ ID NO: 2).

Extracellular portion of human PTK7, UniProt
accession no. Q13308-1 (SEQ ID NO: 2):
MGAARGSPARPRRLPLLSVLLLPLLGGTQTAIVFIKQPSSQDALQGRRAL
LRCEVEAPGPVHVYWLLDGAPVQDTERRFAQGSSLSFAAVDRLQDSGTFQ
CVARDDVTGEEARSANASFNIKWIEAGPVVLKHPASEAEIQPQTQVTLRC
HIDGHPRPTYQWFRDGTPLSDGQSNHTVSSKERNLTLRPAGPEHSGLYSC
CAHSAFGQACSSQNFTLSIADESFARVVLAPQDVVVARYEEAMFHCQFSA
QPPPSLQWLFEDETPITNRSRPPHLRRATVFANGSLLLTQVRPRNAGIYR
CIGQGQRGPPIILEATLHLAEIEDMPLFEPRVFTAGSEERVTCLPPKGLP
EPSVWWEHAGVRLPTHGRVYQKGHELVLANIAESDAGVYTCHAANLAGQR
RQDVNITVATVPSWLKKPQDSQLEEGKPGYLDCLTQATPKPTVVWYRNQM
LISEDSRFEVFKNGTLRINSVEVYDGTWYRCMSSTPAGSIEAQARVQVLE
KLKFTPPPQPQQCMEFDKEATVPCSATGREKPTIKWERADGSSLPEWVTD
NAGTLHFARVTRDDAGNYTCIASNGPQGQIRAHVQLTVAVFITFKVEPER
TTVYQGHTALLQCEAQGDPKPLIQWKGKDRILDPTKLGPRMHIFQNGSLV
IHDVAPEDSGRYTCIAGNSCNIKHTEAPLYVVDKPVPEESEGPGSPPPYK
MIQT
The N-terminal signal peptide (also known as
“leader peptide”) is typically cleaved off in
mature PTK7 (Q13308-1). Mature PTK7 sequence
has the following sequence (without signal
peptide; SEQ ID NO: 23):
AIVFIKQPSSQDALQGRRALLRCEVEAPGPVHVYWLLDGAPVQDTERRFA
QGSSLSFAAVDRLQDSGTFQCVARDDVTGEEARSANASFNIKWIEAGPVV
LKHPASEAEIQPQTQVTLRCHIDGHPRPTYQWFRDGTPLSDGQSNHTVSS
KERNLTLRPAGPEHSGLYSCCAHSAFGQACSSQNFTLSIADESFARVVLA
PQDVVVARYEEAMFHCQFSAQPPPSLQWLFEDETPITNRSRPPHLRRATV
FANGSLLLTQVRPRNAGIYRCIGQGQRGPPIILEATLHLAEIEDMPLFEP
RVFTAGSEERVTCLPPKGLPEPSVWWEHAGVRLPTHGRVYQKGHELVLAN
IAESDAGVYTCHAANLAGQRRQDVNITVATVPSWLKKPQDSQLEEGKPGY
LDCLTQATPKPTVVWYRNQMLISEDSRFEVFKNGTLRINSVEVYDGTWYR
CMSSTPAGSIEAQARVQVLEKLKFTPPPQPQQCMEFDKEATVPCSATGRE
KPTIKWERADGSSLPEWVTDNAGTLHFARVTRDDAGNYTCIASNGPQGQI
RAHVQLTVAVFITFKVEPERTTVYQGHTALLQCEAQGDPKPLIQWKGKDR
ILDPTKLGPRMHIFQNGSLVIHDVAPEDSGRYTCIAGNSCNIKHTEAPLY
VVDKPVPEESEGPGSPPPYKMIQTIGLSVGAAVAYIIAVLGLMFYCKKRC
KAKRLQKQPEGEEPEMECLNGGPLQNGQPSAEIQEEVALTSLGSGPAATN
KRHSTSDKMHFPRSSLQPITTLGKSEFGEVFLAKAQGLEEGVAETLVLVK
SLQSKDEQQQLDFRRELEMFGKLNHANVVRLLGLCREAEPHYMVLEYVDL
GDLKQFLRISKSKDEKLKSQPLSTKQKVALCTQVALGMEHLSNNRFVHKD
LAARNCLVSAQRQVKVSALGLSKDVYNSEYYHFRQAWVPLRWMSPEAILE
GDFSTKSDVWAFGVLMWEVFTHGEMPHGGQADDEVLADLQAGKARLPQPE
GCPSKLYRLMQRCWALSPKDRPSFSEIASALGDSTVDSKP
Extracellular portion of PTK7 without signal
peptide (SEQ ID NO: 18):
AIVFIKQPSSQDALQGRRALLRCEVEAPGPVHVYWLLDGAPVQDTERRFA
QGSSLSFAAVDRLQDSGTFQCVARDDVTGEEARSANASFNIKWIEAGPVV
LKHPASEAEIQPQTQVTLRCHIDGHPRPTYQWFRDGTPLSDGQSNHTVSS
KERNLTLRPAGPEHSGLYSCCAHSAFGQACSSQNFTLSIADESFARVVLA
PQDVVVARYEEAMFHCQFSAQPPPSLQWLFEDETPITNRSRPPHLRRATV
FANGSLLLTQVRPRNAGIYRCIGQGQRGPPIILEATLHLAEIEDMPLFEP
RVFTAGSEERVTCLPPKGLPEPSVWWEHAGVRLPTHGRVYQKGHELVLAN
IAESDAGVYTCHAANLAGQRRQDVNITVATVPSWLKKPQDSQLEEGKPGY
LDCLTQATPKPTVVWYRNQMLISEDSRFEVFKNGTLRINSVEVYDGTWYR
CMSSTPAGSIEAQARVQVLEKLKFTPPPQPQQCMEFDKEATVPCSATGRE
KPTIKWERADGSSLPEWVTDNAGTLHFARVTRDDAGNYTCIASNGPQGQI
RAHVQLTVAVFITFKVEPERTTVYQGHTALLQCEAQGDPKPLIQWKGKDR
ILDPTKLGPRMHIFQNGSLVIHDVAPEDSGRYTCIAGNSCNIKHTEAPLY
VVDKPVPEESEGPGSPPPYKMIQT

### GPCs

Glypicans (“GPC”) are heparan sulphate proteoglycans that are bound to the external surface of the plasma membrane by a glycosyl-phosphatidylinositol anchor. Heparan sulphate proteoglycans (HSGPs) are cell-surface or extracellular matrix glycoproteins that are modified by the addition of one or more heparan sulphate (HS) chains, a type of glycosaminoglycan (GAG) chain. The HS chains of HSPGs are typically formed by a long linear backbone of repeating disaccharide units of D-glucosamine and uronic acid (D-glucuronic and L-iduronic acids) that can variably be N- and O-sulphated. The major HSPGs are the transmembrane syndecans and the glycosylphosphatidylinositol (GPI) anchored glypicans. A GPI anchor is a posttranslational modification that typically anchors the modified protein in the outer surface of the cell membrane.

The mammalian GPC family of proteins comprise six members, GPC1 through to GPC6. GPC1-6 all comprise a core protein approximately 60 to 70 kDa in size. Glypicans can be bound to the outer surface of the plasma membrane by a glycosylphosphatidylinositol (GPI) anchor. They comprise a hydrophobic domain at the C-terminus of the protein to allow for the addition of the GPI anchor. Typically, within 50 amino acids of this GPI anchor, the HS chains are attached.

The present inventors have found that GPC1 and GPC4 may each (individually or in combination) act as a coreceptor for CCN ligand. Since there are a number of similarities across the GPC family of proteins, it is also anticipated that the other members of the GPC family (i.e., GPC2, GPC3, GPC5 and GPC6) may also act as a coreceptor for a CCN ligand. Accordingly, in this specification, a “GPC” is any one of GPC1, GPC2, GPC3, GPC4, GPC5, or GPC6, or a fragment thereof. Typically, the GPC is membrane bound, with the exception of the ligand traps.

Human GPC1, Uniprot accession no. P35052-1,
(SEQ ID NO: 3):
MELRARGWWLLCAAAALVACARGDPASKSRSCGEVRQIYGAKGFSLSD
VPQAEISGEHLRICPQGYTCCTSEMEENLANRSHAELETALRDSSRVL
QAMLATQLRSFDDHFQHLLNDSERTLQATFPGAFGELYTQNARAFRDL
YSELRLYYRGANLHLEETLAEFWARLLERLFKQLHPQLLLPDDYLDCL
GKQAEALRPFGEAPRELRLRATRAFVAARSFVQGLGVASDVVRKVAQV
PLGPECSRAVMKLVYCAHCLGVPGARPCPDYCRNVLKGCLANQADLDA
EWRNLLDSMVLITDKFWGTSGVESVIGSVHTWLAEAINALQDNRDTLT
AKVIQGCGNPKVNPQGPGPEEKRRRGKLAPRERPPSGTLEKLVSEAKA
QLRDVQDFWISLPGTLCSEKMALSTASDDRCWNGMARGRYLPEVMGDG
LANQINNPEVEVDITKPDMTIRQQIMQLKIMTNRLRSAYNGNDVDFQD
ASDDGSGSGSGDGCLDDLCSRKVSRKSSSSRTPLTHALPGLSEQEGQK
TSAASCPQPPTFLLPLLLFLALTVARPRWR

The human GPC1 gene on chromosome 2q37 encodes the GPC1 protein, consisting of a 558-amino-acid core protein and three predicted HS chains attached at S486, S488, and S490. GPC1 has both a membrane-anchored form and a secreted soluble form.

In this specification, “GPC1” refers to GPC1 from any species and includes isoforms, fragments, variants, or homologues of an GPC1 from any species. In particular, “GPC1” refers to a GPC1 polypeptide or fragment thereof. In preferred embodiments the species is human (Homo sapiens). Isoforms, fragments, variants, or homologues of GPC1 may optionally be characterised as having at least 70%, preferably one of 80%, 85%, 90%, 91%, 92%, 93%, 94%, 95%, 96%, 97%, 98%, 99%, or 100% amino acid sequence identity to the amino acid sequence of immature or mature GPC1 from a given species, e.g., human GPC1 (UniProt accession no. P35052-1; SEQ ID NO: 3).

The signal peptide and propeptide of GPC1 may be
cleaved off in mature GPC1 (P35052-1). Mature
GPC1 may have the following sequence (SEQ ID
NO: 24):
DPASKSRSCGEVRQIYGAKGFSLSDVPQAEISGEHLRICPQGYTCCTS
EMEENLANRSHAELETALRDSSRVLQAMLATQLRSFDDHFQHLLNDSE
RTLQATFPGAFGELYTQNARAFRDLYSELRLYYRGANLHLEETLAEFW
ARLLERLFKQLHPQLLLPDDYLDCLGKQAEALRPFGEAPRELRLRATR
AFVAARSFVQGLGVASDVVRKVAQVPLGPECSRAVMKLVYCAHCLGVP
GARPCPDYCRNVLKGCLANQADLDAEWRNLLDSMVLITDKFWGTSGVE
SVIGSVHTWLAEAINALQDNRDTLTAKVIQGCGNPKVNPQGPGPEEKR
RRGKLAPRERPPSGTLEKLVSEAKAQLRDVQDFWISLPGTLCSEKMAL
STASDDRCWNGMARGRYLPEVMGDGLANQINNPEVEVDITKPDMTIRQ
QIMQLKIMTNRLRSAYNGNDVDFQDASDDGSGSGSGDGCLDDLCSRKV
SRKSSSSRTPLTHALPGLSEQEGQKTS
Human GPC2, UniProt accession no. Q8N158
(SEQ ID NO: 4):
MSALRPLLLLLLPLCPGPGPGPGSEAKVTRSCAETRQVLGARGYSLNL
IPPALISGEHLRVCPQEYTCCSSETEQRLIRETEATFRGLVEDSGSFL
VHTLAARHRKFDEFFLEMLSVAQHSLTQLFSHSYGRLYAQHALIFNGL
FSRLRDFYGESGEGLDDTLADFWAQLLERVFPLLHPQYSFPPDYLLCL
SRLASSTDGSLQPFGDSPRRLRLQITRTLVAARAFVQGLETGRNVVSE
ALKVPVSEGCSQALMRLIGCPLCRGVPSLMPCQGFCLNVVRGCLSSRG
LEPDWGNYLDGLLILADKLQGPFSFELTAESIGVKISEGLMYLQENSA
KVSAQVFQECGPPDPVPARNRRAPPPREEAGRLWSMVTEEERPTTAAG
TNLHRLVWELRERLARMRGFWARLSLTVCGDSRMAADASLEAAPCWTG
AGRGRYLPPVVGGSPAEQVNNPELKVDASGPDVPTRRRRLQLRAATAR
MKTAALGHDLDGQDADEDASGSGGGQQYADDWMAGAVAPPARPPRPPY
PPRRDGSGGKGGGGSARYNQGRSRSGGASIGFHTQTILILSLSALALL
GPR

The human GPC2 gene is located on chromosome 7q22, which encodes the GPC2 protein consisting of a 579-amino-acid core protein and five predicted HS chains at S55, S92, S155, S500, and S502.

In this specification, “GPC2” refers to GPC2 from any species and includes isoforms, fragments, variants, or homologues of an GPC2 from any species. In particular, “GPC2” refers to a GPC2 polypeptide or fragment thereof. In preferred embodiments the species is human (Homo sapiens). Isoforms, fragments, variants, or homologues of GPC2 may optionally be characterised as having at least 70%, preferably one of 80%, 85%, 90%, 91%, 92%, 93%, 94%, 95%, 96%, 97%, 98%, 99%, or 100% amino acid sequence identity to the amino acid sequence of immature or mature GPC2 from a given species, e.g., human GPC2 (UniProt accession number Q8N158; SEQ ID NO: 4).

The signal peptide and propeptide of GPC2 may be
cleaved off in mature GPC2 (Q8N158). Mature GPC2
may have the following sequence (SEQ ID NO: 25):
SEAKVTRSCAETRQVLGARGYSLNLIPPALISGEHLRVCPQEYTCCSS
ETEQRLIRETEATFRGLVEDSGSFLVHTLAARHRKFDEFFLEMLSVAQ
HSLTQLFSHSYGRLYAQHALIFNGLFSRLRDFYGESGEGLDDTLADFW
AQLLERVFPLLHPQYSFPPDYLLCLSRLASSTDGSLQPFGDSPRRLRL
QITRTLVAARAFVQGLETGRNVVSEALKVPVSEGCSQALMRLIGCPLC
RGVPSLMPCQGFCLNVVRGCLSSRGLEPDWGNYLDGLLILADKLQGPF
SFELTAESIGVKISEGLMYLQENSAKVSAQVFQECGPPDPVPARNRRA
PPPREEAGRLWSMVTEEERPTTAAGTNLHRLVWELRERLARMRGFWAR
LSLTVCGDSRMAADASLEAAPCWTGAGRGRYLPPVVGGSPAEQVNNPE
LKVDASGPDVPTRRRRLQLRAATARMKTAALGHDLDGQDADEDASGSG
GGQQYADDWMAGAVAPPARPPRPPYPPRRDGSGGKGGGGSARYNQGRS
RSG
Human GPC3, UniProt accession no. P51654-1
(SEQ ID NO: 5):
MAGTVRTACLVVAMLLSLDFPGQAQPPPPPPDATCHQVRSFFQRLQPG
LKWVPETPVPGSDLQVCLPKGPTCCSRKMEEKYQLTARLNMEQLLQSA
SMELKFLIIQNAAVFQEAFEIVVRHAKNYTNAMFKNNYPSLTPQAFEF
VGEFFTDVSLYILGSDINVDDMVNELFDSLFPVIYTQLMNPGLPDSAL
DINECLRGARRDLKVFGNFPKLIMTQVSKSLQVTRIFLQALNLGIEVI
NTTDHLKFSKDCGRMLTRMWYCSYCQGLMMVKPCGGYCNVVMQGCMAG
VVEIDKYWREYILSLEELVNGMYRIYDMENVLLGLFSTIHDSIQYVQK
NAGKLTTTIGKLCAHSQQRQYRSAYYPEDLFIDKKVLKVAHVEHEETL
SSRRRELIQKLKSFISFYSALPGYICSHSPVAENDTLCWNGQELVERY
SQKAARNGMKNQFNLHELKMKGPEPVVSQIIDKLKHINQLLRTMSMPK
GRVLDKNLDEEGFESGDCGDDEDECIGGSGDGMIKVKNQLRFLAELAY
DLDVDDAPGNSQQATPKDNEISTFHNLGNVHSPLKLLTSMAISVVCFF
FLVH

The human GPC3 gene is on chromosome Xq26 and encodes the canonical GPC3 protein composed of a 580-amino-acid core protein and two predicted HS chains attached at S495 and S509. A non-canonical isoform of GPC3, P51654-3 (SEQ ID NO: 13) is composed of a 603 amino acid core protein.

Non-canonical isoform of GPC3, UniProt accession
no. P51654-3 (SEQ ID NO: 13):
MAGTVRTACLVVAMLLSLDFPGQAQPPPPPPDATCHQVRSFFQRLQPG
LKWVPETPVPGSDLQVCLPKGPTCCSRKMEEKYQLTARLNMEQLLQSA
SMELKFLIIQNAAVFQEAFEIVVRHAKNYTNAMFKNNYPSLTPQAFEF
VGEFFTDVSLYILGSDINVDDMVNELFDSLFPVIYTQLMNPGLPDSAL
DINECLRGARRDLKVFGNFPKLIMTQVSKSLQVTRIFLQALNLGIEVI
NTTDHLKFSKDCGRMLTRMWYCSYCQGLMMVKPCGGYCNVVMQGCMAG
VVEIDKYWREYILSLEELVNGMYRIYDMENVLLGLFSTIHDSIQYVQK
NAGKLTTTETEKKIWHFKYPIFFLCIGLDLQIGKLCAHSQQRQYRSAY
YPEDLFIDKKVLKVAHVEHEETLSSRRRELIQKLKSFISFYSALPGYI
CSHSPVAENDTLCWNGQELVERYSQKAARNGMKNQFNLHELKMKGPEP
VVSQIIDKLKHINQLLRTMSMPKGRVLDKNLDEEGFESGDCGDDEDEC
IGGSGDGMIKVKNQLRFLAELAYDLDVDDAPGNSQQATPKDNEISTFH
NLGNVHSPLKLLTSMAISVVCFFFLVH

In this specification, “GPC3” refers to GPC3 from any species and includes isoforms, fragments, variants, or homologues of an GPC3 from any species. In particular, “GPC3” refers to a GPC3 polypeptide or fragment thereof. In preferred embodiments the species is human (Homo sapiens). Isoforms, fragments, variants, or homologues of GPC3 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature GPC3 from a given species, e.g., human GPC3 (UniProt accession no. P51654-1; SEQ ID NO: 5). GPC3 or fragments thereof may (or may not) be processed into alpha- and beta-subunits as described by De Cat B et al., J Cell Biol. 2003 Nov. 10; 163 (3): 625-35.

The signal peptide and propeptide may be cleaved
off in mature GPC3 (P51654-1). Mature GPC3
(P51654-1) may have the following sequence
(SEQ ID NO: 26):
QPPPPPPDATCHQVRSFFQRLQPGLKWVPETPVPGSDLQVCLPKGPTC
CSRKMEEKYQLTARLNMEQLLQSASMELKFLIIQNAAVFQEAFEIVVR
HAKNYTNAMFKNNYPSLTPQAFEFVGEFFTDVSLYILGSDINVDDMVN
ELFDSLFPVIYTQLMNPGLPDSALDINECLRGARRDLKVFGNFPKLIM
TQVSKSLQVTRIFLQALNLGIEVINTTDHLKFSKDCGRMLTRMWYCSY
CQGLMMVKPCGGYCNVVMQGCMAGVVEIDKYWREYILSLEELVNGMYR
IYDMENVLLGLFSTIHDSIQYVQKNAGKLTTTIGKLCAHSQQRQYRSA
YYPEDLFIDKKVLKVAHVEHEETLSSRRRELIQKLKSFISFYSALPGY
ICSHSPVAENDTLCWNGQELVERYSQKAARNGMKNQFNLHELKMKGPE
PVVSQIIDKLKHINQLLRTMSMPKGRVLDKNLDEEGFESGDCGDDEDE
CIGGSGDGMIKVKNQLRFLAELAYDLDVDDAPGNSQQATPKDNEISTF
HN
Human GPC4, UniProt accession no. O75487-1
(SEQ ID NO: 6):
MARFGLPALLCTLAVLSAALLAAELKSKSCSEVRRLYVSKGFNKNDAP
LHEINGDHLKICPQGSTCCSQEMEEKYSLQSKDDFKSVVSEQCNHLQA
VFASRYKKFDEFFKELLENAEKSLNDMFVKTYGHLYMQNSELFKDLFV
ELKRYYVVGNVNLEEMLNDFWARLLERMFRLVNSQYHFTDEYLECVSK
YTEQLKPFGDVPRKLKLQVTRAFVAARTFAQGLAVAGDVVSKVSVVNP
TAQCTHALLKMIYCSHCRGLVTVKPCYNYCSNIMRGCLANQGDLDFEW
NNFIDAMLMVAERLEGPFNIESVMDPIDVKISDAIMNMQDNSVQVSQK
VFQGCGPPKPLPAGRISRSISESAFSARFRPHHPEERPTTAAGTSLDR
LVTDVKEKLKQAKKFWSSLPSNVCNDERMAAGNGNEDDCWNGKGKSRY
LFAVIGNGLANQGNNPEVQVDTSKPDILILRQIMALRVMTSKMKNAYN
GNDVDFFDISDESSGEGSGSGCEYQQCPSEFDYNATDHAGKSANEKAD
SAGVRPGAQAYLLTVFCILFLVMQREWR

The human GPC4 gene is adjacent to the 3′ end of GPC3 on chromosome Xq26. GPC4 is composed of a 556-amino-acid core protein with the attachment of three HS chains at S494, S498, and S500.

In this specification, “GPC4” refers to GPC4 from any species and includes isoforms, fragments, variants, or homologues of an GPC4 from any species. In particular, “GPC4” refers to a GPC4 polypeptide or fragment thereof. In preferred embodiments the species is human (Homo sapiens). Isoforms, fragments, variants, or homologues of GPC4 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature GPC4 from a given species, e.g., human GPC4 (UniProt accession no. 075487-1; SEQ ID NO: 6).

The signal peptide and propeptide of GPC 4 may
be cleaved off in mature GPC4 (O75487-1). Mature
GPC4 may have the following sequence (SEQ ID
NO: 27):
ALLAAELKSKSCSEVRRLYVSKGFNKNDAPLHEINGDHLKICPQGSTC
CSQEMEEKYSLQSKDDFKSVVSEQCNHLQAVFASRYKKFDEFFKELLE
NAEKSLNDMFVKTYGHLYMQNSELFKDLFVELKRYYVVGNVNLEEMLN
DFWARLLERMFRLVNSQYHFTDEYLECVSKYTEQLKPFGDVPRKLKLQ
VTRAFVAARTFAQGLAVAGDVVSKVSVVNPTAQCTHALLKMIYCSHCR
GLVTVKPCYNYCSNIMRGCLANQGDLDFEWNNFIDAMLMVAERLEGPF
NIESVMDPIDVKISDAIMNMQDNSVQVSQKVFQGCGPPKPLPAGRISR
SISESAFSARFRPHHPEERPTTAAGTSLDRLVTDVKEKLKQAKKFWSS
LPSNVCNDERMAAGNGNEDDCWNGKGKSRYLFAVTGNGLANQGNNPEV
QVDTSKPDILILRQIMALRVMTSKMKNAYNGNDVDFFDISDESSGEGS
GSGCEYQQCPSEFDYNATDHAGKSANEKADS
Human GPC5, UniProt accessio no. P78333 (SEQ ID
NO: 7):
MDAQTWPVGFRCLLLLALVGSARSEGVQTCEEVRKLFQWRLLGAVRGL
PDSPRAGPDLQVCISKKPTCCTRKMEERYQIAARQDMQQFLQTSSSTL
KFLISRNAAAFQETLETLIKQAENYTSILFCSTYRNMALEAAASVQEF
FTDVGLYLFGADVNPEEFVNRFFDSLFPLVYNHLINPGVTDSSLEYSE
CIRMARRDVSPFGNIPQRVMGQMGRSLLPSRTFLQALNLGIEVINTTD
YLHFSKECSRALLKMQYCPHCQGLALTKPCMGYCLNVMRGCLAHMAEL
NPHWHAYIRSLEELSDAMHGTYDIGHVLLNFHLLVNDAVLQAHLNGQK
LLEQVNRICGRPVRTPTQSPRCSFDQSKEKHGMKTTTRNSEETLANRR
KEFINSLRLYRSFYGGLADQLCANELAAADGLPCWNGEDIVKSYTQRV
VGNGIKAQSGNPEVKVKGIDPVINQIIDKLKHVVQLLQGRSPKPDKWE
LLQLGSGGGMVEQVSGDCDDEDGCGGSGSGEVKRTLKITDWMPDDMNF
SDVKQIHQTDTGSTLDTTGAGCAVATESMTFTLISVVMLLPGIW

The human GPC5 gene is located on chromosome 13q32, and encodes the GPC5 protein, which consists of a 572-amino-acid core protein and five predicted HS chains attached at S441, S486, S495, S507 and S509.

In this specification, “GPC5” refers to GPC5 from any species and includes isoforms, fragments, variants, or homologues of an GPC5 from any species. In particular, “GPC5” refers to a GPC5 polypeptide or fragment thereof. In preferred embodiments the species is human (Homo sapiens). Isoforms, fragments, variants, or homologues of GPC5 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature GPC5 from a given species, e.g., human GPC5 (UniProt accession no. P78333; SEQ ID NO: 7).

The signal peptide and propeptide of GPC5 may be
cleaved off in mature GPC5 (P78333). Mature GPC5
may have the following sequence (SEQ ID NO: 28)
(Veugelers et al., Vol 40, Issue 1, 15 Feb. 1997,
pages 24-30):
EGVQTCEEVRKLFQWRLLGAVRGLPDSPRAGPDLQVCISKKPTCCTRK
MEERYQIAARQDMQQFLQTSSSTLKFLISRNAAAFQETLETLIKQAEN
YTSILFCSTYRNMALEAAASVQEFFTDVGLYLFGADVNPEEFVNRFFD
SLFPLVYNHLINPGVTDSSLEYSECIRMARRDVSPFGNIPQRVMGQMG
RSLLPSRTFLQALNLGIEVINTTDYLHFSKECSRALLKMQYCPHCQGL
ALTKPCMGYCLNVMRGCLAHMAELNPHWHAYIRSLEELSDAMHGTYDI
GHVLLNFHLLVNDAVLQAHLNGQKLLEQVNRICGRPVRTPTQSPRCSF
DQSKEKHGMKTTTRNSEETLANRRKEFINSLRLYRSFYGGLADQLCAN
ELAAADGLPCWNGEDIVKSYTQRVVGNGIKAQSGNPEVKVKGIDPVIN
QIIDKLKHVVQLLQGRSPKPDKWELLQLGSGGGMVEQVSGDCDDEDGC
GGSGSGEVKRTLKITDWMPDDMNFSDVKQIHQTDTGSTLDTTGAGCAV
ATES
Human GPC6, UniProt accession no. Q9Y625
(SEQ ID NO: 8):
MPSWIGAVILPLLGLLLSLPAGADVKARSCGEVRQAYGAKGFSLADIP
YQEIAGEHLRICPQEYTCCTTEMEDKLSQQSKLEFENLVEETSHFVRT
TFVSRHKKFDEFFRELLENAEKSLNDMFVRTYGMLYMQNSEVFQDLFT
ELKRYYTGGNVNLEEMLNDFWARLLERMFQLINPQYHFSEDYLECVSK
YTDQLKPFGDVPRKLKIQVTRAFIAARTFVQGLTVGREVANRVSKVSP
TPGCIRALMKMLYCPYCRGLPTVRPCNNYCLNVMKGCLANQADLDTEW
NLFIDAMLLVAERLEGPFNIESVMDPIDVKISEAIMNMQENSMQVSAK
VFQGCGQPKPAPALRSARSAPENFNTRFRPYNPEERPTTAAGTSLDRL
VTDIKEKLKLSKKVWSALPYTICKDESVTAGTSNEEECWNGHSKARYL
PEIMNDGLTNQINNPEVDVDITRPDTFIRQQIMALRVMTNKLKNAYNG
NDVNFQDTSDESSGSGSGSGCMDDVCPTEFEFVTTEAPAVDPDRREVD
SSAAQRGHSLLSWSLTCIVLALQRLCR

The human GPC6 gene is colocalized with GPC5 on chromosome 13q32. It encodes the GPC6 protein composed of a 555-amino-acid core protein and an unknown number of HS chains. GPC6 is most homologous to GPC4 (63%).

In this specification, “GPC6” refers to GPC6 from any species and includes isoforms, fragments, variants, or homologues of an GPC6 from any species. In particular, “GPC6” refers to a GPC6 polypeptide or fragment thereof. In preferred embodiments the species is human (Homo sapiens). Isoforms, fragments, variants, or homologues of GPC6 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature GPC6 from a given species, e.g., human GPC6 (UniProt accession no. Q9Y625; SEQ ID NO: 8).

The signal peptide and propeptide of GPC6 may be
cleaved off in mature GPC6 (Q9Y625). Mature GPC6
may have the following sequence (SEQ ID NO: 29):
DVKARSCGEVRQAYGAKGFSLADIPYQEIAGEHLRICPQEYTCCTTEM
EDKLSQQSKLEFENLVEETSHFVRTTFVSRHKKFDEFFRELLENAEKS
LNDMFVRTYGMLYMQNSEVFQDLFTELKRYYTGGNVNLEEMLNDFWAR
LLERMFQLINPQYHFSEDYLECVSKYTDQLKPFGDVPRKLKIQVTRAF
IAARTFVQGLTVGREVANRVSKVSPTPGCIRALMKMLYCPYCRGLPTV
RPCNNYCLNVMKGCLANQADLDTEWNLFIDAMLLVAERLEGPFNIESV
MDPIDVKISEAIMNMQENSMQVSAKVFQGCGQPKPAPALRSARSAPEN
FNTRFRPYNPEERPTTAAGTSLDRLVTDIKEKLKLSKKVWSALPYTIC
KDESVTAGTSNEEECWNGHSKARYLPEIMNDGLTNQINNPEVDVDITR
PDTFIRQQIMALRVMTNKLKNAYNGNDVNFQDTSDESSGSGSGSGCMD
DVCPTEFEFVTTEAPAVDPDRREVDS

### CCN Ligand

A “CCN ligand” is a ligand derived from a four domain CCN protein, e.g., domains I to IV of a CCN protein or a fragment thereof. The four domain CCN proteins comprise CCN1, CCN2, CCN3, CCN4, and CCN6, and each comprise domains I to IV. The first domain (i.e., “domain l”) shows sequence homologies to insulin-like growth factor binding proteins (IGFBP) and is thus known as IGF-binding protein homology domain. The second domain (i.e., “domain II”) is known as the von Willebrand factor type C repeat (VWC) homology domain, often seen in extracellular matrix (ECM) proteins. The third domain (i.e., “domain III”) is known as the thrombospondin type I (TSP-1) repeat homology domain which has been reported to be involved in the attachment of CCN proteins to integrins. Finally, the fourth domain (i.e., “domain IV”) is a cysteine-rich, C-terminal repeat or cystine knot homology domain. In contrast to CCN1, CCN2, CCN3, CCN4, and CCN6, the fifth member of the family (CCN5) is atypical in lacking domain IV. Accordingly, CCN5 is not a four domain CCN protein.

The CCN ligand may be a fragment of a four domain CCN protein. The fragment may comprise a fragment of a domain, an individual domain, or one or more domains. The CCN ligand may be a fragment of a four domain CCN protein comprising domains III and IV (also referred to as “d34CCN” in this specification).

In some embodiments, the CCN ligand is CCN1. In some embodiments, the CCN ligand is a fragment of CCN1. The fragment of CCN1 may comprise domain IV or domains Ill and IV of CCN1. In some embodiments, the CCN ligand is CCN2. In some embodiments, the CCN ligand is a fragment of CCN2. The fragment of CCN2 may comprise domain IV or domains Ill and IV of CCN2. In some embodiments, the CCN ligand is CCN3. In some embodiments, the CCN ligand is a fragment of CCN3. The fragment of CCN3 may comprise domain IV or domains III and IV of CCN3. In some embodiments, the CCN ligand is CCN4. In some embodiments, the CCN ligand is a fragment of CCN4. The fragment of CCN4 may comprise domain IV or domains III and IV of CCN4. In some embodiments, the CCN ligand is CCN6. In some embodiments, the CCN ligand is a fragment of CCN6. The fragment of CCN6 may comprise domain IV or domains III and IV of CCN6.

“CCN ligand-induced signalling” is the signalling stimulated by a CCN ligand (e.g., a four domain CCN protein or a fragment thereof) binding to a PTK7 receptor. CCN ligand binding to PTK7 (i.e., CCN ligand-induced signalling) is anticipated to result in the phosphorylation of Akt and/or phosphorylation of ERK1/2, for example, pAkt S473 can be measured.

### GPC-CCN Ligand-PTK7 Complex

It is anticipated that one or more of the GPCs act as coreceptors, individually or combination, for a CCN ligand. In this specification, a “coreceptor” is a cell surface protein that participates in a complex with one or more PTK7 receptors and/or one or more CCN ligands.

A “GPC-CCN ligand-PTK7 complex” is a group of three or more associated polypeptides or peptides comprising at least a GPC, a CCN ligand, and a PTK7 protein, or fragments thereof. In some embodiments, the CCN ligand comprises domain IV of a CCN protein, or a fragment thereof. In some embodiments, the CCN ligand comprises domains III and IV of a CCN protein, or a fragment thereof. In some embodiments, the CCN ligand comprises domains I to IV of a CCN protein, or a fragment thereof.

Since ligand binding to RTK-like receptors typically induces receptor homodimerization, the complex may comprise at least two PTK7 proteins. In some embodiments, the complex may comprise two PTK7 polypeptides and two GPC polypeptides. In the case of receptor heterodimerization, PTK7 may form a complex with a different RTK.

A GPC-CCN ligand-PTK7 complex may be formed when one or more of the GPC coreceptors bind one or more of the CCN ligands and associate with one or more PTK7 receptors. For example, the coreceptor may bind the CCN ligand (e.g., d3&4 of CCN protein) and either directly bind to the receptor or bind to the receptor via said CCN ligand. The coreceptor comprising the ligand may associate with the receptor. Formation of the GPC-CCN ligand-PTK7 complex can be determined by measuring CCN ligand-induced signalling (i.e., measuring phosphorylation of Akt and/or phosphorylation of ERK1/2).

The GPC may be selected from the group consisting of GPC1, GPC2, GPC3, GPC4, GPC5, and GPC6. In some embodiments, the GPC may be GPC1 or GPC4. The complex may therefore comprise a single type of GPC (e.g., one or more of GPC1) or the complex may comprise more than one type of GPC (e.g., a complex comprising one or more of GPC1 and/or one or more of GPC4).

### Therapeutic Agents

The present invention relates to therapeutic agents that inhibit CCN ligand-induced signalling for use in therapy (such as fibrosis, metabolic disease, inflammatory disease, autoimmune disease, cancer, retinal disease, muscular dystrophy, cardiac disease, or polycystic kidney disease). The agent described herein may inhibit CCN ligand-induced signalling.

Inhibition of CCN ligand-induced signalling may occur via direct targeting of the PTK7 receptor, by inhibiting expression of the PTK7 receptor, by targeting the coreceptors (e.g., GPCs), or by inhibiting expression of the coreceptors. Alternatively, ligand traps based on PTK7 or GPCs may be used to trap CCN ligands and inhibit CCN ligand-stimulated signalling.

Agents that inhibit CCN ligand-induced signalling via the PTK7 receptor comprise:
agents that target the ligand-receptor interface (i.e., CCN ligand-PTK7 interface);
agents that target the ligand-coreceptor interface (for example, the interface between CCN ligand and GPC1, GPC2, GPC3, GPC4, GPC5, or GPC6);
agents that allosterically bind to PTK7 or the coreceptor (GPC1, GPC2, GPC3, GPC4, GPC5, or GPC6):
agents that inhibit the formation of the coreceptor-ligand-receptor complex; and
ligand traps based on the receptor PTK7 or GPC1, GPC2, GPC3, GPC4, GPC5, or GPC6.

Agents that limit the ability of PTK7 to elicit intracellular signalling events comprise:
agents that orthosterically or allosterically bind to the receptor and thereby limit the function of the receptor or molecules that induce receptor internalisation;
agents that inhibit homodimerization or clustering or oligomerization of PTK7;
agents that inhibit heterodimerization or clustering or oligomerization of PTK7 and other receptor tyrosine kinases (e.g., FGFGR1, Shin et al., FASEB J, 2019. 33 (11));
agents that induce receptor degradation;
agents that proteolytically cleave the extracellular domains of PTK7; and
agents such as sequence-specific RNA-targeting or DNA-targeting approaches that reduce the amount of PTK7 produced.

An aspect of the invention provides an agent that binds to PTK7 and has PTK7 antagonist activity for use in therapy. Also provided is an agent that binds to PTK7 and has PTK7 antagonist activity for use in method of treatment or prophylaxis of fibrosis, metabolic disease, inflammatory disease, autoimmune disease, retinal disease, muscular dystrophy, cardiac disease, or polycystic kidney disease.

In some embodiments, the agent having PTK7 antagonist activity inhibits CCN ligand binding to PTK7. In some embodiments, the agent inhibits PTK7 signalling. The agent may inhibit homodimerization of PTK7 or heterodimerization of PTK7 with other receptor tyrosine kinases. In other embodiments, the agent may promote PTK7 internalisation with or without inducing homodimerization or heterodimerization. In this case, PTK7 is removed from the cell surface. PTK7 may then be sequestered inside the cell. In some embodiments, the internalised PTK7 is degraded (e.g., proteasomal degradation). The agent may bind PTK7 as defined by SEQ ID NO: 1, 2, 18 or 23, or fragments thereof. In some embodiments, the agent binds to the extracellular portion of PTK7. For example, the agent may bind to one or more of the seven immunoglobulin-type loops of PTK7.

An “antagonist” is an agent that inhibits a biological response/activation of the target receptor. In this specification, the term “PTK7 antagonist” refers to any agent including an antibody, antibody fragment, large molecule, or small molecule (less than 10 kD), etc. that is capable of inhibiting the activation or function of PTK7.

In some embodiments, the agent prevents CCN ligand binding to PTK7. In some embodiments, the agent prevents d3&4 of CCN1, CCN2, CCN3, CCN4, and/or CCN6 binding to PTK7. Additionally, or alternatively, the agent may inhibit PTK7 signalling. Inhibition of PTK7 signalling may be determined by measuring the amount of phospho-Akt (pAkt) and/or phospho-ERK1/2 (PERK1/2). It is expected that inhibition of PTK7 signalling inhibits phosphorylation of Akt and/or ERK1/2 (i.e., a reduction in the amount of pAkt and/or pERK1/2).

In some embodiments, the agent prevents PTK7 dimerization (e.g., by preventing ligand binding or by steric hinderance). Despite the lack of a tyrosine kinase domain, it is anticipated that PTK7 may be activated by dimerization (homodimerization) and/or transactivate other receptor tyrosine kinases via heterodimerization. PTK7 may also be activated by clustering or by undergoing oligomerisation (either with other PTK7 molecules or with other RTK-like receptors). Therefore, prevention of dimerization/oligomerisation of receptor tyrosine kinases of which one is PTK7 may also prevent signalling transduction. In some embodiments, the agent may bind to PTK7 and prevent binding to a coreceptor, for example, by steric hinderance. In alternative embodiments, the agent results in the internalisation of PTK7. The internalisation of PTK7 inhibits CCN ligand-stimulated signalling because it removes the receptor from the cell surface, making it unavailable for ligand binding. In some embodiments, the internalised PTK7 is degraded (e.g., via ubiquitination and proteasomal degradation or lysosomal degradation). In some embodiments, the agent promotes PTK7 internalisation and degradation.

The invention provides an agent that inhibits expression of PTK7 for use in therapy. Another aspect of the invention provides an agent comprising a PTK7 polypeptide or fragment thereof for use in therapy. The agent comprising a PTK7 polypeptide or fragment thereof may bind to a CCN ligand and inhibit binding of said CCN ligand to a membrane bound PTK7 receptor and/or a membrane bound GPC.

Also provided are agents that bind to a GPC and inhibit GPC-CCN ligand-PTK7 complex formation for use in therapy. In some embodiments, the agent binds to the GPC and inhibits CCN ligand binding to said GPC (for example, by steric hinderance). In some embodiments, the agent binds to the GPC and inhibits four domain CCN protein binding. In some embodiments, the agent binds to the GPC and inhibits d3&4 of CCN1, CCN2, CCN3, CCN4, and/or CCN6 binding. In some embodiments, the agent binds to a GPC comprising a CCN ligand and inhibits said GPC comprising a CCN ligand from binding to/associating with PTK7 (for example, by steric hinderance). In some embodiments, the agent promotes GPC internalisation and degradation.

Another aspect of the invention provides an agent that inhibits expression of a GPC for use in therapy. Also provided is an agent comprising a GPC or fragment thereof for use in therapy. The agent comprising a GPC or a fragment thereof may bind a CCN ligand and inhibit binding of said CCN ligand to a membrane bound PTK7 receptor and/or a membrane bound GPC.

In some embodiments, the agent may target GPC1, GPC2, GPC3, GPC4, GPC5, and/or GPC6. In some embodiments, agent may target GPC1 and/or GPC4. The agent may bind GPC1 as defined by SEQ ID NO: 3 or 24, GPC2 as defined by SEQ ID NO: 4 or 25, GPC3 as defined by SEQ ID NO: 5, 13, or 26, GPC4 as defined by SEQ ID NO: 6 or 27, GPC5 as defined by SEQ ID NO: 7 or 28, or GPC6 as defined by SEQ ID NO: 8 or 29, or fragments thereof.

### Antibodies

In some embodiments, the agent that binds to PTK7 and has PTK7 antagonist activity is an antibody.

In this specification “antibody” includes a fragment or derivative of an antibody, a synthetic antibody, or a synthetic antibody fragment. In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International RICMP7164916 Biotechnology Symposium Part 2, 792-799).

The antibody that binds PTK7 may prevent CCN ligand binding. For example, the anti-PTK7 antibody may bind the ligand binding portion or ligand binding domain of PTK7. An antibody that prevents ligand binding may be monovalent or bivalent. By “monovalent” it is meant that the antibody only has a single binding site for PTK7. For example, Fab, Fv, ScFv and sdAb fragments are monovalent. By “bivalent” it is meant that the antibody has two binding sites for PTK7. For example, a whole antibody and F(ab′) 2 fragments are bivalent. In some embodiments, the antibody is a Fab fragment, an Fv fragment, an ScFv fragment, a F (ab)′2 fragment, or a whole antibody.

The antibody may bind to an epitope in the seven immunoglobulin-type loops of PTK7 (the extracellular portion of PTK7). In some embodiments, the anti-PTK7 antibody binds to one or more of the seven immunoglobulin-type loops of PTK7.

PTK7 belongs to the RTK family of receptors which are typically activated by receptor dimerization. An antibody of the invention may inhibit receptor dimerization. In some embodiments, an antibody that inhibits CCN ligand binding also inhibits receptor dimerization. For example, a monovalent antibody may be beneficial for preventing ligand binding and/or inhibiting receptor dimerization. The antibody described herein may inhibit a GPC (e.g., GPC1, GPC2, GPC3, GPC4, GPC5, or GPC6) from binding PTK7. Antibodies that inhibit CCN ligand binding to PTK7 may also inhibit coreceptors from associating with the receptor (i.e., by steric hinderance).

In some embodiments, the antibody may promote PTK7 internalisation. In this case, the antibody will remove PTK7 from the cell surface. Again, the antibody may be monovalent or bivalent. For example, a bivalent antibody may be beneficial in promoting PTK7 internalisation. A bivalent antibody may induce receptor internalisation by causing homodimerization of PTK7. In some cases, the antibody inhibits ligand binding and promotes internalisation of PTK7. Promoting internalisation of PTK7 beneficially reduces the levels of PTK7 on the cell surface. The internalised PTK7 may undergo proteasomal degradation or internalised PTK7 may induce apoptosis.

The antibody may also bind to PTK7 and prevent PTK7 associating with/binding to a GPC. The antibody may bind to PTK7 and block the portion of PTK7 that interacts with a GPC. PTK7 may associate with a GPC directly or indirectly via a CCN ligand.

Anti-PTK7 antibodies which may find use in the present invention include, but are not limited to, Cofetuzumab (unconjugated) also called “hSC6.24” (SEQ ID NOs: 64 & 65 in U.S. Pat. No. 10,836,831), “hSC6.23” (SEQ ID NOs; 62 &63 in U.S. Pat. No. 10,836,831). “hSC6.41” (SEQ ID NOs; 66 & 67 in U.S. Pat. No. 10,836,831), “hSC6.58” (SEQ ID NOs: 68 & 69 in U.S. Pat. No. 10,836,831), “SC6.2.35” (SEQ ID NOs: 20 & 21 in U.S. Pat. No. 10,836,831), “SC6.10.2” (SEQ ID NOs: 22 & 23 in U.S. Pat. No. 10,836,831), “SC6.21” (SEQ ID NOs: 46 & 47 in U.S. Pat. No. 10,836,831), “7C8” (SEQ ID NOs: 4 & 10 in US20120027782), “12C6” (SEQ ID NOs: 3 & 8 in US20120027782), and “12C6a” (SEQ ID NOs: 3 & 9 in US20120027782) “3G8” (SEQ ID NOs: 1 & 5 in US20120027782), “3G8a” (SEQ ID NOs: 1 & 6 in US20120027782), “4D5” (SEQ ID NOs: 2 & 7 in US20120027782).

In some embodiments, the agent that binds to a GPC and inhibits GPC-CCN ligand-PTK7 complex formation is an antibody. In some embodiments, the GPC is selected from the group consisting of GPC1, GPC2, GPC3, GPC4, GPC5, and GPC6. In preferred embodiments, the GPC is GPC1 or GPC4. As with the anti-PTK7 antibody, the anti-GPC antibody may be monovalent or bivalent. In some embodiments, the antibody is a Fab fragment, an Fv fragment, an ScFv fragment, a F (ab)′2 fragment, or a whole antibody.

It is anticipated that upon ligand binding, the coreceptor (i.e., GPC) binds to/associates with PTK7, thus forming a GPC-CCN ligand-PTK7 complex. In some embodiments, the antibody binds to a GPC (i.e., GPC1, GPC2, GPC3, GPC4, GPC5, and/or GPC6) and inhibits CCN ligand (e.g., CCN protein or fragment thereof) from binding. The antibody may inhibit the formation of a GPC-CCN ligand-PTK7 complex by inhibiting CCN ligand binding to a GPC in the first place.

In preferred embodiments, the antibody binds to GPC1 or GPC4 and inhibits CCN ligand binding. The antibody may bind the ligand-binding portion of a GPC. In some embodiments, the antibody binds an epitope in the N-terminal portion of a GPC. In other embodiments, the antibody binds an epitope in the C-terminal portion of a GPC. In some embodiments, the epitope is free of heparan sulphate chains. In some embodiments, the antibody binds to a conformational epitope comprising a portion the N-terminal domain and/or a portion of the C-terminal domain.

In some embodiments, the antibody binds to a GPC (i.e., GPC1, GPC2, GPC3, GPC4, GPC5, and/or GPC6) and inhibits said GPC from binding to/associating with PTK7. In some embodiments, the antibody binds to a GPC (i.e., GPC1, GPC2, GPC3, GPC4, GPC5, and/or GPC6) comprising a CCN ligand and inhibits said GPC comprising a CCN ligand from binding to/associating with PTK7. In this case, the antibody inhibits the formation of a GPC-CCN ligand-PTK7 complex by inhibiting CCN ligand bound GPC from binding to/associating with PTK7.

Anti-GPC1 antibodies and single domain antibodies which may find use in the present invention include, but are not limited to, “AM4” from U.S. Pat. No. 10,577,418 and “HM2” and “D4” (mouse monoclonal antibody and single domain camelid, respectively) from WO2020/154150.

Anti-GPC2 antibodies which may find use in the present invention include, but are not limited to, “LH1”, “LH4” and “LH7” from U.S. Pat. No. 11,066,479.

Anti-GPC3 antibodies which may find use in the present invention include, but are not limited to, “Hd1.8Ld1.6”, “pH7 pL14”, “pH7 pL16” and “HOLO” from WO2009/041062.

Anti-GPC4 single domain antibodies which may find use in the present invention include, but are not limited to, “RB1”, “RB3” and “RB3v” from WO2022/079270.

Monoclonal antibodies (mAbs) are useful in the methods of the invention and are a homogenous population of antibodies specifically targeting a single epitope on an antigen. Polyclonal antibodies are useful in the methods of the invention. Monospecific polyclonal antibodies are preferred. Suitable polyclonal and monoclonal antibodies can be prepared using methods well known in the art.

Antigen binding fragments of antibodies, such as Fab and Fab2 fragments may also be used/provided as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” is meant molecules wherein the VH and VL partner domains are covalently linked, e.g., by a flexible oligopeptide.

Fab, Fv, ScFv and dAb antibody fragments can all be expressed in and secreted from E. coli, thus allowing the facile production of large amounts of the said fragments. Synthetic antibodies which bind to PTK7, GPC1, GPC2, GPC3, GPC4, GPC5, or GPC6 may also be made using phage display technology as is well known in the art.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol.154 (7): 331 0-15 9 (1995); and Hawkins et al, J. Mol. Biol. 226:889-896 (1992).

Antibodies according to the present invention may exhibit specific binding to PTK7, GPC1, GPC2, GPC3, GPC4, GPC5, or GPC6, preferably PTK7, GPC1, or GPC4. An agent, in this case an antibody, that specifically binds to a target molecule may bind the target with greater affinity, and/or with greater duration than it binds to other targets. In one embodiment, the extent of binding of an antibody to an unrelated target is less than about 10% of the binding of the antibody to the target as measured, e.g., by ELISA, or by a radioimmunoassay (RIA). Alternatively, the binding specificity may be reflected in terms of binding affinity where the antibody binds to PTK7, GPC1, GPC2, GPC3, GPC4, GPC5, or GPC6 with a KD that is at least 0.1 order of magnitude (i.e., 0.1×10n, where n is an integer representing the order of magnitude) greater than the KD of the antibody towards another target molecule. This may optionally be one of at least 0.2, 0.3, 0.4, 0.5, 0.6, 0.7, 0.8, 0.9, 1.0, 1.5, or 2.0.

In some embodiments, the anti-GPC antibody binds to more than one GPC family member. For example, an antibody that binds to GPC4 may also bind to GPC6 since they share around 63% homology. However, in preferred embodiments, the anti-GPC antibody binds to a single GPC family member only (i.e., it is not cross-reactive with other GPC family members).

Antibodies may be provided in isolated or purified form. The antibodies described herein may be formulated as a pharmaceutical composition or medicament.

In other embodiments, the agent that binds to PTK7 and has PTK7 antagonist activity is an antibody-like molecule. In some embodiments, the agent that binds to a GPC and inhibits GPC-CCN ligand-PTK7 complex formation is an antibody-like molecule. In some embodiments, the antibody-like molecule is an adnectin, an affibody, an affilin, an affimer, an affitin, an alphabody, an anticalin, an avimer, a DARPin, a fynomer, a knottin, a monobody, or a nanobody. An antibody-like molecule binds to its target similarly to the specific binding of an antibody.

### Aptamers

In some embodiments, the agent that binds to PTK7 and has PTK7 antagonist activity is an aptamer.

The aptamer may bind to one of the seven immunoglobulin-type loops of PTK7 (the extracellular portion of PTK7). In some embodiments, the aptamer binds to one or more of the seven immunoglobulin-type loops of PTK7.

The aptamer may inhibit ligand binding to PTK7. The aptamer may be monovalent or multivalent. In some embodiments, an aptamer binding to PTK7 prevents PTK7 from associating with a GPC. The aptamer may bind to PTK7 and block the portion of PTK7 that interacts with a GPC. PTK7 may associate with a GPC directly or indirectly via a CCN ligand.

An aptamer of the invention may inhibit PTK7 dimerization. In some embodiments, the aptamer that prevents ligand binding also prevents PTK7 dimerization. For example, a monovalent aptamer may be beneficial for preventing ligand binding and/or inhibiting receptor dimerization. Aptamers that inhibit ligand binding to PTK7 may also prevent coreceptors from associating with the receptor (i.e., by steric hinderance).

In some embodiments, the aptamer may promote PTK7 internalisation, for example, “sgc8”, a DNA aptamer (Xiao Z et al., Chem. Eur. J. 2008, 14, 1769-1775). In this case, the aptamer will remove PTK7 from the cell surface. For example, a multivalent or bivalent aptamer may be beneficial in PTK7 internalisation. A multivalent aptamer may induce receptor internalisation by causing homodimerization of PTK7. In some cases, the aptamer inhibits ligand binding and promotes internalisation of PTK7. In some embodiments, the aptamer is sgc8, or a derivative thereof (for example, sgc8c).

In other embodiments, the agent that binds to a GPC (i.e., GPC1, GPC2, GPC3, GPC4, GPC5, GPC6) and inhibits GPC-CCN ligand-PTK7 complex formation is an aptamer. The aptamer may bind to a GPC and inhibit CCN ligand binding to said GPC. The aptamer may bind to a GPC and inhibit said GPC from associating with PTK7, optionally said aptamer may bind to a GPC comprising a CCN ligand (i.e., CCN ligand bound GPC) and inhibit said GPC comprising a CCN ligand from binding to/associating with PTK7. In some embodiments, the aptamer inhibits PTK7 signalling.

As with the aptamer for PTK7, the aptamer targeting a GPC may be monovalent or bivalent.

Aptamers, also called nucleic acid ligands, are nucleic acid molecules characterised by the ability to bind to a target molecule with high specificity and high affinity. Almost every aptamer identified to date is a non-naturally occurring molecule. The aptamer may be a DNA aptamer or an RNA aptamer.

Aptamers to a given target (e.g., PTK7, GPC1, GPC2, GPC3, GPC4, GPC5, or GPC6) may be identified and/or produced by the method of Systematic Evolution of Ligands by Exponential enrichment (SELEX™). Aptamers and SELEX are described in Tuerk and Gold (Systematic evolution of ligands by exponential enrichment: RNA ligands to bacteriophage T4 DNA polymerase. Science. 1990 Aug. 3; 249 (4968): 505-10) and in WO91/19813.

Aptamers may be DNA or RNA molecules and may be single stranded or double stranded. The aptamer may comprise chemically modified nucleic acids, for example, in which the sugar and/or phosphate and/or base is chemically modified. Such modifications may improve the stability of the aptamer or make the aptamer more resistant to degradation and may include modification at the 2′ position of ribose. Aptamers may be synthesised by methods which are well known to the skilled person. For example, aptamers may be chemically synthesised, e.g., on a solid support.

Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer.

Aptamers can be thought of as the nucleic acid equivalent of monoclonal antibodies and often have Kd's in the nM or pM range, e.g., less than one of 500 nM, 100 nM, 50 nM, 10 nM, 1 nM, 500 pM, 100 pM. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule.

Aptamers according to the present invention may be provided in purified or isolated form. Aptamers according to the present invention may be formulated as a pharmaceutical composition or medicament. Suitable aptamers may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. Suitable aptamers may optionally have a maximum length of one of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. Suitable aptamers may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

### Bifunctional Molecules

In some embodiments, the agent is a bifunctional molecule. The bifunctional molecule may promote the internalisation and degradation of PTK7 or a GPC. For example, the agent may be a lysosome-targeting chimera (LYTAC).

A LYTAC is a bifunctional molecule that is capable of binding both a cell-surface lysosome shuttling receptor and a membrane-bound target protein or an extracellular domain of said target protein (Banik S M et al., 2020, Nature, 584 (7820): 291-297, Ahn G et al., 2021, Nat Chem Biol 17 (9): 937-946 and WO2020132100). Example cell-surface lysosome shuttling receptors comprise the cation-independent mannose-6-phsophate receptor (CI-M6PR, also known as the insulin-like growth factor 2 (IGF-II) receptor) or the asialoglycoprotein receptor (ASGPR). Other example cell-surface lysosome shuttling receptors include sortilin folate receptor, IFITM3, LIMP1, and LIMP2. A bifunctional molecule as disclosed herein may comprise a target binding moiety and a cell-surface lysosome shuttling receptor binding moiety. In some embodiments, the bifunctional molecule further comprises a linker between the target binding moiety and the cell-surface lysosome shuttling receptor.

In some embodiments, the cell-surface lysosome shuttling receptor binding moiety is a CI-M6PR binding moiety. Suitable CI-M6PR binding moieties include an antibody, an antibody-like molecule, an aptamer and a CI-M6PR ligand. CI-M6PR ligands include one or more mannose-6-phophates (M6P or M6Pn). Alternatively, the CI-M6PR ligand may be one or more M6P analogs (such as phosphonate M6P, malonate M6P, etc.). In some embodiments, the CI-M6PR binding moiety is a glycoprotein (e.g., N-carboxyanhydride (NCA)-derived glycoprotein) comprising one or more amino acids functionalised with one or more CI-M6PR ligands (as described in WO2020132100). In some embodiments, the CI-M6PR binding moiety is an aptamer having the following sequence (SEQ ID NO: 31; Miao et al., Angew. Chem. Int. Ed. 2021, 60, 11267-11271):

5′-GGGCGCGTAGATGACGAGCAGTCCTAACATCGTTTAGGAC-3′

In another embodiment, the cell-surface lysosome shuttling receptor binding moiety is an ASGPR binding moiety. Suitable ASGPR binding moieties include an antibody, an antibody-like molecule, an aptamer, or an ASGPR ligand. In some embodiments, the ASGPR ligand is a moiety comprising N-acetylgalactosamines (GalNAc), galactose or glucose. In some embodiments, the ASGPR ligand is a polymer scaffold or a dendrimer comprising one or more of GalNac, galactose, or glucose.

Thus, in the context of the present invention, the agent that binds to PTK7 and has PTK7 antagonist activity may be a bifunctional molecule comprising a PTK7 binding moiety and a cell-surface lysosome shuttling receptor binding moiety. In some embodiments, the PTK7 binding moiety is an antibody, an antibody-like molecule, an aptamer, or a CCN-ligand. In some embodiments, the PTK7 binding antibody is selected from any one of the anti-PTK7 antibodies as disclosed herein. In some embodiments, the PTK7 binding aptamer is sgc8, or a derivative thereof (for example, C8FL and other derivatives described in Shangguan D et al., Chembiochem. 2007 Apr. 16; 8 (6): 603-6). In some embodiments, the aptamer has the following sequence (SEQ ID NO: 32; sgc8c as described in Miao et al., Angew. Chem. Int. Ed. 2021, 60, 11267-11271):

5′-ATCTAACTGCTGCGCCGCCGGGAAAATACTGTACGGTTAGA-3′

Also anticipated are bifunctional molecules that bind to a GPC and inhibit GPC-CCN ligand-PTK7 complex formation. The bifunctional molecules comprise a GPC binding moiety and a cell-surface lysosome shuttling receptor binding moiety. In some embodiments, the GPC binding moiety is an antibody, an antibody-like molecule, an aptamer, or a CCN-ligand. In some embodiments, the GPC is GPC1, GPC2, GPC3, GPC4, GPC5, or GPC6. In some embodiments, the GPC is GPC1 or GPC4.

Another example of a bifunctional molecule is an antibody, or an antibody-like molecule functionalized to promote lysosome targeting in addition to PTK7 or GPC targeting, e.g., in the form of a bispecific antibody which comprises a cell-surface lysosome shuttling receptor (e.g., CI-MGPR) binding portion and a PTK7 or GPC binding portion.

### Agents Capable of Reducing Expression of PTK7, GPC1, GPC2, GPC3, GPC4, GPC5, or GPC6

The present invention provides an agent that inhibits expression of PTK7 for use in a method of treatment or prophylaxis of disease. In some embodiments, the agent that inhibits expression of PTK7 is selected from the group consisting of an agent that inhibits transcription of the gene encoding PTK7, an agent that inhibits post-transcriptional processing of RNA encoding PTK7, an agent that destabilises RNA encoding PTK7, or an agent that promotes degradation of RNA encoding PTK7.

Another aspect of the invention provides an agent that inhibits expression of a GPC. In some embodiments, the agent inhibits expression of GPC1, GPC2, GPC3, GPC4, GPC5, or GPC6. In preferred embodiments, the agent inhibits expression of GPC1 or GPC4. In some embodiments, the agent that inhibits expression of a GPC is selected from the group consisting of an agent that inhibits transcription of the gene encoding a GPC, an agent that inhibits post-transcriptional processing of RNA encoding a GPC, an agent that destabilises RNA encoding a GPC, or an agent that promotes degradation of RNA encoding a GPC.

“Inhibits expression” includes reduction of PTK7 or a GPC to any degree. In other words, expression may be around 5%, 10%, 15%, 20%, 25%, 30%, 35%, 40%, 45%, 50%, 55%, 60%, 65%, 70%, 75%, 80%, 85%, 90% or 95% of the level found in untreated cells. In the context of the invention, the term “expression” may encompass total cell expression or plasma membrane levels of PTK7 or GPC.

The agents described herein are anticipated to inhibit CCN ligand-induced signalling. As described herein, CCN ligand binding to PTK7 (i.e., CCN ligand-induced signalling) is anticipated to result in the phosphorylation of Akt and/or phosphorylation of ERK1/2.

Expression may be gene or protein expression and may be determined as described herein or by methods in the art that will be well known to a skilled person. Expression may be by a cell/tissue/organ/organ system of a subject.

Suitable agents may be of any kind, but in some embodiments, an agent capable of inhibiting or reducing the expression of PTK7 or any one of GPC1, GPC2, GPC3, GPC4, GPC5, or GPC6 may be a small molecule or an oligonucleotide.

In addition to the above, an agent capable of inhibiting or reducing the expression of PTK7 or any one of GPC1, GPC2, GPC3, GPC4, GPC5, or GPC6 may do so e.g., through inhibiting post-translation processing of PTK7, GPC1, GPC2, GPC3, GPC4, GPC5, or GPC6 polypeptide, destabilising PTK7, GPC1, GPC2, GPC3, GPC4, GPC5, or GPC6 polypeptide or promoting degradation of PTK7, GPC1, GPC2, GPC3, GPC4, GPC5, or GPC6 polypeptide.

In some embodiments, the agent is an ASO or an oligonucleotide which causes RNA interference (RNAi).

The present invention contemplates the use of antisense nucleic acid to prevent/reduce expression of PTK7, GPC1, GPC2, GPC3, GPC4, GPC5, or GPC6. In some embodiments, an agent capable of inhibiting or reducing the expression of PTK7, GPC1, GPC2, GPC3, GPC4, GPC5, or GPC may cause reduced expression by RNA interference (RNAi).

In some embodiments, the agent may be an inhibitory nucleic acid, such as antisense or small interfering RNA, including but not limited to shRNA or siRNA. In some embodiments, the inhibitory nucleic acid is provided in a vector. For example, in some embodiments the agent may be a lentiviral vector encoding shRNA for one or more of PTK7, GPC1, GPC2, GPC3, GPC4, GPC5, or GPC6.

Oligonucleotide molecules, particularly RNA, may be employed to regulate gene expression. These include antisense oligonucleotides, targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

An antisense oligonucleotide (ASO) is an oligonucleotide, preferably single stranded that targets and binds, by complementary sequence binding, to a target oligonucleotide, e.g., mRNA. Where the target oligonucleotide is an mRNA, binding of the antisense to the mRNA blocks translation of the mRNA and expression of the gene product. Antisense oligonucleotides may be designed to bind sense genomic nucleic acid and inhibit transcription or promote degradation of a target nucleotide sequence.

In view of the known nucleic acid sequences for PTK7, GPC1, GPC2, GPC3, GPC4, GPC5, or GPC6 (exemplary human mRNA sequence variants are available from GenBank® under accession numbers NM_002821.5 (PTK7, SEQ ID NO 9), NM_002081.3 (GPC1, SEQ ID NO 10), NM_152742.3 (GPC2, SEQ ID NO 11), NM_001164617.2 (GPC3 isoform P51654-3, SEQ ID NO 12), NM_004484.4 (GPC3 isoform P51654-1, SEQ ID NO 14), NM_001448.3 (GPC4, SEQ ID NO 15), NM_004466.6 (GPC5, SEQ ID NO 16), NM_005708.5 (GPC6, SEQ ID NO 17, see Table 1), oligonucleotides may be designed to repress or silence the expression of PTK7, GPC1, GPC2, GPC3, GPC4, GPC5, or GPC6. Such oligonucleotides may have any length, but may preferably be short, e.g., less than 100 nucleotides, e.g., 10-40 nucleotides, or 20-50 nucleotides, and may comprise a nucleotide sequence having complete- or near-complementarity (e.g., 80%, 85%, 90%, 91%, 92%, 93%, 94%, 95%, 96%, 97%, 98%, 99% or 100% complementarity) to a sequence of nucleotides of corresponding length in the target oligonucleotide, e.g., PTK7, GPC1, GPC2, GPC3, GPC4, GPC5, or GPC6. The complementary region of the nucleotide sequence may have any length, but is preferably at least 5, and optionally no more than 50, nucleotides long, e.g. one of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. Where the target is GPC3, the oligonucleotide may preferably target the P51654-3 isoform.

TABLE 1
Exemplary human mRNA sequence variants

| GenBank | |
|---|---|
| accession no. | Sequence |
| PTK7: | SEQ ID NO: 9 |
| NM_002821.5 | ACTGGGCGCGCGCGGCTCCGGCTCGGGACGCCTCGGGACGCCTCGGGGTCGGGCTCCGGC |
| | TGCGGCTGCTGCTGCGGCGCCCGCGCTCCGGTGCGCTCCGCCTCCTGTGCCCGCCGCGGA |
| | GCGCAGTCTGCGCGCCCGCCGTGCGCCCTCAGCTCCTTTTCCTGAGCCCGCCGCGATGGG |
| | AGCTGCGCGGGGATCCCCGGCCAGACCCCGCCGGTTGCCTCTGCTCAGCGTCCTGCTGCT |
| | GCCGCTGCTGGGCGGTACCCAGACAGCCATTGTCTTCATCAAGCAGCCGTCCTCCCAGGA |
| | TGCACTGCAGGGGCGCCGGGCGCTGCTTCGCTGTGAGGTTGAGGCTCCGGGCCCGGTACA |
| | TGTGTACTGGCTGCTCGATGGGGCCCCTGTCCAGGACACGGAGCGGCGTTTCGCCCAGGG |
| | CAGCAGCCTGAGCTTTGCAGCTGTGGACCGGCTGCAGGACTCTGGCACCTTCCAGTGTGT |
| | GGCTCGGGATGATGTCACTGGAGAAGAAGCCCGCAGTGCCAACGCCTCCTTCAACATCAA |
| | ATGGATTGAGGCAGGTCCTGTGGTCCTGAAGCATCCAGCCTCGGAAGCTGAGATCCAGCC |
| | ACAGACCCAGGTCACACTTCGTTGCCACATTGATGGGCACCCTCGGCCCACCTACCAATG |
| | GTTCCGAGATGGGACCCCCCTTTCTGATGGTCAGAGCAACCACACAGTCAGCAGCAAGGA |
| | GCGGAACCTGACGCTCCGGCCAGCTGGTCCTGAGCATAGTGGGCTGTATTCCTGCTGCGC |
| | CCACAGTGCTTTTGGCCAGGCTTGCAGCAGCCAGAACTTCACCTTGAGCATTGCTGATGA |
| | AAGCTTTGCCAGGGTGGTGCTGGCACCCCAGGACGTGGTAGTAGCGAGGTATGAGGAGGC |
| | CATGTTCCATTGCCAGTTCTCAGCCCAGCCACCCCCGAGCCTGCAGTGGCTCTTTGAGGA |
| | TGAGACTCCCATCACTAACCGCAGTCGCCCCCCACACCTCCGCAGAGCCACAGTGTTTGC |
| | CAACGGGTCTCTGCTGCTGACCCAGGTCCGGCCACGCAATGCAGGGATCTACCGCTGCAT |
| | TGGCCAGGGGCAGAGGGGCCCACCCATCATCCTGGAAGCCACACTTCACCTAGCAGAGAT |
| | TGAAGACATGCCGCTATTTGAGCCACGGGTGTTTACAGCTGGCAGCGAGGAGCGTGTGAC |
| | CTGCCTTCCCCCCAAGGGTCTGCCAGAGCCCAGCGTGTGGTGGGAGCACGCGGGAGTCCG |
| | GCTGCCCACCCATGGCAGGGTCTACCAGAAGGGCCACGAGCTGGTGTTGGCCAATATTGC |
| | TGAAAGTGATGCTGGTGTCTACACCTGCCACGCGGCCAACCTGGCTGGTCAGCGGAGACA |
| | GGATGTCAACATCACTGTGGCCACTGTGCCCTCCTGGCTGAAGAAGCCCCAAGACAGCCA |
| | GCTGGAGGAGGGCAAACCCGGCTACTTGGATTGCCTGACCCAGGCCACACCAAAACCTAC |
| | AGTTGTCTGGTACAGAAACCAGATGCTCATCTCAGAGGACTCACGGTTCGAGGTCTTCAA |
| | GAATGGGACCTTGCGCATCAACAGCGTGGAGGTGTATGATGGGACATGGTACCGTTGTAT |
| | GAGCAGCACCCCAGCCGGCAGCATCGAGGCGCAAGCCCGTGTCCAAGTGCTGGAAAAGCT |
| | CAAGTTCACACCACCACCCCAGCCACAGCAGTGCATGGAGTTTGACAAGGAGGCCACGGT |
| | GCCCTGTTCAGCCACAGGCCGAGAGAAGCCCACTATTAAGTGGGAACGGGCAGATGGGAG |
| | CAGCCTCCCAGAGTGGGTGACAGACAACGCTGGGACCCTGCATTTTGCCCGGGTGACTCG |
| | AGATGACGCTGGCAACTACACTTGCATTGCCTCCAACGGGCCGCAGGGCCAGATTCGTGC |
| | CCATGTCCAGCTCACTGTGGCAGTTTTTATCACCTTCAAAGTGGAACCAGAGCGTACGAC |
| | TGTGTACCAGGGCCACACAGCCCTACTGCAGTGCGAGGCCCAGGGGGACCCCAAGCCGCT |
| | GATTCAGTGGAAAGGCAAGGACCGCATCCTGGACCCCACCAAGCTGGGACCCAGGATGCA |
| | CATCTTCCAGAATGGCTCCCTGGTGATCCATGACGTGGCCCCTGAGGACTCAGGCCGCTA |
| | CACCTGCATTGCAGGCAACAGCTGCAACATCAAGCACACGGAGGCCCCCCTCTATGTCGT |
| | GGACAAGCCTGTGCCGGAGGAGTCGGAGGGCCCTGGCAGCCCTCCCCCCTACAAGATGAT |
| | CCAGACCATTGGGTTGTCGGTGGGTGCCGCTGTGGCCTACATCATTGCCGTGCTGGGCCT |
| | CATGTTCTACTGCAAGAAGCGCTGCAAAGCCAAGCGGCTGCAGAAGCAGCCCGAGGGCGA |
| | GGAGCCAGAGATGGAATGCCTCAACGGTGGGCCTTTGCAGAACGGGCAGCCCTCAGCAGA |
| | GATCCAAGAAGAAGTGGCCTTGACCAGCTTGGGCTCCGGCCCCGCGGCCACCAACAAACG |
| | CCACAGCACAAGTGATAAGATGCACTTCCCACGGTCTAGCCTGCAGCCCATCACCACGCT |
| | GGGGAAGAGTGAGTTTGGGGAGGTGTTCCTGGCAAAGGCTCAGGGCTTGGAGGAGGGAGT |
| | GGCAGAGACCCTGGTACTTGTGAAGAGCCTGCAGAGCAAGGATGAGCAGCAGCAGCTGGA |
| | CTTCCGGAGGGAGTTGGAGATGTTTGGGAAGCTGAACCACGCCAACGTGGTGCGGCTCCT |
| | GGGGCTGTGCCGGGAGGCTGAGCCCCACTACATGGTGCTGGAATATGTGGATCTGGGAGA |
| | CCTCAAGCAGTTCCTGAGGATTTCCAAGAGCAAGGATGAAAAATTGAAGTCACAGCCCCT |
| | CAGCACCAAGCAGAAGGTGGCCCTATGCACCCAGGTAGCCCTGGGCATGGAGCACCTGTC |
| | CAACAACCGCTTTGTGCATAAGGACTTGGCTGCGCGTAACTGCCTGGTCAGTGCCCAGAG |
| | ACAAGTGAAGGTGTCTGCCCTGGGCCTCAGCAAGGATGTGTACAACAGTGAGTACTACCA |
| | CTTCCGCCAGGCCTGGGTGCCGCTGCGCTGGATGTCCCCCGAGGCCATCCTGGAGGGTGA |
| | CTTCTCTACCAAGTCTGATGTCTGGGCCTTCGGTGTGCTGATGTGGGAAGTGTTTACACA |
| | TGGAGAGATGCCCCATGGTGGGCAGGCAGATGATGAAGTACTGGCAGATTTGCAGGCTGG |
| | GAAGGCTAGACTTCCTCAGCCCGAGGGCTGCCCTTCCAAACTCTATCGGCTGATGCAGCG |
| | CTGCTGGGCCCTCAGCCCCAAGGACCGGCCCTCCTTCAGTGAGATTGCCAGCGCCCTGGG |
| | AGACAGCACCGTGGACAGCAAGCCGTGAGGAGGGAGCCCGCTCAGGATGGCCTGGGCAGG |
| | GGAGGACATCTCTAGAGGGAAGCTCACAGCATGATGGGCAAGATCCCTGTCCTCCTGGGC |
| | CCTGAGGCCCCTGCCCTAGTGCAACAGGCATTGCTGAGGTCTGAGCAGGGCCTGGCCTTT |
| | CCTCCTCTTCCTCACCCTCATCCTTTGGGAGGCTGACTTGGACCCAAACTGGGCGACTAG |
| | GGCTTTGAGCTGGGCAGTTTTCCCTGCCACCTCTTCCTCTATCAGGGACAGTGTGGGTGC |
| | CACAGGTAACCCCAATTTCTGGCCTTCAACTTCTCCCCTTGACCGGGTCCAACTCTGCCA |
| | CTCATCTGCCAACTTTGCCTGGGGAGGGCTAGGCTTGGGATGAGCTGGGTTTGTGGGGAG |
| | TTCCTTAATATTCTCAAGTTCTGGGCACACAGGGTTAATGAGTCTCTTGGCCCACTGGTC |
| | CCACTTGGGGGTCTAGACCAGGATTATAGAGGACACAGCAAGTGAGTCCTCCCCACTCTG |
| | GGCTTGTGCACACTGACCCAGACCCACGTCTTCCCCACCCTTCTCTCCTTTCCTCATCCT |
| | AAGTGCCTGGCAGATGAAGGAGTTTTCAGGAGCTTTTGACACTATATAAACCGCCCTTTT |
| | TGTATGCACCACGGGCGGCTTTTATATGTAATTGCAGCGTGGGGTGGGTGGGCATGGGAG |
| | GTAGGGGTGGGCCCTGGAGATGAGGAGGGTGGGCCATCCTTACCCCACACTTTTATTGTT |
| | GTCGTTTTTTGTTTGTTTTGTTTTTTTGTTTTTGTTTTTGTTTTTACACTCGCTGCTCTC |
| | AATAAATAAGCCTTTTTTACAA |
| GPC1: | SEQ ID NO: 10 |
| NM_002081.3 | GCCGAGCCGGGACTGCGCTAGCCCGCCGCGCTCTGGGCTGCCCGAGCGAGCGTTCGGACC |
| | TCGCACCCCGCGCGCCCCGCGCCGCCGCCGCCGCCGGCTTTTGTTGTCTCCGCCTCCTCG |
| | GCCGCCGCCGCCTCTGGACCGCGAGCCGCGCGCGCCGGGACCTTGGCTCTGCCCTTCGCG |
| | GGCGGGAACTGCGCAGGACCCGGCCAGGATCCGAGAGAGGCGCGGGCGGGTGGCCGGGGG |
| | CGCCGCCGGCCCCGCCATGGAGCTCCGGGCCCGAGGCTGGTGGCTGCTATGTGCGGCCGC |
| | AGCGCTGGTCGCCTGCGCCCGCGGGGACCCGGCCAGCAAGAGCCGGAGCTGCGGCGAGGT |
| | CCGCCAGATCTACGGAGCCAAGGGCTTCAGCCTGAGCGACGTGCCCCAGGCGGAGATCTC |
| | GGGTGAGCACCTGCGGATCTGTCCCCAGGGCTACACCTGCTGCACCAGCGAGATGGAGGA |
| | GAACCTGGCCAACCGCAGCCATGCCGAGCTGGAGACCGCGCTCCGGGACAGCAGCCGCGT |
| | CCTGCAGGCCATGCTTGCCACCCAGCTGCGCAGCTTCGATGACCACTTCCAGCACCTGCT |
| | GAACGACTCGGAGCGGACGCTGCAGGCCACCTTCCCCGGCGCCTTCGGAGAGCTGTACAC |
| | GCAGAACGCGAGGGCCTTCCGGGACCTGTACTCAGAGCTGCGCCTGTACTACCGCGGTGC |
| | CAACCTGCACCTGGAGGAGACGCTGGCCGAGTTCTGGGCCCGCCTGCTCGAGCGCCTCTT |
| | CAAGCAGCTGCACCCCCAGCTGCTGCTGCCTGATGACTACCTGGACTGCCTGGGCAAGCA |
| | GGCCGAGGCGCTGCGGCCCTTCGGGGAGGCCCCGAGAGAGCTGCGCCTGCGGGCCACCCG |
| | TGCCTTCGTGGCTGCTCGCTCCTTTGTGCAGGGCCTGGGCGTGGCCAGCGACGTGGTCCG |
| | GAAAGTGGCTCAGGTCCCCCTGGGCCCGGAGTGCTCGAGAGCTGTCATGAAGCTGGTCTA |
| | CTGTGCTCACTGCCTGGGAGTCCCCGGCGCCAGGCCCTGCCCTGACTATTGCCGAAATGT |
| | GCTCAAGGGCTGCCTTGCCAACCAGGCCGACCTGGACGCCGAGTGGAGGAACCTCCTGGA |
| | CTCCATGGTGCTCATCACCGACAAGTTCTGGGGTACATCGGGTGTGGAGAGTGTCATCGG |
| | CAGCGTGCACACGTGGCTGGCGGAGGCCATCAACGCCCTCCAGGACAACAGGGACACGCT |
| | CACGGCCAAGGTCATCCAGGGCTGCGGGAACCCCAAGGTCAACCCCCAGGGCCCCGGGCC |
| | TGAGGAGAAGCGGCGCCGGGGCAAGCTGGCCCCGCGGGAGAGGCCACCTTCAGGCACGCT |
| | GGAGAAGCTGGTCTCCGAAGCCAAGGCCCAGCTCCGCGACGTCCAGGACTTCTGGATCAG |
| | CCTCCCAGGGACACTGTGCAGTGAGAAGATGGCCCTGAGCACTGCCAGTGATGACCGCTG |
| | CTGGAACGGGATGGCCAGAGGCCGGTACCTCCCCGAGGTCATGGGTGACGGCCTGGCCAA |
| | CCAGATCAACAACCCCGAGGTGGAGGTGGACATCACCAAGCCGGACATGACCATCCGGCA |
| | GCAGATCATGCAGCTGAAGATCATGACCAACCGGCTGCGCAGCGCCTACAACGGCAACGA |
| | CGTGGACTTCCAGGACGCCAGTGACGACGGCAGCGGCTCGGGCAGCGGTGATGGCTGTCT |
| | GGATGACCTCTGCAGCCGGAAGGTCAGCAGGAAGAGCTCCAGCTCCCGGACGCCCTTGAC |
| | CCATGCCCTCCCAGGCCTGTCAGAGCAGGAAGGACAGAAGACCTCGGCTGCCAGCTGCCC |
| | CCAGCCCCCGACCTTCCTCCTGCCCCTCCTCCTCTTCCTGGCCCTTACAGTAGCCAGGCC |
| | CCGGTGGCGGTAACTGCCCCAAGGCCCCAGGGACAGAGGCCAAGGACTGACTTTGCCAAA |
| | AATACAACACAGACGATATTTAATTCACCTCAGCCTGGAGAGGCCTGGGGTGGGACAGGG |
| | AGGGCCGGCGGCTCTGAGCAGGGGCAGGCGCAGAGGTCCCAGCCCCAGGCCTGGCCTCGC |
| | CTGCCTTTCTGCCTTTTAATTTTGTATGAGGTCCTCAGGTCAGCTGGGAGCCAGTGTGCC |
| | CAAAAGCCATGTATTTCAGGGACCTCAGGGGCACCTCCGGCTGCCTAGCCCTCCCCCCAG |
| | CTCCCTGCACCGCCGCAGAAGCAGCCCCTCGAGGCCTACAGAGGAGGCCTCAAAGCAACC |
| | CGCTGGAGCCCACAGCGAGCCTGTGCCTTCCTCCCCGCCTCCTCCCACTGGGACTCCCAG |
| | CAGAGCCCACCAGCCAGCCCTGGCCCACCCCCCAGCCTCCAGAGAAGCCCCGCACGGGCT |
| | GTCTGGGTGTCCGCCATCCAGGGTCTGGCAGAGCCTCTGAGATGATGCATGATGCCCTCC |
| | CCTCAGCGCAGGCTGCAGAGCCCGGCCCCACCTCCCTGCGCCCTTGAGGGGCCCCAGCGT |
| | CTGCAGGGTGACGCCTGAGACAGCACCACTGCTGAGGAGTCTGAGGACTGTCCTCCCACA |
| | GACCTGCAGTGAGGGGCCCTCCATGCGCAGATGAGGGGCCACTGACCCACCTGCGCTTCT |
| | GCTGGAGGAGGGGAAGCTGGGCCCAAAGGCCCAGGGAGGCAGCGTGGGCTCTGCCAATGT |
| | GGGCTGCCCCTCGCACACAGGGCTCACAGGGCAGGCCTTGCTGGGGTCCAGGGCTGTTGG |
| | AGGACCCCGAGGGCTGAGGAGCAGCCAGGACCCGCCTGCTCCCATCCTCACCCAGATCAG |
| | GAACCAGGGCCTCCCTGTTCACGGTGACACAGGTCAGGGCTCAGAGTGACCCTCAGCTGT |
| | CACCTGCTCACAGGGATGCTGGTGGCTGGTGAGACCCCGCACTGCAGACGGGAATGCCTA |
| | GGTCCCTTCCCGACCCAGCCAGCTGCAGGGCACGGGGACCTGGATAGTTAAGGGCTTTTC |
| | CAAACATGCATCCATTTACTGACACTTCCTGTCCTTGTTCATGGAGAGCTGTTCGCTCCT |
| | CCCAGATGGCTTCGGAGGGCCGCAGGGCCCACCTTGGACCCTGGTGACCTCCTGTCACTC |
| | ACTGAGGCCATCAGGGCCCTGCCCCAGGCCTGGACGGGCCCTCCTTCCCTCCTGTGCCCC |
| | AGCTGCCAGGCGGCCCTGGGGAGGGGTGGTGTGGTGTTGGGAAGGGGTCCTGCAGGGGGA |
| | GGAGGACTTGGAGGGTCTGGGGGCAGCTGTCCTGAACCGACTGACCCTGAGGAGGCCGCT |
| | TAGTGCTGCTTTGCTTTTCATCACCGTCCCGCACAGTGGACGGAGGTCCCCGGTTGCTGG |
| | TCAGGTCCCCATGGCTTGTTCTCTGGAACCTGACTTTAGATGTTTTGGGATCAGGAGCCC |
| | CCAACACAGGCAAGTCCACCCCATAATAACCCTGCCAGTGCCAGGGTGGGCTGGGGACTC |
| | TGGCACAGTGATGCCGGGCGCCAGGACAGCAGCACTCCCGCTGCACACAGACGGCCTAGG |
| | GGTGGCGCTCAGACCCCACCCTACGCTCATCTCTGGAAGGGGCAGCCCTGAGTGGTCACT |
| | GGTCAGGGCAGTGGCCAAGCCTGCTGTGTCCTTCCTCCACAAGGTCCCCCCACCGCTCAG |
| | TGTCAGCGGGTGACGTGTGTTCTTTTGAGTCCTTGTATGAATAAAAGGCTGGAAACCTA |
| GPC2: | SEQ ID NO: 11 |
| NM_152742.3 | GCTCCCATTGTCTCGGCAGATGCCGCCTGGTCCAGCTATCGTGCTCGGTATTCAGTTTTC |
| | CGGAGCAGCGCTCTTTCTCTGGCCCGCGGAGCGGTCCCGCGGCCGAGTACCGGATTCCCG |
| | AGTTTGGGAGGCTCTGCTTTCCTCCTTAGGACCCACTTTGCCGTCCTGGGGTGGCTGCAG |
| | TTATGTCCGCGCTGCGACCTCTCCTGCTTCTGCTGCTGCCTCTGTGTCCCGGTCCTGGTC |
| | CCGGACCCGGGAGCGAGGCAAAGGTCACCCGGAGTTGTGCAGAGACCCGGCAGGTGCTGG |
| | GGGCCCGGGGATATAGCTTAAACCTAATCCCTCCCGCCCTGATCTCAGGTGAGCACCTCC |
| | GGGTCTGTCCCCAGGAGTACACCTGCTGTTCCAGTGAGACAGAGCAGAGGCTGATCAGGG |
| | AGACTGAGGCCACCTTCCGAGGCCTGGTGGAGGACAGCGGCTCCTTTCTGGTTCACACAC |
| | TGGCTGCCAGGCACAGAAAATTTGATGAGTTTTTTCTGGAGATGCTCTCAGTAGCCCAGC |
| | ACTCTCTGACCCAGCTCTTCTCCCACTCCTACGGCCGCCTGTATGCCCAGCACGCCCTCA |
| | TATTCAATGGCCTGTTCTCTCGGCTGCGAGACTTCTATGGGGAATCTGGTGAGGGGTTGG |
| | ATGACACCCTGGCGGATTTCTGGGCACAGCTCCTGGAGAGAGTGTTCCCGCTGCTGCACC |
| | CACAGTACAGCTTCCCCCCTGACTACCTGCTCTGCCTCTCACGCTTGGCCTCATCTACCG |
| | ATGGCTCTCTGCAGCCCTTTGGGGACTCACCCCGCCGCCTCCGCCTGCAGATAACCCGGA |
| | CCCTGGTGGCTGCCCGAGCCTTTGTGCAGGGCCTGGAGACTGGAAGAAATGTGGTCAGCG |
| | AAGCGCTTAAGGTGCCGGTGTCTGAAGGCTGCAGCCAGGCTCTGATGCGTCTCATCGGCT |
| | GTCCCCTGTGCCGGGGGGTCCCCTCACTTATGCCCTGCCAGGGCTTCTGCCTCAACGTGG |
| | TTCGTGGCTGTCTCAGCAGCAGGGGACTGGAGCCTGACTGGGGCAACTATCTGGATGGTC |
| | TCCTGATCCTGGCTGATAAGCTCCAGGGCCCCTTTTCCTTTGAGCTGACGGCCGAGTCCA |
| | TTGGGGTGAAGATCTCGGAGGGTTTGATGTACCTGCAGGAAAACAGTGCGAAGGTGTCCG |
| | CCCAGGTGTTTCAGGAGTGCGGCCCCCCCGACCCGGTGCCTGCCCGCAACCGTCGAGCCC |
| | CGCCGCCCCGGGAAGAGGCGGGCCGGCTGTGGTCGATGGTGACCGAGGAGGAGCGGCCCA |
| | CGACGGCCGCAGGCACCAACCTGCACCGGCTGGTGTGGGAGCTCCGCGAGCGTCTGGCCC |
| | GGATGCGGGGCTTCTGGGCCCGGCTGTCCCTGACGGTGTGCGGAGACTCTCGCATGGCAG |
| | CGGACGCCTCGCTGGAGGCGGCGCCCTGCTGGACCGGAGCCGGGCGGGGCCGGTACTTGC |
| | CGCCAGTGGTCGGGGGCTCCCCGGCCGAGCAGGTCAACAACCCCGAGCTCAAGGTGGACG |
| | CCTCGGGCCCCGATGTCCCGACACGGCGGCGTCGGCTACAGCTCCGGGCGGCCACGGCCA |
| | GAATGAAAACGGCCGCACTGGGACACGACCTGGACGGGCAGGACGCGGATGAGGATGCCA |
| | GCGGCTCTGGAGGGGGACAGCAGTATGCAGATGACTGGATGGCTGGGGCTGTGGCTCCCC |
| | CAGCCCGGCCTCCTCGGCCTCCATACCCTCCTAGAAGGGATGGTTCTGGGGGCAAAGGAG |
| | GAGGTGGCAGTGCCCGCTACAACCAGGGCCGGAGCAGGAGTGGGGGGGCATCTATTGGTT |
| | TTCACACCCAAACCATCCTCATTCTCTCCCTCTCAGCCCTGGCCCTGCTTGGACCTCGAT |
| | AACGGGGGAGGGGTGCCCTAGCATCAGAAGGGTTCATGGCCCTTTCCCCTCCTCCCCCCT |
| | CAGCTGGGCCTGGGGAGGAGTCGAAGGGGGCTGCAGAGAGGGTAGAGAAGGGACTTTGCA |
| | GGTGAATGGCTGGGGCCCCAAATCCAGGAGATTTTCATCAGAGGTGGGTGGGTGTTCACA |
| | ATATTTATTTTTTCATTTGGTAATGGGAGGGGGGCCTGGGGGTATTTATTTAGGAGGGAG |
| | TGTGGTTTCCTTAGAAGGTATAGTCTCTAGCCCTCTAAGGCTGGGGCTGGTGATCAGCCC |
| | CAACAGAGAAAATGAGGAGTTTAGAGTTGCAGCTGGGGAAGGGGTTTGAAGGAAGTTGGA |
| | AGTGGGGAGGGGTGGGGGCATCTGGTCTCAGAAATGGACCAGCTGGATGCAGGGCAGGGG |
| | ACTGAGGGTGCTTGAGTAGGATGTGAGACTTCATGGGCCTGGGTTCTGTTGAGTTTTTTC |
| | AGTATCAATTTCTTAAACCAAATTTTAAAAAAAACAAGGTGGGGGGGTGCTCATCTCGTG |
| | ACCTCTGCCACCCACATCCTTCACAAACTCCATGTTTCAGTGTTTGAGTCCATGTTTATT |
| | CTGCAAATAAATGGTAATGTATTGGA |
| GPC3 (isoform | SEQ ID NO: 12 |
| P51654-3): | AGGTAGCTGCGAGGAAACTTTTGCAGCGGCTGGGTAGCAGCACGTCTCTTGCTCCTCAGG |
| NM_001164617.2 | GCCACTGCCAGGCTTGCCGAGTCCTGGGACTGCTCTCGCTCCGGCTGCCACTCTCCCGCG |
| | CTCTCCTAGCTCCCTGCGAAGCAGGATGGCCGGGACCGTGCGCACCGCGTGCTTGGTGGT |
| | GGCGATGCTGCTCAGCTTGGACTTCCCGGGACAGGCGCAGCCCCCGCCGCCGCCGCCGGA |
| | CGCCACCTGTCACCAAGTCCGCTCCTTCTTCCAGAGACTGCAGCCCGGACTCAAGTGGGT |
| | GCCAGAAACTCCCGTGCCAGGATCAGATTTGCAAGTATGTCTCCCTAAGGGCCCAACATG |
| | CTGCTCAAGAAAGATGGAAGAAAAATACCAACTAACAGCACGATTGAACATGGAACAGCT |
| | GCTTCAGTCTGCAAGTATGGAGCTCAAGTTCTTAATTATTCAGAATGCTGCGGTTTTCCA |
| | AGAGGCCTTTGAAATTGTTGTTCGCCATGCCAAGAACTACACCAATGCCATGTTCAAGAA |
| | CAACTACCCAAGCCTGACTCCACAAGCTTTTGAGTTTGTGGGTGAATTTTTCACAGATGT |
| | GTCTCTCTACATCTTGGGTTCTGACATCAATGTAGATGACATGGTCAATGAATTGTTTGA |
| | CAGCCTGTTTCCAGTCATCTATACCCAGCTAATGAACCCAGGCCTGCCTGATTCAGCCTT |
| | GGACATCAATGAGTGCCTCCGAGGAGCAAGACGTGACCTGAAAGTATTTGGGAATTTCCC |
| | CAAGCTTATTATGACCCAGGTTTCCAAGTCACTGCAAGTCACTAGGATCTTCCTTCAGGC |
| | TCTGAATCTTGGAATTGAAGTGATCAACACAACTGATCACCTGAAGTTCAGTAAGGACTG |
| | TGGCCGAATGCTCACCAGAATGTGGTACTGCTCTTACTGCCAGGGACTGATGATGGTTAA |
| | ACCCTGTGGCGGTTACTGCAATGTGGTCATGCAAGGCTGTATGGCAGGTGTGGTGGAGAT |
| | TGACAAGTACTGGAGAGAATACATTCTGTCCCTTGAAGAACTTGTGAATGGCATGTACAG |
| | AATCTATGACATGGAGAACGTACTGCTTGGTCTCTTTTCAACAATCCATGATTCTATCCA |
| | GTATGTCCAGAAGAATGCAGGAAAGCTGACCACCACTGAAACTGAGAAGAAAATATGGCA |
| | CTTCAAATATCCTATCTTCTTCCTGTGTATAGGGCTAGACTTACAGATTGGCAAGTTATG |
| | TGCCCATTCTCAACAACGCCAATATAGATCTGCTTATTATCCTGAAGATCTCTTTATTGA |
| | CAAGAAAGTATTAAAAGTTGCTCATGTAGAACATGAAGAAACCTTATCCAGCCGAAGAAG |
| | GGAACTAATTCAGAAGTTGAAGTCTTTCATCAGCTTCTATAGTGCTTTGCCTGGCTACAT |
| | CTGCAGCCATAGCCCTGTGGCGGAAAACGACACCCTTTGCTGGAATGGACAAGAACTCGT |
| | GGAGAGATACAGCCAAAAGGCAGCAAGGAATGGAATGAAAAACCAGTTCAATCTCCATGA |
| | GCTGAAAATGAAGGGCCCTGAGCCAGTGGTCAGTCAAATTATTGACAAACTGAAGCACAT |
| | TAACCAGCTCCTGAGAACCATGTCTATGCCCAAAGGTAGAGTTCTGGATAAAAACCTGGA |
| | TGAGGAAGGGTTTGAAAGTGGAGACTGCGGTGATGATGAAGATGAGTGCATTGGAGGCTC |
| | TGGTGATGGAATGATAAAAGTGAAGAATCAGCTCCGCTTCCTTGCAGAACTGGCCTATGA |
| | TCTGGATGTGGATGATGCGCCTGGAAACAGTCAGCAGGCAACTCCGAAGGACAACGAGAT |
| | AAGCACCTTTCACAACCTCGGGAACGTTCATTCCCCGCTGAAGCTTCTCACCAGCATGGC |
| | CATCTCGGTGGTGTGCTTCTTCTTCCTGGTGCACTGACTGCCTGGTGCCCAGCACATGTG |
| | CTGCCCTACAGCACCCTGTGGTCTTCCTCGATAAAGGGAACCACTTTCTTATTTTTTTCT |
| | ATTTTTTTTTTTTTGTTATCCTGTATACCTCCTCCAGCCATGAAGTAGAGGACTAACCAT |
| | GTGTTATGTTTTCGAAAATCAAATGGTATCTTTTGGAGGAAGATACATTTTAGTGGTAGC |
| | ATATAGATTGTCCTTTTGCAAAGAAAGAAAAAAAACCATCAAGTTGTGCCAAATTATTCT |
| | CCTATGTTTGGCTGCTAGAACATGGTTACCATGTCTTTCTCTCTCACTCCCTCCCTTTCT |
| | ATCGTTCTCTCTTTGCATGGATTTCTTTGAAAAAAAATAAATTGCTCAAATAAAAA |
| GPC3 (isoform | SEQ ID NO: 14 |
| P51654-1): | AGGTAGCTGCGAGGAAACTTTTGCAGCGGCTGGGTAGCAGCACGTCTCTTGCTCCTCAGG |
| NM_004484.4 | GCCACTGCCAGGCTTGCCGAGTCCTGGGACTGCTCTCGCTCCGGCTGCCACTCTCCCGCG |
| | CTCTCCTAGCTCCCTGCGAAGCAGGATGGCCGGGACCGTGCGCACCGCGTGCTTGGTGGT |
| | GGCGATGCTGCTCAGCTTGGACTTCCCGGGACAGGCGCAGCCCCCGCCGCCGCCGCCGGA |
| | CGCCACCTGTCACCAAGTCCGCTCCTTCTTCCAGAGACTGCAGCCCGGACTCAAGTGGGT |
| | GCCAGAAACTCCCGTGCCAGGATCAGATTTGCAAGTATGTCTCCCTAAGGGCCCAACATG |
| | CTGCTCAAGAAAGATGGAAGAAAAATACCAACTAACAGCACGATTGAACATGGAACAGCT |
| | GCTTCAGTCTGCAAGTATGGAGCTCAAGTTCTTAATTATTCAGAATGCTGCGGTTTTCCA |
| | AGAGGCCTTTGAAATTGTTGTTCGCCATGCCAAGAACTACACCAATGCCATGTTCAAGAA |
| | CAACTACCCAAGCCTGACTCCACAAGCTTTTGAGTTTGTGGGTGAATTTTTCACAGATGT |
| | GTCTCTCTACATCTTGGGTTCTGACATCAATGTAGATGACATGGTCAATGAATTGTTTGA |
| | CAGCCTGTTTCCAGTCATCTATACCCAGCTAATGAACCCAGGCCTGCCTGATTCAGCCTT |
| | GGACATCAATGAGTGCCTCCGAGGAGCAAGACGTGACCTGAAAGTATTTGGGAATTTCCC |
| | CAAGCTTATTATGACCCAGGTTTCCAAGTCACTGCAAGTCACTAGGATCTTCCTTCAGGC |
| | TCTGAATCTTGGAATTGAAGTGATCAACACAACTGATCACCTGAAGTTCAGTAAGGACTG |
| | TGGCCGAATGCTCACCAGAATGTGGTACTGCTCTTACTGCCAGGGACTGATGATGGTTAA |
| | ACCCTGTGGCGGTTACTGCAATGTGGTCATGCAAGGCTGTATGGCAGGTGTGGTGGAGAT |
| | TGACAAGTACTGGAGAGAATACATTCTGTCCCTTGAAGAACTTGTGAATGGCATGTACAG |
| | AATCTATGACATGGAGAACGTACTGCTTGGTCTCTTTTCAACAATCCATGATTCTATCCA |
| | GTATGTCCAGAAGAATGCAGGAAAGCTGACCACCACTATTGGCAAGTTATGTGCCCATTC |
| | TCAACAACGCCAATATAGATCTGCTTATTATCCTGAAGATCTCTTTATTGACAAGAAAGT |
| | ATTAAAAGTTGCTCATGTAGAACATGAAGAAACCTTATCCAGCCGAAGAAGGGAACTAAT |
| | TCAGAAGTTGAAGTCTTTCATCAGCTTCTATAGTGCTTTGCCTGGCTACATCTGCAGCCA |
| | TAGCCCTGTGGCGGAAAACGACACCCTTTGCTGGAATGGACAAGAACTCGTGGAGAGATA |
| | CAGCCAAAAGGCAGCAAGGAATGGAATGAAAAACCAGTTCAATCTCCATGAGCTGAAAAT |
| | GAAGGGCCCTGAGCCAGTGGTCAGTCAAATTATTGACAAACTGAAGCACATTAACCAGCT |
| | CCTGAGAACCATGTCTATGCCCAAAGGTAGAGTTCTGGATAAAAACCTGGATGAGGAAGG |
| | GTTTGAAAGTGGAGACTGCGGTGATGATGAAGATGAGTGCATTGGAGGCTCTGGTGATGG |
| | AATGATAAAAGTGAAGAATCAGCTCCGCTTCCTTGCAGAACTGGCCTATGATCTGGATGT |
| | GGATGATGCGCCTGGAAACAGTCAGCAGGCAACTCCGAAGGACAACGAGATAAGCACCTT |
| | TCACAACCTCGGGAACGTTCATTCCCCGCTGAAGCTTCTCACCAGCATGGCCATCTCGGT |
| | GGTGTGCTTCTTCTTCCTGGTGCACTGACTGCCTGGTGCCCAGCACATGTGCTGCCCTAC |
| | AGCACCCTGTGGTCTTCCTCGATAAAGGGAACCACTTTCTTATTTTTTTCTATTTTTTTT |
| | TTTTTGTTATCCTGTATACCTCCTCCAGCCATGAAGTAGAGGACTAACCATGTGTTATGT |
| | TTTCGAAAATCAAATGGTATCTTTTGGAGGAAGATACATTTTAGTGGTAGCATATAGATT |
| | GTCCTTTTGCAAAGAAAGAAAAAAAACCATCAAGTTGTGCCAAATTATTCTCCTATGTTT |
| | GGCTGCTAGAACATGGTTACCATGTCTTTCTCTCTCACTCCCTCCCTTTCTATCGTTCTC |
| | TCTTTGCATGGATTTCTTTGAAAAAAAATAAATTGCTCAAATAAAAA |
| GPC4: | SEQ ID NO: 15 |
| NM_001448.3 | AGTCAATCAGCGGCAGAGCTGAGAAGGGCACAGCCGCGCGGCGGAGGGCAGAGTCAGCCG |
| | AGCCGAGTCCAGCCGGACGAGCGGACCAGCGCAGGGCAGCCCAAGCAGCGCGCAGCGAAC |
| | GCCCGCCGCCGCCCACACCCTCTGCGGTCCCCGCGGCGCCTGCCACCCTTCCCTCCTTCC |
| | CCGCGTCCCCGCCTCGCCGGCCAGTCAGCTTGCCGGGTTCGCTGCCCCGCGAAACCCCGA |
| | GGTCACCAGCCCGCGCCTCTGCTTCCCTGGGCCGCGCGCCGCCTCCACGCCCTCCTTCTC |
| | CCCTGGCCCGGCGCCTGGCACCGGGGACCGTTGCCTGACGCGAGGCCCAGCTCTACTTTT |
| | CGCCCCGCGTCTCCTCCGCCTGCTCGCCTCTTCCACCAACTCCAACTCCTTCTCCCTCCA |
| | GCTCCACTCGCTAGTCCCCGACTCCGCCAGCCCTCGGCCCGCTGCCGTAGCGCCGCTTCC |
| | CGTCCGGTCCCAAAGGTGGGAACGCGTCCGCCCCGGCCCGCACCATGGCACGGTTCGGCT |
| | TGCCCGCGCTTCTCTGCACCCTGGCAGTGCTCAGCGCCGCGCTGCTGGCTGCCGAGCTCA |
| | AGTCGAAAAGTTGCTCGGAAGTGCGACGTCTTTACGTGTCCAAAGGCTTCAACAAGAACG |
| | ATGCCCCCCTCCACGAGATCAACGGTGATCATTTGAAGATCTGTCCCCAGGGTTCTACCT |
| | GCTGCTCTCAAGAGATGGAGGAGAAGTACAGCCTGCAAAGTAAAGATGATTTCAAAAGTG |
| | TGGTCAGCGAACAGTGCAATCATTTGCAAGCTGTCTTTGCTTCACGTTACAAGAAGTTTG |
| | ATGAATTCTTCAAAGAACTACTTGAAAATGCAGAGAAATCCCTGAATGATATGTTTGTGA |
| | AGACATATGGCCATTTATACATGCAAAATTCTGAGCTATTTAAAGATCTCTTCGTAGAGT |
| | TGAAACGTTACTACGTGGTGGGAAATGTGAACCTGGAAGAAATGCTAAATGACTTCTGGG |
| | CTCGCCTCCTGGAGCGGATGTTCCGCCTGGTGAACTCCCAGTACCACTTTACAGATGAGT |
| | ATCTGGAATGTGTGAGCAAGTATACGGAGCAGCTGAAGCCCTTCGGAGATGTCCCTCGCA |
| | AATTGAAGCTCCAGGTTACTCGTGCTTTTGTAGCAGCCCGTACTTTCGCTCAAGGCTTAG |
| | CGGTTGCGGGAGATGTCGTGAGCAAGGTCTCCGTGGTAAACCCCACAGCCCAGTGTACCC |
| | ATGCCCTGTTGAAGATGATCTACTGCTCCCACTGCCGGGGTCTCGTGACTGTGAAGCCAT |
| | GTTACAACTACTGCTCAAACATCATGAGAGGCTGTTTGGCCAACCAAGGGGATCTCGATT |
| | TTGAATGGAACAATTTCATAGATGCTATGCTGATGGTGGCAGAGAGGCTAGAGGGTCCTT |
| | TCAACATTGAATCGGTCATGGATCCCATCGATGTGAAGATTTCTGATGCTATTATGAACA |
| | TGCAGGATAATAGTGTTCAAGTGTCTCAGAAGGTTTTCCAGGGATGTGGACCCCCCAAGC |
| | CCCTCCCAGCTGGACGAATTTCTCGTTCCATCTCTGAAAGTGCCTTCAGTGCTCGCTTCA |
| | GACCACATCACCCCGAGGAACGCCCAACCACAGCAGCTGGCACTAGTTTGGACCGACTGG |
| | TTACTGATGTCAAGGAGAAACTGAAACAGGCCAAGAAATTCTGGTCCTCCCTTCCGAGCA |
| | ACGTTTGCAACGATGAGAGGATGGCTGCAGGAAACGGCAATGAGGATGACTGTTGGAATG |
| | GGAAAGGCAAAAGCAGGTACCTGTTTGCAGTGACAGGAAATGGATTAGCCAACCAGGGCA |
| | ACAACCCAGAGGTCCAGGTTGACACCAGCAAACCAGACATACTGATCCTTCGTCAAATCA |
| | TGGCTCTTCGAGTGATGACCAGCAAGATGAAGAATGCATACAATGGGAACGACGTGGACT |
| | TCTTTGATATCAGTGATGAAAGTAGTGGAGAAGGAAGTGGAAGTGGCTGTGAGTATCAGC |
| | AGTGCCCTTCAGAGTTTGACTACAATGCCACTGACCATGCTGGGAAGAGTGCCAATGAGA |
| | AAGCCGACAGTGCTGGTGTCCGTCCTGGGGCACAGGCCTACCTCCTCACTGTCTTCTGCA |
| | TCTTGTTCCTGGTTATGCAGAGAGAGTGGAGATAATTCTCAAACTCTGAGAAAAAGTGTT |
| | CATCAAAAAGTTAAAAGGCACCAGTTATCACTTTTCTACCATCCTAGTGACTTTGCTTTT |
| | TAAATGAATGGACAACAATGTACAGTTTTTACTATGTGGCCACTGGTTTAAGAAGTGCTG |
| | ACTTTGTTTTCTCATTCAGTTTTGGGAGGAAAAGGGACTGTGCATTGAGTTGGTTCCTGC |
| | TCCCCCAAACCATGTTAAACGTGGCTAACAGTGTAGGTACAGAACTATAGTTAGTTGTGC |
| | ATTTGTGATTTTATCACTCTATTATTTGTTTGTATGTTTTTTTCTCATTTCGTTTGTGGG |
| | TTTTTTTTTCCAACTGTGATCTCGCCTTGTTTCTTACAAGCAAACCAGGGTCCCTTCTTG |
| | GCACGTAACATGTACGTATTTCTGAAATATTAAATAGCTGTACAGAAGCAGGTTTTATTT |
| | ATCATGTTATCTTATTAAAAGAAAAAGCCCAAAAAGCAGTAAAATTTCCATTTCTCCCTG |
| | TTATTTTAGTTGCCTTATCTGGAGAGACGTGGAGGTGATTTTCTTTTTTTTAAATTATTA |
| | TTAAGACAGAATGTGAGGGCACAAGCAGGCTTCTGAGCCACTTGTCAGATTGTATTCAAA |
| | GCATCAATCCAAGAAGGAGGTTATGTGTACTTCATTTATTGGTGATAGTTGGAAGAGACT |
| | GCAGACTACTGCTTTGAATGAGTTGAATTACATAAGCTAAGATCACTATAGGTCCATTTC |
| | TTGAACCCACTTATACATAAAATGTAACCCATTTAGAAAAAGATTCTGGATATCATCCCC |
| | CTTGAAAGATAGAAAGCATTCAGGATGTCCCAGTTATCACATGTTCACACTTGGGTTTAG |
| | GGGTGTTTTTTTTTAAAACCAGGCAGGTTAGCTAGCCCACCCTGTGCTAGTTTTCATGTT |
| | CACACTGACCCTATTTGAATTAATATCCTTTGTTAGAGTGGTCGAGATTTCAAACCCAAT |
| | TATGTACAGGGAGCTGTCTGAGAGCTAGCCAGAACTGGGGTACAGCCTGGGCTCAGGGAA |
| | TAGCTGTCAACACTCGGGCAAAGTTTTTGTCTGTGCATGTGTATCTCCATTTGTTTTGGG |
| | ATCCCAGTTTTTGTTTTAAGAGAGTATAAGGTGTCTCATTTGAGTCTTTTTCTTACCTAG |
| | CCCCCTCTTATCAGTAAAACAAAGGACTTGCCATGGTTCACAGCAATGTGCTACGATCCA |
| | AGATATCAGCCAAGGAGCCCACTTAGGGGAGAACTAGGTGTCCAGATTTTTGTATGTGTT |
| | GTTTTTCTTGGGGGATGGGGTGGGGTGGGAGTAGGTAGAGCTGAGAATACTACATCTTAG |
| | TGGTGACCTTTAGCCACGTGGGTGAAGTGGCAAAGGCCATGGCCATATCTGTTGTCCCAG |
| | GCCAAAGACTAACAACTGCCTTGGGAATCCCTTCCTTGTGTCCTTACCAAATGATAGCTC |
| | ATAAAACTCTGATAATGTAACAAATCACTTTCAAAGGAGTTCCCAGAAGTCTTCAGAAAG |
| | ACTAAAATTCTGTCTCTTCCTGCTTTAGACAGCCATTAAGATCCCAACTAATTTTACCGA |
| | ACCTAAAACCCACAAAGAGGTTGTTTGTGTTATTGTTCAATCTTCAGTTGTAAGAGTAAT |
| | TCTCTATTTTTATATTGAAACATAATTACTTGATAGCTCAGGGTCTACATTTCATTCAAC |
| | TTTTTACACCAAATTCTGCAGAGTGGTCAAAATGGAATATTGGGGGCTGTTGTAAACAGA |
| | GGCTTAATTTTATTAGAAGTAGCCAGTTATTTATTAAAGCATGATGTTAATAAAATAGGC |
| | ATATTCTGGCTGGTGTGTATAAGTGGTTTTTTTTTTAAAACAAATCAGAAGACATAAAAT |
| | TGAAAAGGCACTCCATTTATATATAAGGGAAAATAGATTGTAAATAGCTAAGGGGCCTTG |
| | TGAGAACTTGGAAAATGTGCTGATACTTAGTAACTACCTCAGTTGTGAGGAATTTCCTTT |
| | TTAGCTTAGACTGCTATTACTTTTACTTGGTAGAAATGAATCTGGTCTCCAAAATATGGC |
| | TCAGAGAGTTATCCCCATAGACCTTGTTAGATTGTTTTTAATTTAAAACCTTGAACTCTG |
| | CCCAGCTGTTTTCTTCCAGTCAAATTCTCTAAACCAATAACATTTTCCAAATTAAGTATT |
| | CTTGAAGGTTCTTCTGAACTCTTACTGATTTGTTTTCTTTCTTTGCATTTTTTCCTGTTA |
| | CTCTTTTATTATTTAAATTGTCTCTTGCTTTTGAACTTCAACTATGAAATAAACAAATCA |
| | TAGATTTTTCCATTCAGGTCTGACTGGATAACTCTTGGCTTCTAAGCAGCTGGCAAGCTC |
| | ACCCCCACTCCCCAAAAAAGAAGAAAGGCAAGTTGAATATTTTAAAACCTTAAGAGTTTG |
| | GAGTATGAAGTAGAATAAAAGCCTTGTTAGGAGAAGACCTGACTTTTCCAAAGTTAACAG |
| | CATGTGAAGGGCTTGTGTTTTACAAAGTCATTTATAAATTATTTTTCTTTAAAACACATC |
| | ACCTACTCTGTACCTTAAAGATTTTACAACTCTTAGATTTATATTTTATTTCAAAAATCG |
| | TAACTAGCACCTGTTATTTTGATTAAAAGACACTGCGAAGAATTGACGTAAATTTCTAAA |
| | GTTTAGTTACTGAGTGTTCAAAACCTAACAGTTCACATGAAGTAAAAATTGTCATTTGTG |
| | ATTACAGAATCAAAATAAAAATAGGTATGAATGGTGCTA |
| GPC5: | SEQ ID NO: 16 |
| NM_004466.6 | ATCCCAGGTTAGCTGCTGCGAGCCGAGCCGGGCGGCGGAGGCGGCGGCGGCGGCGGCAGT |
| | GGCGGCAGTGGCGGCAGTGGCGGCAGCGGCAGCAGTTGCAGCAGTGGTGGCCAGAGCGGA |
| | TGCTTGCGGGCTCCCTGCGGCTCCACTAGTTTTCTTCGCCCCGCCCAGCCGCCCACTCTT |
| | CTCGGCTAGGGAAGAAGACCAGAGGGTGCTCAGCTGGAAAACTCTGGTGTCTCAGCTTAG |
| | GGCCTCCTCCGGGAAGAGCTAACTGCTCCCAGGTGAAGCCGGTGCCCGCGGGCGGTCCGT |
| | ACACCCCGCAGCCGGCTCGCACCGCTCGAGAGCCTCGGCCGCTGTGTCTTCCACGTCTGC |
| | AGCTCAGCCAGGGCGCGCAGGGCGAGTGGGGTCCACTGGCGGGTAAAGGGGACCAGGACG |
| | GCGAGGATGGACGCACAGACCTGGCCCGTGGGCTTTCGCTGCCTCCTCCTTCTGGCCCTG |
| | GTTGGGTCCGCCCGCAGCGAGGGCGTGCAGACCTGCGAAGAAGTTCGGAAACTTTTCCAG |
| | TGGCGGCTGCTGGGAGCTGTCAGGGGGCTGCCGGATTCGCCGCGGGCAGGACCTGATCTT |
| | CAGGTTTGCATATCCAAAAAGCCTACATGTTGCACCAGGAAGATGGAGGAGAGATATCAG |
| | ATTGCGGCTCGCCAGGATATGCAGCAGTTTCTTCAAACGTCCAGCTCTACATTAAAGTTT |
| | CTAATATCTCGAAATGCGGCTGCTTTTCAAGAAACCCTTGAAACTCTCATCAAACAAGCA |
| | GAAAATTACACCAGTATACTTTTTTGCAGTACCTACAGGAACATGGCCTTGGAGGCTGCT |
| | GCTTCGGTTCAGGAGTTCTTCACTGATGTGGGGCTGTATTTATTTGGTGCGGATGTTAAT |
| | CCTGAAGAATTTGTAAACAGATTTTTTGACAGTCTTTTTCCTCTGGTCTACAACCACCTC |
| | ATTAACCCTGGTGTGACTGACAGTTCCCTGGAATACTCAGAATGCATCCGGATGGCTCGC |
| | CGGGATGTGAGTCCATTTGGTAATATTCCCCAAAGAGTAATGGGACAGATGGGGAGGTCC |
| | CTGCTGCCCAGCCGCACTTTTCTGCAGGCACTCAATCTGGGCATTGAAGTCATCAACACC |
| | ACAGACTATCTGCACTTCTCCAAAGAGTGCAGCAGAGCCCTCCTGAAGATGCAATACTGC |
| | CCGCACTGCCAAGGCCTGGCGCTCACTAAGCCTTGTATGGGATACTGCCTCAATGTCATG |
| | CGAGGCTGCCTGGCGCACATGGCGGAGCTTAATCCACACTGGCATGCATATATCCGGTCG |
| | TTGGAAGAACTCTCGGATGCAATGCATGGAACATACGACATTGGACACGTGCTGCTGAAC |
| | TTTCACTTGCTTGTTAATGATGCTGTGTTACAGGCTCACCTCAATGGACAAAAATTATTG |
| | GAACAGGTAAATAGGATTTGTGGCCGCCCTGTAAGAACACCCACACAAAGCCCCCGTTGT |
| | TCTTTTGATCAGAGCAAAGAGAAGCATGGAATGAAGACCACCACAAGGAACAGTGAAGAG |
| | ACGCTTGCCAACAGAAGAAAAGAATTTATCAACAGCCTTCGACTGTACAGGTCATTCTAT |
| | GGAGGTCTAGCTGATCAGCTTTGTGCTAATGAATTAGCTGCTGCAGATGGACTTCCCTGC |
| | TGGAATGGAGAAGATATAGTAAAAAGTTATACTCAGCGTGTGGTTGGAAATGGAATCAAA |
| | GCCCAGTCTGGAAATCCTGAAGTCAAAGTCAAAGGAATTGATCCTGTGATAAATCAGATT |
| | ATTGATAAACTGAAGCATGTTGTTCAGTTGTTACAGGGTAGATCACCCAAACCTGACAAG |
| | TGGGAACTTCTTCAGCTGGGCAGTGGTGGAGGCATGGTTGAACAAGTCAGTGGGGACTGT |
| | GATGATGAAGATGGTTGCGGGGGATCAGGAAGTGGAGAAGTCAAGAGGACACTGAAGATC |
| | ACAGACTGGATGCCAGATGATATGAACTTCAGTGATGTAAAGCAAATCCATCAAACAGAC |
| | ACTGGCAGTACTTTAGACACAACAGGAGCAGGATGTGCAGTGGCGACTGAATCTATGACA |
| | TTCACTCTGATAAGTGTGGTGATGTTACTTCCCGGGATTTGGTAACTGAACTCTTCTGTC |
| | CTGACATACCTTACTGAAGTCTCGATTTCTTCTCTCTCTGCATATGCCTGGAATAAGAGA |
| | TCCTTTTTCAATGTAACAATTATATTTATGAAAAGATATGTTACACTAACTTCTCAGAAG |
| | CCAAGCTGAAATATTCATAAAGTCCCTAAAACTCAACGTTTAAATGACACACTTTAAAAA |
| | TATGTCTTTTTTCAATCTAACTGAAAACCTTCTTAACTTCTAATATATTAAATCTGAAGA |
| | TGTGAAGGGCACAGAAGTGACTTTGAATAAGAAGAATTTAGTGTATCTGTAATTTTATTA |
| | TCAATTCCAAGCCCCTTCCTTTCTAAATTAAAAATGTTTTCATTTGAAAGTGTATTTGCC |
| | AGACAATGAAAACAGTATGCAGTATTTCTTAAAGTATTGAAATTAGAATATCATGAAATA |
| | AATCAAAACATACAATGGCAAGTAGTATGCATGCATATTCAAGAGACTCTTCCATTTTTG |
| | CAAGCTGTAGAAGGAAATGTCTGAATGTCTATAAGTTATGGGGTAGATTCTTGAGAAGCA |
| | TTTCATATAATTTCACTGAAGAACCTTGATAATTTTGACCCACTGTAACTTAGCCACTGA |
| | TGAACCTTAAAGCTGAGTATTTTATTAACACCTGATTTGTATTCCATTATATTCAAAATG |
| | CATCTTTGGTATTGTGCCTCTGCTCCCATCTCTCTCTTTGCCTCATAGATTTAGCTATGT |
| | TGGGAAGCACATGCTTGCTCTAGGAATATCTCCAATAAAGCTGTTAACTATTTGGTGGAA |
| | AAA |
| GPC6: | SEQ ID NO: 17 |
| NM_005708.5 | CCTTTCTCCTTCCCTCTTGCCTCCAGTGACTGTCTCCAGGATTTCTCTCTTCCTATTTCA |
| | GGAGGACTCTCACAGGCTCCCACAGCCTGTGTTAAGCTGAGGTTTCCCCTAGATCTCGTA |
| | TATCCCCAACACATACCTCCACGCACACACATCCCCAAGAACCTCGAGCTCACACCAACA |
| | GACACACGCGCGCATACACACTCGCTCTCGCTTGTCCATCTCCCTCCCGGGGGAGCCGGC |
| | GCGCGCTCCCACCTTTGCCGCACACTCCGGCGAGCCGAGCCCGCAGCGCTCCAGGATTCT |
| | GCGGCTCGGAACTCGGATTGCAGCTCTGAACCCCCATGGTGGTTTTTTAAACACTTCTTT |
| | TCCTTCTCTTCCTCGTTTTGATTGCACCGTTTCCATCTGGGGGCTAGAGGAGCAAGGCAG |
| | CAGCCTTCCCAGCCAGCCCTTGTTGGCTTGCCATCGTCCATCTGGCTTATAAAAGTTTGC |
| | TGAGCGCAGTCCAGAGGGCTGCGCTGCTCGTCCCCTCGGCTGGCAGAAGGGGGTGACGCT |
| | GGGCAGCGGCGAGGAGCGCGCCGCTGCCTCTGGCGGGCTTTCGGCTTGAGGGGCAAGGTG |
| | AAGAGCGCACCGGCCGTGGGGTTTACCGAGCTGGATTTGTATGTTGCACCATGCCTTCTT |
| | GGATCGGGGCTGTGATTCTTCCCCTCTTGGGGCTGCTGCTCTCCCTCCCCGCCGGGGCGG |
| | ATGTGAAGGCTCGGAGCTGCGGAGAGGTCCGCCAGGCGTACGGTGCCAAGGGATTCAGCC |
| | TGGCGGACATCCCCTACCAGGAGATCGCAGGGGAACACTTAAGAATCTGTCCTCAGGAAT |
| | ATACATGCTGCACCACAGAAATGGAAGACAAGTTAAGCCAACAAAGCAAACTCGAATTTG |
| | AAAACCTTGTGGAAGAGACAAGCCATTTTGTGCGCACCACTTTTGTGTCCAGGCATAAGA |
| | AATTTGACGAATTTTTCCGAGAGCTCCTGGAGAATGCAGAAAAGTCACTAAATGATATGT |
| | TTGTACGGACCTATGGCATGCTGTACATGCAGAATTCAGAAGTCTTCCAGGACCTCTTCA |
| | CAGAGCTGAAAAGGTACTACACTGGGGGTAATGTGAATCTGGAGGAAATGCTCAATGACT |
| | TTTGGGCTCGGCTCCTGGAACGGATGTTTCAGCTGATAAACCCTCAGTATCACTTCAGTG |
| | AAGACTACCTGGAATGTGTGAGCAAATACACTGACCAGCTCAAGCCATTTGGAGACGTGC |
| | CCCGGAAACTGAAGATTCAGGTTACCCGCGCCTTCATTGCTGCCAGGACCTTTGTCCAGG |
| | GGCTGACTGTGGGCAGAGAAGTTGCAAACCGAGTTTCCAAGGTCAGCCCAACCCCAGGGT |
| | GTATCCGTGCCCTCATGAAGATGCTGTACTGCCCATACTGTCGGGGGCTTCCCACTGTGA |
| | GGCCCTGCAACAACTACTGTCTCAACGTCATGAAGGGCTGCTTGGCAAATCAGGCTGACC |
| | TCGACACAGAGTGGAATCTGTTTATAGATGCAATGCTCTTGGTGGCAGAGCGACTGGAGG |
| | GGCCATTCAACATTGAGTCGGTCATGGACCCGATAGATGTCAAGATTTCTGAAGCCATTA |
| | TGAACATGCAAGAAAACAGCATGCAGGTGTCTGCAAAGGTCTTTCAGGGATGTGGTCAGC |
| | CCAAACCTGCTCCAGCCCTCAGATCTGCCCGCTCAGCTCCTGAAAATTTTAATACACGTT |
| | TCAGGCCCTACAATCCTGAGGAAAGACCAACAACTGCTGCAGGCACAAGCTTGGACCGGC |
| | TGGTCACAGACATAAAAGAGAAATTGAAGCTCTCTAAAAAGGTCTGGTCAGCATTACCCT |
| | ACACTATCTGCAAGGACGAGAGCGTGACAGCGGGCACGTCCAACGAGGAGGAATGCTGGA |
| | ACGGGCACAGCAAAGCCAGATACTTGCCTGAGATCATGAATGATGGGCTCACCAACCAGA |
| | TCAACAATCCCGAGGTGGATGTGGACATCACTCGGCCTGACACTTTCATCAGACAGCAGA |
| | TTATGGCTCTCCGTGTGATGACCAACAAACTAAAAAACGCCTACAATGGCAATGATGTCA |
| | ATTTCCAGGACACAAGTGATGAATCCAGTGGCTCAGGGAGTGGCAGTGGGTGCATGGATG |
| | ACGTGTGTCCCACGGAGTTTGAGTTTGTCACCACAGAGGCCCCCGCAGTGGATCCCGACC |
| | GGAGAGAGGTGGACTCTTCTGCAGCCCAGCGTGGCCACTCCCTGCTCTCCTGGTCTCTCA |
| | CCTGCATTGTCCTGGCACTGCAGAGACTGTGCAGATAATCTTGGGTTTTTGGTCAGATGA |
| | AACTGCATTTTAGCTATCTGAATGGCCAACTCACTTCTTTTCTTACACTCTTGGACAATG |
| | GACCATGCCACAAAAACTTACCGTTTTCTATGAGAAGAGAGCAGTAATGCAATCTGCCTC |
| | CCTTTTTGTTTTCCCAAAGAGTACCGGGTGCCAGACTGAACTGCTTCCTCTTTCCTTCAG |
| | CTATCTGTGGGGACCTTGTTTATTCTAGAGAGAATTCTTACTCAAATTTTTCGTACCAGG |
| | AGATTTTCTTACCTTCATTTGCTTTTATGCTGCAGAAGTAAAGGAATCTCACGTTGTGAG |
| | GGTTTTTTTTTTCTCATTTAAAATAAAAAAGGAAGAAAGAAAATAATTTTCCTTGTAAAA |
| | TCGGGCCAAACCCCAAGACAGCTACATTTTCAACAAAAAAGCAAACAGAGAAAAATAAAT |
| | GAACTTTAACACTGTAAGTTCAGCATTGACAGCCAACTTCCAGTGTAAGCTTTTTTGTGA |
| | CAAATCCGGGTTTAAAAATGCTTATGGGGAGAAAGCTTAAAGTTTGTTCTAAATGTAGTG |
| | AAAATGCACTGTTGTCTTGGAGGTATCATCTTGCACTCTAACCAAACAACACCAAATTAA |
| | TCTGGAGGTACTGATACTAAATTTGGAATCCCGTCTTTAATATGAGAACACAAATTACTA |
| | CAACTCAGTACCTCTGTCTTACTCTGCCCAATTTCAATTTAGTAATGGCACCATTATTCT |
| | TAATTCCCTTAATGTCTGTCCGCTGTATCCAATTGGCACAGATTTTCCCCTTCATCTTCA |
| | ATTACTAGTAGCTACCCCCATCAATTCACCTTCCTCAAGAGTCTCAATTGAAAATAATCT |
| | TTGATGATACAATCTCTGCTTTTGCCACAGGTTTGACATTAACTAGACGTTTCTGCCCCT |
| | GTGCTCATTACCCTCCCAAAACAATAGTTTTTTTAATGAATTGAAACACAATTAGATCCA |
| | TTCCTGAGTTTTCATTTTTAACTATTTACTTTGTGTCAGTTGGTTTCTGCATCACAAGGG |
| | CATTCTCTTACAAAACAACTCACAACAGAAAAAAAAAGTCAAGACAAAAAATATATATAT |
| | ATAGTATTGACATGGAGATTTCTTTCACTTTTTTAGTCTTAGTATGATCATCTATTTTTA |
| | TATCTATGTATATATAAATCACAGATTTTAACTTCTTAATTCCAAGCTCAGGGTTGACAC |
| | ACCTTTGTAAGAAAATGTTTTGTCAACCAGCTCTTCTTGTTTTGTGCTGCTCAAAGAAGG |
| | GATTCCTCAGTGATCGGTGAGCACCAAGAATCAAGAAAGAGAACTTTTTATGTATCTAAC |
| | TGTCCATTTATTAAAAATTTGCCAGGGCACACCCTTCTGCATGAGTGTGCTTCAGCCTGG |
| | GTGCTCCAGACTACACCAAACTCATGGGATGAGCCATTGGTGTGCAGGGCTTTACCGGTG |
| | ATGGAAACTGTATGTGTAAAGAATGTTATTCATGAAAAACCATTGCTTTCATGTAAAACA |
| | CCTTCGGCCTAGGATTCCTGCCTGGCACTTGGATACATTACCTAACTCAATAGGCCAGTT |
| | TAGAGCCACAGCCTAACCTGTTCACCAAGTAGGCCAAAGCCAGGCCTTTCGCTCCCCCTG |
| | ACAGAGAGAAGCAAAATAAACCCAACTGGGGGCTTTATGAATACAGTTGGCTATCCATAT |
| | GTTTCTTAGTTTTTATGATGCATCTGGAGTAGGGAAAAGTAATGTTTCAAGACACCATAG |
| | TAGGCCAGTTACATTTTAATTAACTCATCGACAGTATTGACACGTAAATGGTATTTTTCA |
| | ATCTGTTCTCATCAAAATTAAAATTAGTTATTGATTTAGTCATCACATTTAAGTAGAACA |
| | CATGTGGCACAATAGTATTAAGTATATTTGAGCACTTTTGCAAGACAGAAAGTATTTACA |
| | AGCATCTATTGCTACTTTATGCCTAATGAAATATAAAATGTAGGATGATGAGAGACTTTG |
| | TGGCATTCTTTCATTTAAACCTTTATGAAATTAACCATCTAGTTCATCATTACATACTCC |
| | TAGAAAACCATCCATTTCACTGCCCACATTTTGGCTTCCTACCACAAATTCCACATAAGA |
| | CTTCTCTATGAAGGACCAGTCATTTACATTGCTCATTTCATTCTGTGTTCAGAAGTCAGT |
| | ATTGTCTTTCTCCAATAATTAAGGTGCAATACAAATTAAATCAACCTTGATTTTCCAGAC |
| | TATTTGAATAATAACTTGCTTTTATGGCTTTAAACTCCAGGTCTTTCCTTTTTAAAATAT |
| | TTCTGCATTCTCTTTTATGAACTAGATCAGGATGCCTTGTTTTTCCTGTGCTTTCACCCC |
| | CAAAAGGTTTTGTGCATGTGTATGAATACACATAATATTAGTGAATGTGAACCCATGTTC |
| | TATACGCACTAAACACACAGTTAATATATAAACGCCTGCTTCATTTGTTTCAACACCTTC |
| | ATTATCAATATGATTCATATTGATGAGATATAGTGATGCATAATGAGCCAGGCAGAGAGC |
| | TGAAATGATATCAGTTAATGATGTCACAGTGTTAGCACCCTGCATGGATGTGGTCAAAAT |
| | ATCATTCAAGTTGTACTTCCTAGTTTTTATCATCATACTACTATGTCATATGATTTTCAG |
| | ACTAATTTAACATGAGAAATGTTTTGGCCCTAAAGGATTTCACTTAACCTAGTTACCCTA |
| | TTTCAGTAAGTATTTTGGAATTCAACCCTCGAATTATTTTTTCTCATTTCAGCATAGTGA |
| | TAGGGGATGCAATGAGGCTTCATTATTTTTTATGACCTGCCCCTCATTTGCTCTGATGTT |
| | CCCTAAATTCTGTAATCATATCATAACTTTTGTTATGAATAGAGAGGAATGGGCTCACTG |
| | AAACCTGACACTAGAAATTGGTGGGTGATGCTCATAACTGCAAACACTTAGCTTATTGAA |
| | GTGCCTCTATTTACATGTTCTTTAGTTATAATATGTATTTTTCTAACAGAAATACACGTC |
| | TGTAATTGGTATATATTATACTTTGTATGTGTCACAACAAAAGCTAAACAGAGGCTAAAG |
| | TCTTTAGCAGAGAAGAATGAATTACAAATTTACCAATAAGAGCTCTGTTTATGGTTCTGT |
| | ATTGAAGGACAACCATGACAACCTTCTGTCATTTTCACGAAGGCCAAGTTGGTACTTTGA |
| | TTATAGCCATCGTAGAACATTAGCACTAGAAATCTGTCTCCCTTACAATGACTTACCTAG |
| | CTAGCATCAAGTAACTTGTATCACCTGCTTTAAATGTAAGGGATAGAAAATACTATTTTG |
| | TCTACACTGAAAAACATTATTATTCATTGATAAGTAGTAATAATTATAAAAGTAACATTC |
| | TTTAAAGCAAACACATTGAAAATATTATTACAGGCAGAACCCTGATTTTTAATTTATATT |
| | CCCTGCTGCAGACATTAGCCTTCTTTAATTCTTTTGCCATTACATACATGTTTCGGCTAC |
| | AGACTGAACACGAACTAAAGTCCCTAAGGAGGAAAAAACCTTAACATATTCCTGAGATGT |
| | GATTCTTTTATTACTTCTTTTTGCAAAGCTAATTATCAAATGATTTGATTAAACCTAGAC |
| | GCATTAGGAAATGCAAGTTTTACCTAAAACTTGCAGAAATTTAAACACATTTCCATTTAC |
| | TTACTAACGAACAGACCTGATTTTTATTTAAAAACACAATTTAGCTTTGCCAACAGAAGA |
| | GTAAAGGAAACCATAACTTATAAATTTGTAATGTGTTCCTCCTTGTTGGAGACATTAGCC |
| | AGTGTTTAGTTTTAGACCAACTTATACAAAGAGAGTATTGATTTGTACACATTCTGGAGA |
| | CTTGCTATATCATGGTTTGCTATGGCACAGACTCAGCTTAGCATGGGGACATTTTGAACT |
| | TCATTCAAAGTTGAGTAGTTACTGGAACCTTTGACATTGCCTTGTAATGAGGTACTTCCA |
| | AAAAAAGACCCCTAACAATGGCATAATAGTGAGGTCTCTCTGTGCATATACATAATATAT |
| | ATGCAGGATAAATGTGTGCACCAATCAAATCTAAAATTTTATGTGACTGTCAAATGAATA |
| | TTATTTGGGTTAAGATTTTTCATTTCTGATTTGGATAGAAAATTGCTATTAATCTTGTAT |
| | TAAAATAGTTTTTAAAAATCTACCAGTGCCCTTTCTGCATTTTCTTAATTATTGTCATAG |
| | TCTAAATTTCAGAGCCATGGAATTTTTTGTGTTTGAGACATTTTTAGAAGCTTCAATATT |
| | AAAAAACAACAACAAACCTCGTATATTAAAACAGTGCCACTCTCCTAAGTACACTCAGCA |
| | TCTGAAATCAGGAACTATTTGAACTTTAGAACATTTAGAGCTTCCCAAATGTGTATGGAA |
| | AATACTGTAGCGCTGTTCTTTTGTACTGATTTCTTCTCAGAGGATAAATTTAGTGACTTG |
| | AAAACTGATGTCAATACTTTATAAATGAGGTCAGAAAATAAGATCAAATGTGAAAAGACT |
| | AATAATTTCCCAATTTAGCCAATTGCATTGATTTTTATACTTCTGAAATGTTTCCATCCT |
| | CTCAGGCATGCTAAATATTGTATAACCTGCTAAAGATTTATTTCACAAATGCTTATTGAA |
| | CTCTTATTCTCTATAAACCCTGCTCTAGGGGATTTATCACTGGCTGTGAAAAACCACCAG |
| | TTGAAAATTATTCATTCCTTAATGCAGCTAATCATAATTATTACAGATAAATAATGTATT |
| | TCAAGAATGATCTTATAAATAAAGGAACCCATACAAACAAA |

Repression of PTK7, GPC1, GPC2, GPC3, GPC4, GPC5, or GPC6 expression will preferably decrease the quantity of PTK7, GPC1, GPC2, GPC3, GPC4, GPC5, or GPC6 expression by a cell, e.g., by a fibroblast cell. For example, in a given cell the repression of PTK7, GPC1, GPC2, GPC3, GPC4, GPC5, or GPC6 by administration of a suitable nucleic acid will decrease the quantity of PTK7, GPC1, GPC2, GPC3, GPC4, GPC5, or GPC6 expressed by the cell relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function.

A role for the RNAi machinery and small RNAs in targeting of heterochromatin complexes and epigenetic gene silencing at specific chromosomal loci has been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA. RNAi based therapeutics have been progressed into Phase I, II and III clinical trials for a number of indications (Nature 2009 Jan. 22; 457 (7228): 426-433) and several RNAi based therapeutics are currently marketed as drugs approved by European and American regulatory agencies.

In the art, these RNA sequences are termed “short or small interfering RNAs” (siRNAs) or microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present invention provides the use of oligonucleotide sequences for downregulating the expression of PTK7, GPC1, GPC2, GPC3, GPC4, GPC5, or GPC6.

siRNAs are typically double stranded and, to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNAs are typically single stranded and have regions that are partially complementary enabling the ligands to form a hairpin. miRNAs are RNA genes which are transcribed from DNA but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLOS Biology, 11 (2), 1862-1879, 2004.

Typically, the siRNAs or miRNAs have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g., of one or two (ribo) nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g., vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g., of one or two (ribo) nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short, inverted repeats separated by a small loop sequence. One inverted repeat is complimentary to the gene target. In the cell the shRNA is processed by DICER into a siRNA which degrades the target gene mRNA and suppresses expression. In a preferred embodiment, the shRNA is produced endogenously (within a cell) by transcription from a vector. shRNAs may be produced within a cell by transfecting the cell with a vector encoding the shRNA sequence under control of an RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of PTK7, GPC1, GPC2, GPC3, GPC4, GPC5, or GPC6. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of PTK7, GPC1, GPC2, GPC3, GPC4, GPC5, or GPC6. In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific (e.g., heart, liver, or kidney specific) promoter. In a further embodiment, the siRNA, longer dsRNA, or miRNA is produced exogenously (in vitro) by transcription from a vector.

Suitable vectors may be oligonucleotide vectors configured to express the oligonucleotide agent capable of PTK7, GPC1, GPC2, GPC3, GPC4, GPC5, or GPC6 repression. Such vectors may be viral vectors or plasmid vectors. The therapeutic oligonucleotide may be incorporated in the genome of a viral vector and be operably linked to a regulatory sequence, e.g., promoter, which drives its expression. The term “operably linked” may include the situation where a selected nucleotide sequence and regulatory nucleotide sequence are covalently linked in such a way as to place the expression of a nucleotide sequence under the influence or control of the regulatory sequence. Thus, a regulatory sequence is operably linked to a selected nucleotide sequence if the regulatory sequence is capable of effecting transcription of a nucleotide sequence which forms part or all of the selected nucleotide sequence.

Viral vectors encoding promoter-expressed siRNA sequences are known in the art and have the benefit of long-term expression of the therapeutic oligonucleotide. Examples include lentiviral (Nature 2009 Jan. 22; 457 (7228): 426-433). adenovirus (Shen et al., FEBS Lett 2003 Mar. 27; 539 (1-3) 111-4) and retroviruses (Barton and Medzhitov PNAS Nov. 12, 2002 vol. 99, no. 23 14943-14945).

In other embodiments a vector may be configured to assist delivery of the therapeutic oligonucleotide to the site at which repression of PTK7, GPC1, GPC2, GPC3, GPC4, GPC5, or GPC6 expression is required. Such vectors typically involve complexing the oligonucleotide with a positively charged vector (e.g., cationic cell penetrating peptides, cationic polymers and dendrimers, and cationic lipids); conjugating the oligonucleotide with small molecules (e.g., cholesterol, bile acids, sugars, and lipids), polymers, antibodies or antibody-like molecules, aptamers, and RNAs; or encapsulating the oligonucleotide in nanoparticulate formulations (Wang et al., AAPS J. 2010 December; 12 (4): 492-503).

In one embodiment, a vector may comprise a nucleic acid sequence in both the sense and antisense orientation, such that when expressed as RNA the sense and antisense sections will associate to form a double stranded RNA.

Alternatively, siRNA molecules may be synthesized using standard solid or solution phase synthesis techniques which are known in the art. Linkages between nucleotides may be phosphodiester bonds or alternatives, for example, linking groups of the formula P(O) S, (thioate); P(S) S. (dithioate); P(O) NR′2; P(O)R′; P(O) OR6; CO; or CONR′2 wherein R is H (or a salt) or alkyl (1-12C) and R6 is alkyl (1-9C) is joined to adjacent nucleotides through-O-or-S—.

Modified nucleotide bases can be used in addition to the naturally occurring bases and may confer advantageous properties on siRNA molecules containing them. For example, modified bases may increase the stability of the siRNA molecule, thereby reducing the amount required for silencing. The provision of modified bases may also provide siRNA molecules which are more, or less, stable than unmodified siRNA. The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus, modified nucleotides may also include 2′substituted sugars such as 2′-O-methyl; 2′-O-methoxyethyl; 2′-O-alkyl; 2′-O-allyl: 2′-S-alkyl; 2′-S-allyl; 2′-fluoro; 2′-halo or azido-ribose. carbocyclic sugar analogues, a-anomeric sugars; epimeric sugars such as arabinose, xyloses or lyxoses, pyranose sugars, furanose sugars, and sedoheptulose. Modified nucleotides may also include phosphorodiamidate morpholino oligonucleotide (PMO).

Modified nucleotides are known in the art and include alkylated purines and pyrimidines, acylated purines and pyrimidines, and other heterocycles. These classes of pyrimidines and purines are known in the art and include pseudoisocytosine, N4, N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil,-D-mannosylqueosine, 5-methoxycarbonylmethyluracil, 5methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6, diaminopurine, methylpsuedouracil, 1-methylguanine, 1-methylcytosine.

Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001): Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498). Accordingly, the invention provides nucleic acid that is capable, when suitably introduced into or expressed within a mammalian, e.g., human, cell that otherwise expresses PTK7, GPC1, GPC2, GPC3, GPC4, GPC5, or GPC6, of suppressing PTK7, GPC1, GPC2, GPC3, GPC4, GPC5, or GPC6 expression by RNAi.

The nucleic acid may have substantial sequence identity to a portion of of DNA, pre-mRNA or mRNA encoding for PTK7 (HUGO Gene Nomenclature Committee (HGNC): 9618), GPC1 (HGNC: 4449), GPC2 (HGNC: 4450), GPC3 (HGNC: 4451), GPC4 (HGNC: 4452), GPC5 (HGNC: 4453), or GPC6 (HGNC: 4454).

The nucleic acid may be a double-stranded siRNA. As the skilled person will appreciate, and as explained further below, a siRNA molecule may also include a short 3′ DNA sequence. Alternatively, the nucleic acid may be a DNA (usually double-stranded DNA) which, when transcribed in a mammalian cell, yields an RNA having two complementary portions joined via a spacer, such that the RNA takes the form of a hairpin when the complementary portions hybridise with each other. In a mammalian cell, the hairpin structure may be cleaved from the molecule by the enzyme DICER, to yield two distinct, but hybridised, RNA molecules.

It is expected that perfect identity/complementarity between the nucleic acid of the invention and the target sequence, although preferred, is not essential. Accordingly, the nucleic acid of the invention may include a single mismatch compared to the mRNA of PTK7, GPC1, GPC2, GPC3, GPC4, GPC5, or GPC6. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides.

The strands that form the double-stranded RNA may have short 3′ dinucleotide overhangs, which may be DNA or RNA. The use of a 3′ DNA overhang has no effect on siRNA activity compared to a 3′ RNA overhang but reduces the cost of chemical synthesis of the nucleic acid strands (Elbashir et al., 2001c). For this reason, DNA dinucleotides may be preferred.

When present, the dinucleotide overhangs may be symmetrical to each other, though this is not essential. Indeed, the 3′ overhang of the sense (upper) strand is irrelevant for RNAi activity, as it does not participate in mRNA recognition and degradation (Elbashir et al., 2001a, 2001b, 2001c).

While RNAi experiments in Drosophila show that antisense 3′ overhangs may participate in mRNA recognition and targeting (Elbashir et al. 2001c), 3′ overhangs do not appear to be necessary for RNAi activity of siRNA in mammalian cells. Incorrect annealing of 3′ overhangs is therefore thought to have little effect in mammalian cells (Elbashir et al. 2001c; Czauderna et al. 2003).

Any dinucleotide overhang may therefore be used in the antisense strand of the siRNA. Nevertheless, the dinucleotide is preferably -UU or -UG (or -TT or -TG if the overhang is DNA), more preferably -UU (or -TT). The -UU (or -TT) dinucleotide overhang is most effective and is consistent with (i.e., capable of forming part of) the RNA polymerase III end of transcription signal (the terminator signal is TTTTT). Accordingly, this dinucleotide is most preferred. The dinucleotides AA, CC and GG may also be used, but are less effective and consequently less preferred. Moreover, the 3′ overhangs may be omitted entirely from the siRNA.

Disclosed herein are single-stranded nucleic acids (herein referred to as single-stranded siRNAs) respectively consisting of a component strand of one of the aforementioned double-stranded nucleic acids, preferably with the 3′-overhangs, but optionally without. Also described are kits containing pairs of such single-stranded nucleic acids, which are capable of hybridising with each other in vitro to form the aforementioned double-stranded siRNAs, which may then be introduced into cells.

Disclosed herein is DNA that, when transcribed in a mammalian cell, yields an RNA (herein also referred to as an shRNA) having two complementary portions which are capable of self-hybridising to produce a double-stranded motif, or a sequence that differs from any one of the aforementioned sequences by a single base pair substitution.

The complementary portions will generally be joined by a spacer, which has suitable length and sequence to allow the two complementary portions to hybridise with each other. The two complementary (i.e., sense and antisense) portions may be joined 5′-3′ in either order. The spacer will typically be a short sequence, of approximately 4-12 nucleotides, preferably 4-9 nucleotides, more preferably 6-9 nucleotides. Preferably the 5′ end of the spacer (immediately 3′ of the upstream complementary portion) consists of the nucleotides -UU- or -UG-, again preferably -UU- (though, again, the use of these particular dinucleotides is not essential). A suitable spacer, recommended for use in the pSuper system of OligoEngine (Seattle, Washington, USA) is UUCAAGAGA. In this and other cases, the ends of the spacer may hybridise with each other by a small number (e.g., 1 or 2) of base pairs. Similarly, the transcribed RNA preferably includes a 3′ overhang from the downstream complementary portion. Again, this is preferably -UU or -UG, more preferably -UU.

Such shRNA molecules may then be cleaved in the mammalian cell by the enzyme DICER to yield a double-stranded siRNA as described above, in which one or each strand of the hybridised dsRNA includes a 3′ overhang.

Techniques for the synthesis of the nucleic acids of the invention are of course well known in the art. The skilled person is well able to construct suitable transcription vectors for the DNA of the invention using well-known techniques and commercially available materials. In particular, the DNA will be associated with control sequences, including a promoter and a transcription termination sequence. Of particular suitability are the commercially available pSuper and pSuperior systems of OligoEngine (Seattle, Washington, USA). These use a polymerase-III promoter (H1) and a T5 transcription terminator sequence that contributes two U residues at the 3′ end of the transcript (which, after DICER processing, provide a 3′ UU overhang of one strand of the siRNA). Another suitable system is described in Shin et al. (RNA, 2009 May; 15 (5): 898-910), which uses another polymerase-III promoter (U6).

The double-stranded siRNAs of the invention may be introduced into mammalian cells in vitro or in vivo using known techniques, as described below, to suppress expression of PTK7, GPC1, GPC2, GPC3, GPC4, GPC5, or GPC6. Similarly, transcription vectors containing the DNAs of the invention may be introduced into tumour cells in vitro or in vivo using known techniques, as described below, for transient or stable expression of RNA, again to suppress expression of PTK7, GPC1, GPC2, GPC3, GPC4, GPC5, or GPC6.

Accordingly, the invention also provides a method of suppressing PTK7, GPC1, GPC2, GPC3, GPC4, GPC5, or GPC6 expression in a mammalian, e.g., human, cell, the method comprising administering to the cell a double-stranded siRNA of the invention or a transcription vector of the invention. Similarly, the invention further provides a method of treating the diseases described herein, the method comprising administering to a subject a double-stranded siRNA of the invention or a transcription vector of the invention.

Materials and methods suitable for the administration of siRNA duplexes and DNA vectors of the invention are well known in the art and improved methods are under development, given the potential of RNAi technology.

Generally, many techniques are available for introducing nucleic acids into mammalian cells. The choice of technique will depend on whether the nucleic acid is transferred into cultured cells in vitro or in vivo in the cells of a patient. Techniques suitable for the transfer of nucleic acid into mammalian cells in vitro include the use of liposomes, electroporation, microinjection, cell fusion, DEAE-dextran and calcium phosphate precipitation. In vivo gene transfer techniques include transfection with viral (typically retroviral) vectors, exosomes or exosome-like particles, naked oligonucleotides (e.g. linked to targeting molecules such as GalNAc (N-acetylgalactosamine) or pegylated oligonucleotides), lipid nanoparticles, and viral coat protein-liposome mediated transfection (Dzau et al. (2003) Trends in Biotechnology 11, 205-210; Roberts T C et al., Nature Rev. Drug Discov., Vol 19, 2020; Paunovska K, Nature Rev. Genetics, Vol 23. 2022).

In particular, suitable techniques for cellular administration of the nucleic acids of the invention both in vitro and in vivo are disclosed in the following articles:

General reviews: Borkhardt, A. 2002. Blocking oncogenes in malignant cells by RNA interference--new hope for a highly specific cancer treatment? Cancer Cell. 2:167-8. Hannon, G. J. 2002. RNA interference. Nature. 418:244-51. McManus, M. T., and P. A. Sharp. 2002. Gene silencing in mammals by small interfering RNAs. Nat Rev Genet. 3:737-47. Scherr. M., M. A. Morgan, and M. Eder. 2003b. Gene silencing mediated by small interfering RNAs in mammalian cells. Curr Med Chem. 10:245-56. Shuey, D. J., D. E. McCallus, and T. Giordano. 2002. RNAi: gene-silencing in therapeutic intervention. Drug Discov Today. 7:1040-6.

Systemic delivery using liposomes: Lewis, D. L., J. E. Hagstrom, A. G. Loomis, J. A. Wolff, and H. Herweijer. 2002. Efficient delivery of siRNA for inhibition of gene expression in postnatal mice. Nat Genet. 32:107-8. Paul, C. P., P. D. Good, I. Winer, and D. R. Engelke. 2002. Effective expression of small interfering RNA in human cells. Nat Biotechnol. 20:505-8. Song, E., S. K. Lee, J. Wang, N. Ince, N. Ouyang, J. Min, J. Chen, P. Shankar, and J. Lieberman. 2003. RNA interference targeting Fas protects mice from fulminant hepatitis. Nat Med. 9:347-51. Sorensen, D. R., M. Leirdal, and M. Sioud. 2003. Gene silencing by systemic delivery of synthetic siRNAs in adult mice. J Mol Biol. 327:761-6.

Virus mediated transfer: Abbas-Terki, T., W. Blanco-Bose, N. Deglon, W. Pralong, and P. Aebischer. 2002. Lentiviral-mediated RNA interference. Hum Gene Ther. 13:2197-201. Barton, G. M., and R. Medzhitov. 2002. Retroviral delivery of small interfering RNA into primary cells. Proc Natl Acad Sci USA. 99:14943-5. Devroe, E., and P. A. Silver. 2002. Retrovirus-delivered siRNA. BMC Biotechnol. 2:15. Lori, F., P. Guallini, L. Galluzzi, and J. Lisziewicz. 2002. Gene therapy approaches to HIV infection. Am J Pharmacogenomics. 2:245-52. Matta, H., B. Hozayev, R. Tomar, P. Chugh, and P. M. Chaudhary. 2003. Use of lentiviral vectors for delivery of small interfering RNA. Cancer Biol Ther. 2:206-10. Qin, X. F., D. S. An, I. S. Chen, and D. Baltimore. 2003. Inhibiting HIV-1 infection in human T cells by lentiviral-mediated delivery of small interfering RNA against CCR5. Proc Natl Acad Sci USA. 100:183-8. Scherr, M., K. Battmer, A. Ganser, and M. Eder. 2003a. Modulation of gene expression by lentiviral-mediated delivery of small interfering RNA. Cell Cycle. 2:251-7. Shen, C., A. K. Buck, X. Liu, M. Winkler, and S. N. Reske. 2003. Gene silencing by adenovirus-delivered siRNA. FEBS Lett. 539:111-4.

Peptide delivery: Morris, M. C., L. Chaloin, F. Heitz, and G. Divita. 2000. Translocating peptides and proteins and their use for gene delivery. Curr Opin Biotechnol. 11:461-6. Simeoni, F., M. C. Morris, F. Heitz, and G. Divita. 2003. Insight into the mechanism of the peptide-based gene delivery system MPG: implications for delivery of siRNA into mammalian cells. Nucleic Acids Res. 31:2717-24. Other technologies that may be suitable for delivery of siRNA to the target cells are based on nanoparticles or nanocapsules such as those described in U.S. Pat. Nos. 6,649,192B and 5,843,509B.

The agent may be an RNA-guided endonuclease system. In some embodiments, the agent is an RNA-guided endonuclease system comprising (a) an RNA-guided endonuclease; and (b) a guide RNA complementary to a target sequence within the target gene (i.e., PTK7, or any one of GPC1, GPC2, GPC3, GPC4, GPC5, or GPC6) in the genome of the target cell.

In some embodiments, the agent is an RNA-guided endonuclease system comprising (a) a nucleic acid sequence encoding an RNA-guided endonuclease; and (b) a nucleic acid sequence encoding a guide RNA complementary to a target sequence within the target gene (i.e., PTK7, or any one of GPC1, GPC2, GPC3, GPC4, GPC5, or GPC6) in the genome of the target cell.

The present invention may also use a CRISPR (“clustered regularly interspaced short palindromic repeats”) system to decrease expression of the target genes. The CRISPR or CRISPR-Cas system is derived from a prokaryotic RNA-guided defence system. There are at least eleven different CRISPR-Cas systems, which have been grouped into three major types (I-III). Type II and type III CRISPR-Cas systems have been adapted as a genome-engineering tool.

Typically, most naturally occurring type II CRISPR-Cas systems employ three components:
a protein endonuclease Cas (CRISPR-associated protein) having DNA nickase activity which is referred to in this specification as an RNA-guided endonuclease (or an RNA-guided DNA endonuclease),
a “targeting” or “guide” RNA (CRISPR-RNA or crRNA) comprising a short sequence, typically of approximately 20nucleotides, complementary to a target sequence (“protospacer”) in the genome, and
a “scaffold” RNA (trans-acting CRISPR RNA or tracrRNA) which interacts with the crRNA and recruits the Cas endonuclease.

In some embodiments, the system introduces a DNA break into the target gene. The target gene may be knocked-out (i.e., made inoperative). In other embodiments of the invention, the endonuclease is non-functional and is used to target the DNA or RNA to reduce expression by means other than introducing DNA cuts and is instead functionalized to inhibit expression of encoded proteins by e.g., inhibition of translation or transcription.

Typically, assembly of these components and hybridisation of the crRNA with its target sequence in the chromosome results in cleavage of the chromosome by the endonuclease, at or close to the target sequence. Cleavage also requires that the target DNA contains a recognition site for the Cas enzyme (protospacer adjacent motif, or PAM) located sufficiently close to the crRNA target sequence, typically immediately adjacent the 3′ end of the target sequence. Cellular repair of the DNA break can lead to the insertion/deletion/mutation of bases and mutation at the target locus.

This three-component system has been simplified by fusing together crRNA and tracrRNA, to create a chimeric single guide RNA (sgRNA or gRNA). Hybridisation of the gRNA with the target sequence leads to cleavage of the target DNA at an adjacent/upstream PAM site. An gRNA can therefore be regarded as comprising a crRNA component (which determines the target sequence) and a tracrRNA component (which recruits the endonuclease).

The protein component of the CRISPR system is referred to as an endonuclease and may have enzymatic activity (i.e., DNA nickase activity) when associated with the appropriate RNA factors. Typically, the endonuclease will cleave chromosomal DNA. In some embodiments, the endonuclease is a Cas9 protein. Examples include Staphylococcus aureus (SaCas9), Streptococcus pyogenes (SpCas9), Neisseria meningitidis (NM Cas9), Streptococcus thermophilus (ST Cas9), Treponema denticola (TD Cas9), or variants thereof. The PAM sequences recognised by these enzymes are well known in the art. Beneficially, SaCas9, CjCas9, and NmCas9 (2.9-3.3 kb) each allow for the packaging of both Cas9 and gRNA in a single AAV vector.

When using a catalytically active endonuclease, the target sequence recognised by the guide RNA may be upstream of a suitable site for insertion.

The endonuclease may comprise a nuclear localisation sequence (NLS) effective in mammalian cells, such as the SV40 large T antigen NLS, which has the sequence PKKKRKV (SEQ ID NO: 30). Other mammalian NLS sequences are known to the skilled person. The endonuclease may comprise multiple copies of an NLS, e.g., two or three copies of an NLS. Where multiple NLS sequences are present, they are typically repeats of the same NLS.

In some embodiments, a gene encoding the endonuclease component of the system will be under transcriptional control of an RNA polymerase II promoter e.g., a viral or human RNA polymerase II promoter. Examples include CMV or SV40 promoter, or a mammalian “housekeeping” promoter. Genes encoding any RNA components (gRNA, crRNA or tracrRNA) will typically be under the transcriptional control of an RNA polymerase III promoter (e.g., a human RNA polymerase Ull promoter) such as the U6 or H1 promoter, or variants thereof which retain or have enhanced activity.

In some embodiments, the agent is a CRISPR-Cas vector system. In some embodiments, the agent is a CRISPR-Cas vector system comprising (a) a Cas endonuclease; and (b) a guide RNA complementary to a target sequence within the target gene (i.e., PTK7, or any one of GPC1, GPC2, GPC3, GPC4, GPC5, or GPC6).

In some embodiments, the agent is a CRISPR-Cas vector system comprising (a) a nucleic acid sequence encoding a Cas endonuclease; and (b) a nucleic acid sequence encoding a guide RNA complementary to a target sequence within the target gene (i.e., PTK7, or any one of GPC1, GPC2, GPC3, GPC4, GPC5, or GPC6).

### Ligand Traps

Ligand traps, also known as decoy receptors, according to the invention comprise an agent comprising a receptor/coreceptor peptide or polypeptide capable of binding to a CCN ligand. In particular, the ligand traps of the invention are capable of binding to any one of CCN1-4 and 6, and fragments thereof (e.g., domains III and IV of CCN1-4 and 6). In some embodiments, the agent specifically binds to a CCN ligand.

The term “peptide” is used herein to refer to short chains of amino acids consisting of 40 or fewer amino acids linked by peptide bonds. The term “polypeptide” is used herein to refer to large biomolecules, or macromolecules, consisting of one or more long chains of amino acid residues, each being more than 40 amino acids in length. In some embodiments, the peptide or polypeptide is isolated. In some embodiments, the peptide or polypeptide is soluble.

Accordingly, an aspect of the invention provides an agent comprising a PTK7 polypeptide or fragment thereof for use in a method of treatment or prophylaxis of fibrosis, metabolic disease, inflammatory disease, autoimmune disease, retinal disease, muscular dystrophy, cardiac disease, or polycystic kidney disease, where said PTK7 polypeptide or fragment thereof binds to a CCN ligand and inhibits binding of said CCN ligand to a transmembrane PTK7 receptor and/or membrane bound GPC. In some embodiments, the PTK7 polypeptide or fragment thereof binds to the CCN ligand (e.g., CCN protein or fragment thereof) and inhibits binding to PTK7 or a GPC in the cell membrane and inhibits CCN ligand-stimulated signalling via PTK7. In some embodiments, the PTK7 polypeptide or fragment thereof is soluble.

In some embodiments, the PTK7 polypeptide or fragment thereof binds to a CCN polypeptide or fragment thereof. For example, the PTK7 polypeptide or fragment thereof binds to CCN1, CCN2, CCN3, CCN4, and/or CCN6. In some embodiments, the PTK7 polypeptide or fragment thereof binds to domains 3&4 of a CCN protein, for example, domains 3&4 of CCN1, CCN2, CCN3, CCN4 and/or CCN6. Such agents inhibit any one of CCN1-4 and 6, and fragments thereof (e.g., domains III and IV of CCN1-4 and 6) binding to PTK7, GPC1, GPC2, GPC3, GPC4, GPC5, or GPC6 in the cell membrane, thereby preventing downstream signalling.

In some embodiments, said PTK7 fragment includes one or more, or all, of the transmembrane domain(s). Accordingly, the PTK7 fragment may comprise or consist of the extracellular domains of PTK7. For example, one or more of the seven immunoglobulin (lg) type loops. In some embodiments, said PTK7 peptide or polypeptide is a recombinant peptide or polypeptide. The polypeptide or fragment may be modified (e.g., modified to increase stability).

In some embodiments, the PTK7 polypeptide comprises or consists of an amino acid sequence having at least 70% identity to SEQ ID NO: 2 (the extracellular portion of PTK7) or SEQ ID NO: 18 (the extracellular portion without a signal peptide). In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 2 or SEQ ID NO: 18. In some embodiments, the PTK7 polypeptide consists of SEQ ID NO: 2 or SEQ ID NO: 18.

In some embodiments, the PTK7 polypeptide comprises or consists of a fragment of SEQ ID NO: 2 or SEQ ID NO: 18. A fragment of PTK7 or the extracellular domain may have a minimum length of 10 amino acids, and a maximum length of one of 15, 20, 25, 30, 40, 50, 100, 110, 120, 130, 140, 150, 200, 250, 300, 350, 400, 450, 500, 550, 600, or 650 amino acids. The fragment may be a peptide or polypeptide. In some embodiments, the fragment comprises an amino acid sequence having at least 70% identity to SEQ ID NO: 2 or SEQ ID NO: 18. In some embodiments, the fragment has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 2 or SEQ ID NO: 18.

In some embodiments, the PTK7 polypeptide comprises or consists of an amino acid sequence having at least 70% identity to SEQ ID NO: 18. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 18. In some embodiments, the PTK7 polypeptide consists of SEQ ID NO: 18. In some embodiments, the PTK7 polypeptide has a maximum length of 675 amino acids.

Another aspect of the invention provides an agent comprising a GPC polypeptide or fragment thereof for use in a method of treatment or prophylaxis of fibrosis, metabolic disease, inflammatory disease, autoimmune disease, cancer, retinal disease, muscular dystrophy, cardiac disease, or polycystic kidney disease, where said GPC or fragment thereof binds a CCN ligand and inhibits binding of said CCN ligand to a membrane bound PTK7 receptor and/or a membrane bound GPC. In some embodiments, the GPC polypeptide is GPC1, GPC2, GPC3, GPC4, GPC5, or GPC6. In preferred embodiments, the GPC polypeptide is GPC1 or GPC4. It is anticipated that the GPC or fragment thereof may bind to a ligand (e.g., a CCN or fragment thereof) and inhibit binding to a GPC in the cell membrane and inhibit CCN ligand-stimulated signalling via PTK7. In some embodiments, the agent prevents the formation of a GPC-CCN ligand-PTK7 complex. In some embodiments, the GPC polypeptide or fragment thereof is soluble.

The GPC polypeptide or fragment may be based on any one of GPC1-6, where said polypeptide or fragment excludes the GPI anchor attachment portion. In some embodiments, a ligand trap based on GPC1, GPC2, GPC3, GPC4, GPC5, or GPC6 may comprise, or consist of, an amino acid sequence corresponding to the sequence excluding the GPI attachment portion. In some embodiments, said GPC polypeptide or fragment is a recombinant polypeptide or fragment. The polypeptide or fragment may be modified (e.g., modified to increase stability).

In some embodiments, the GPC1 polypeptide comprises or consists of an amino acid sequence having at least 70% identity to SEQ ID NO: 3 or SEQ ID NO: 24. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 3 or SEQ ID NO: 24. In some embodiments, the GPC1 polypeptide consists of SEQ ID NO: 3 or SEQ ID NO: 24.

In some embodiments, the GPC1 polypeptide comprises or consists of a fragment of SEQ ID NO: 3 or SEQ ID NO: 24. A fragment of GPC1 may have a minimum length of 10 amino acids, and a maximum length of one of 15, 20, 25, 30, 40, 50, 100, 110, 120, 130, 140, 150, 200, 250, 300, 350, 400, 450, 500, or 550 amino acids. The fragment may be a peptide or polypeptide. In some embodiments, the GPC1 fragment comprises an amino acid sequence having at least 70% identity to SEQ ID NO: 3 or SEQ ID NO: 24. In some embodiments, the fragment has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 3 or SEQ ID NO: 24.

In some embodiments, the GPC1 polypeptide comprises or consists of an amino acid sequence having at least 70% identity to SEQ ID NO: 19. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 19. In some embodiments, the GPC1 polypeptide consists of SEQ ID NO: 19.

SEQ ID NO: 19:
DPASKSRSCGEVRQIYGAKGFSLSDVPQAEISGEHLRICPQGYTCCTS
EMEENLANRSHAELETALRDSSRVLQAMLATQLRSFDDHFQHLLNDSE
RTLQATFPGAFGELYTQNARAFRDLYSELRLYYRGANLHLEETLAEFW
ARLLERLFKQLHPQLLLPDDYLDCLGKQAEALRPFGEAPRELRLRATR
AFVAARSFVQGLGVASDVVRKVAQVPLGPECSRAVMKLVYCAHCLGVP
GARPCPDYCRNVLKGCLANQADLDAEWRNLLDSMVLITDKFWGTSGVE
SVIGSVHTWLAEAINALQDNRDTLTAKVIQGCGNPKVNPQGPGPEEKR
RRGKLAPRERPPSGTLEKLVSEAKAQLRDVQDFWISLPGTLCSEKMAL
STASDDRCWNGMARGRYLPEVMGDGLANQINNPEVEVDITKPDMTIRQ
QIMQLKIMTNRLRSAYNGNDVDFQDASDDGSGSGSGDGCLDDLCSRKV
SRKSSSSRTPLTHALPGLSEQEGQKTS

In some embodiments, the GPC2 polypeptide comprises or consists of an amino acid sequence having at least 70% identity to SEQ ID NO: 4 or SEQ ID NO: 25. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 4 or SEQ ID NO: 25. In some embodiments, the GPC2 polypeptide consists of SEQ ID NO: 4 or SEQ ID NO: 25.

In some embodiments, the GPC2 polypeptide comprises or consists of a fragment of SEQ ID NO: 4 or SEQ ID NO: 25. A fragment of GPC2 may have a minimum length of 10 amino acids, and a maximum length of one of 15, 20, 25, 30, 40, 50, 100, 110, 120, 130, 140, 150, 200, 250, 300, 350, 400, 450, 500, or 550 amino acids. The fragment may be a peptide or polypeptide. In some embodiments, the GPC2 fragment comprises an amino acid sequence having at least 70% identity to SEQ ID NO: 4 or SEQ ID NO: 25. In some embodiments, the fragment has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 4 or SEQ ID NO: 25.

In some embodiments, the GPC3 polypeptide comprises or consists of an amino acid sequence having at least 70% identity to SEQ ID NOs: 5 or 13, or SEQ ID NO: 26. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 5 or 13, or SEQ ID NO: 26. In some embodiments, the GPC3 polypeptide consists of SEQ ID NOs: 5 or 13, or SEQ ID NO: 26.

In some embodiments, the GPC3 polypeptide comprises or consists of a fragment of SEQ ID NOs: 5 or 13, or SEQ ID NO: 26. A fragment of GPC3 may have a minimum length of 10 amino acids, and a maximum length of one of 15, 20, 25, 30, 40, 50, 100, 110, 120, 130, 140, 150, 200, 250, 300, 350, 400, 450, 500, or 550 amino acids. The fragment may be a peptide or polypeptide. In some embodiments, the GPC3 fragment comprises an amino acid sequence having at least 70% identity to SEQ ID NOs: 5 or 13, or SEQ ID NO: 26. In some embodiments, the fragment has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NOs: 5 or 13, or SEQ ID NO: 26.

In some embodiments, the GPC4 polypeptide comprises or consists of an amino acid sequence having at least 70% identity to SEQ ID NO: 6 or SEQ ID NO: 27. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 6 or SEQ ID NO: 27. In some embodiments, the GPC4 polypeptide consists of SEQ ID NO: 6 or SEQ ID NO: 27.

In some embodiments, the GPC4 polypeptide comprises or consists of a fragment of SEQ ID NO: 6 or SEQ ID NO: 27. A fragment of GPC4 may have a minimum length of 10 amino acids, and a maximum length of one of 15, 20, 25, 30, 40, 50, 100, 110, 120, 130, 140, 150, 200, 250, 300, 350, 400, 450, 500, or 550 amino acids. The fragment may be a peptide or polypeptide. In some embodiments, the GPC4 fragment comprises an amino acid sequence having at least 70% identity to SEQ ID NO: 6 or SEQ ID NO: 27. In some embodiments, the fragment has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 6 or SEQ ID NO: 27.

In some embodiments, the GPC5 polypeptide comprises or consist of an amino acid sequence having at least 70% identity to SEQ ID NO: 7 or SEQ ID NO: 28. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 7 or SEQ ID NO: 28. In some embodiments, the GPC5 polypeptide consists of SEQ ID NO: 7 or SEQ ID NO: 28.

In some embodiments, the GPC5 polypeptide comprises or consists of a fragment of SEQ ID NO: 7 or SEQ ID NO: 28. A fragment of GPC5 may have a minimum length of 10 amino acids, and a maximum length of one of 15, 20, 25, 30, 40, 50, 100, 110, 120, 130, 140, 150, 200, 250, 300, 350, 400, 450, 500, or 550 amino acids. The fragment may be a peptide or polypeptide. In some embodiments, the GPC5 fragment comprises an amino acid sequence having at least 70% identity to SEQ ID NO: 7 or SEQ ID NO: 28. In some embodiments, the fragment has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 7 or SEQ ID NO: 28.

In some embodiments, the GPC6 polypeptide comprises or consists of an amino acid sequence having at least 70% identity to SEQ ID NO: 8 or SEQ ID NO: 29. In some embodiments, the polypeptide has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 8 or SEQ ID NO: 29. In some embodiments, the GPC6 polypeptide consists of SEQ ID NO: 8 or SEQ ID NO: 29.

In some embodiments, the GPC6 polypeptide comprises or consists of a fragment of SEQ ID NO: 8 or SEQ ID NO: 29. A fragment of GPC6 may have a minimum length of 10 amino acids, and a maximum length of one of 15, 20, 25, 30, 40, 50, 100, 110, 120, 130, 140, 150, 200, 250, 300, 350, 400, 450, 500, or 550 amino acids. The fragment may be a peptide or polypeptide. In some embodiments, the GPC6 fragment comprises an amino acid sequence having at least 70% identity to SEQ ID NO: 8 or SEQ ID NO: 29. In some embodiments, the fragment has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 8 or SEQ ID NO: 29.

Percent (%) amino acid sequence identity with respect to a reference sequence is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Various known tools can be used to measure sequence identity, including but not limited to Clustal Omega, Multiple Sequence Alignment (EMBL-EBI). In some embodiments, the % sequence identity is over the length of the polypeptide or the fragment.

In some embodiments, a sequence at least 75%, at least 80%, at least 85%, at least 90%, at least 95%, at least 96%, at least 97%, at least 98%, at least 99%, or 100% sequence identity to an amino acid sequence described herein.

In some embodiments, the peptide or polypeptide comprises an amino acid sequence having at least 70% sequence identity to the mature sequence of PTK7, GPC1, GPC2, GPC3, GPC4, GPC5, or GPC6.

In some embodiments, a ligand trap may be able to bind any one of CCN1-4 and 6, and fragments thereof (e.g., domains III and IV of CCN1-4 and 6), e.g., with binding affinity of at least 100 μM or less, optionally one of 10 μM or less, 1 μM or less, 100 nM or less, or about 1 to 100 nM.

The agent may comprise a heterologous moiety. In some embodiments, the heterologous moiety increases the stability of the agent. In some embodiments, the heterologous moiety increases the serum half-life of the agent. The heterologous moiety may be defined as a polypeptide or peptide which is not present in combination (i.e., not linked to, directly or indirectly) with said PTK7 or GPC polypeptide or peptide in nature. In some embodiments, the peptide heterologous moiety is a non-PTK7 or non-GPC peptide sequence.

In some embodiments, the heterologous moiety is a chain modification. In some embodiments, the heterologous moiety is a biomolecule. The biomolecule may be a polypeptide or peptide sequence. In some embodiments, the heterologous moiety is a recombinant polypeptide or peptide sequence. In some embodiments, where the heterologous moiety is a peptide, said peptide is a fusion protein with said PTK7 polypeptide or fragment thereof. In some embodiments, where the heterologous moiety is a peptide, said peptide is a fusion protein with said GPC polypeptide or fragment thereof. In other words, the agent is typically a recombinant sequence comprising a heterologous moiety polypeptide or peptide fused to the PTK7 or GPC polypeptide or peptide. The heterologous moiety may be N-terminally fused. Alternatively, the heterologous moiety may be C-terminally fused. In some embodiments, the peptide or polypeptide is covalently attached to the PTK7 or GPC polypeptide or peptide. In some embodiments, the peptide or polypeptide is non-covalently attached to the PTK7 or GPC polypeptide or peptide.

In some embodiments, the heterologous moiety is at least 20, 50, 100, 200, 300, 400 or 500 amino acids long. In some embodiments, the heterologous moiety is selected from the group consisting of an Fc fragment, serum albumin (e.g., human serum albumin (HSA)), fibrinogen, glutathione S-transferase, transferrin, or streptavidin. In some embodiments, the heterologous moiety is a monomeric Fc. In some embodiments, the heterologous moiety is dimeric Fc. The Fc fragment may be an Fc fragment of any subclass or chimera of any subclasses. In some embodiments, the Fc fragment is a IgG1, IgG2, or IgG4 Fc fragment. IgG1, IgG2 and IgG4 are often preferred to IgG3 due to their longer half-lives.

In some embodiments, the heterologous moiety comprises an amino acid sequence having 70% sequence identity to SEQ ID NO: 20. In some embodiments, the heterologous moiety is an amino acid sequence comprising SEQ ID NO: 20.

SEQ ID NO: 20 (Fc domain):
IEGRMDPKSCDKTHTCPPCPAPELLGGPSVFLFPPKPKDTLMISRTPE
VTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPS
RDELTKNQVSLTCLVKGFYPSDIAVEWESNGQPENNYKTTPPVLDSDG
SFFLYSKLTVDKSRWQQGNVFSCSVMHEALHNHYTQKSLSLSPGK

In some embodiments, the heterologous moiety is a carbohydrate molecule. Examples, include dextran, glycosylation, polysialylation, hydroxyethylation (HESylation), heparosanylation (HEPylation), and hyaluronic acid polysaccharide (HAylation).

In some embodiments, the heterologous moiety is a polymer. Polymers may include synthetic polymers or polypeptides. For example, synthetic polymer may include PEG. In some embodiments, the heterologous moiety is a chemical modification. The chemical modification may include PEGylation. Any suitable chemical modification known in the art for use in increasing stability of therapeutic proteins may be used. Example polypeptides polymers include XTEN (protein polymer developed by Amunix), PASylation (proline-alanine-serine polymer), ELPylation (elastin-like polypeptides), HAPylation (repeated sequence of a glycine rich—Gly4Ser)ₙ polypeptide, gelatin-like protein (GLK—has a (Gly-XY)ₙ structure).

In some embodiments, the heterologous moiety is a lipid. Lipidation involves the transfer of a lipid group to a protein and can be used to increase the half-life of therapeutic proteins, reduce immunogenicity, and increase cell membrane permeability (Menacho-Melgar, 2019., J Control Release (295) 1-12). Without wishing to be bound by theory, it is believed that lipidation improves drug half-life by enabling the binding of albumin that is present in the blood of a subject. Example lipid modifications include, luarate, myristate, and palmitate.

Any heterologous moiety known in the art for use with therapeutic proteins may be used.

The heterologous moiety as described herein may be linked directly or indirectly via a linker. Any linker may be used. In some embodiments, the linker is a peptide linker. The linker may be at least 5, 10, 20, 50, or 100 amino acids in length. The linker does not include a sequence from the peptide heterologous moiety, PTK7, or a GPC.

In some embodiments, the agent comprising a PTK7 polypeptide comprises an amino acid sequence having at least 70% sequence identity to SEQ ID NO: 21 or a fragment thereof. In some embodiments, the agent has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 21. In some embodiments, the agent consists of SEQ ID NO: 21.

SEQ ID NO 21:
AIVFIKQPSSQDALQGRRALLRCEVEAPGPVHVYWLLDGAPVQDTERR
FAQGSSLSFAAVDRLQDSGTFQCVARDDVTGEEARSANASFNIKWIEA
GPVVLKHPASEAEIQPQTQVTLRCHIDGHPRPTYQWFRDGTPLSDGQS
NHTVSSKERNLTLRPAGPEHSGLYSCCAHSAFGQACSSQNFTLSIADE
SFARVVLAPQDVVVARYEEAMFHCOFSAQPPPSLQWLFEDETPITNRS
RPPHLRRATVFANGSLLLTQVRPRNAGIYRCIGQGQRGPPIILEATLH
LAEIEDMPLFEPRVFTAGSEERVTCLPPKGLPEPSVWWEHAGVRLPTH
GRVYQKGHELVLANIAESDAGVYTCHAANLAGQRRQDVNITVATVPSW
LKKPQDSQLEEGKPGYLDCLTQATPKPTVVWYRNQMLISEDSRFEVFK
NGTLRINSVEVYDGTWYRCMSSTPAGSIEAQARVQVLEKLKFTPPPQP
QQCMEFDKEATVPCSATGREKPTIKWERADGSSLPEWVTDNAGTLHFA
RVTRDDAGNYTCIASNGPQGQIRAHVQLTVAVFITFKVEPERTTVYQG
HTALLQCEAQGDPKPLIQWKGKDRILDPTKLGPRMHIFQNGSLVIHDV
APEDSGRYTCIAGNSCNIKHTEAPLYVVDKPVPEESEGPGSPPPYKMI
QTIEGRMDPKSCDKTHTCPPCPAPELLGGPSVFLFPPKPKDTLMISRT
PEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLP
PSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

In some embodiments, the agent comprising a GPC-1 polypeptide comprises an amino acid sequence having at least 70% sequence identity to SEQ ID NO: 22 or a fragment thereof. In some embodiments, the agent has an amino acid sequence having at least 75%, at least 80%, at least 85%, at least 90%, at least 95%, at least 96%, at least 97%, at least 98%, at least 99%, or 100% sequence identity to the amino acid sequence of SEQ ID NO: 22.

SEQ ID NO: 22:
DPASKSRSCGEVRQIYGAKGFSLSDVPQAEISGEHLRICPQGYTCCTS
EMEENLANRSHAELETALRDSSRVLQAMLATQLRSFDDHFQHLLNDSE
RTLQATFPGAFGELYTQNARAFRDLYSELRLYYRGANLHLEETLAEFW
ARLLERLFKQLHPQLLLPDDYLDCLGKQAEALRPFGEAPRELRLRATR
AFVAARSFVQGLGVASDVVRKVAQVPLGPECSRAVMKLVYCAHCLGVP
GARPCPDYCRNVLKGCLANQADLDAEWRNLLDSMVLITDKFWGTSGVE
SVIGSVHTWLAEAINALQDNRDTLTAKVIQGCGNPKVNPQGPGPEEKR
RRGKLAPRERPPSGTLEKLVSEAKAQLRDVQDFWISLPGTLCSEKMAL
STASDDRCWNGMARGRYLPEVMGDGLANQINNPEVEVDITKPDMTIRQ
QIMQLKIMTNRLRSAYNGNDVDFQDASDDGSGSGSGDGCLDDLCSRKV
SRKSSSSRTPLTHALPGLSEQEGQKTSHHHHHH

### Medical Use

An aspect of the invention provides the agents described herein for use in therapy. Also provided are the agents described herein for use in a method of treatment or prophylaxis of fibrosis, metabolic disease, inflammatory disease, autoimmune disease, cancer, retinal disease, muscular dystrophy, cardiac disease, or polycystic kidney disease.

The invention further provides a method of treatment or prophylaxis of fibrosis, metabolic disease, inflammatory disease, autoimmune disease, cancer, retinal disease, muscular dystrophy, cardiac disease, or polycystic kidney disease comprising administering one or more of the agents as described herein to a subject. In a further aspect, the present invention provides the use of an agent as described herein for the manufacture of a medicament for the treatment or prophylaxis of fibrosis, metabolic disease inflammatory disease, autoimmune disease, cancer, retinal disease, muscular dystrophy, cardiac disease, or polycystic kidney disease in a subject.

With the knowledge of the key role of CCN proteins in the pathogenesis of diseases characterised by fibrosis, agents as described herein that reduce CCN signalling via PTK7 elicited by the four domain CCN proteins (i.e., CCN1, CCN2, CCN3, CCN4 and/or CCN6) are expected to be efficacious for the treatment of such fibrotic diseases. The exception being CCN5 whose activity is anti-fibrotic and is described in the art as being an endogenous inhibitor of the four-domain CCN protein, CCN2 (Yoon P O, et al. J Mol Cell Cardiol 2010). Therefore, agents described herein that reduce four-domain CCN signalling via PTK7 are expected to be efficacious for treatment of diseases for which CCN5 activity has been suggested to be beneficial.

The data supporting a role for the four domain CCN proteins in disease is vast. The general preclinical models do not differentiate on the underlying aetiology, thus data that show e.g. a pro-fibrotic activity of a four-domain CCN protein (CCN1, CCN2, CCN3, CCN4 and/or CCN6) in a preclinical model of lung fibrosis can usually not be taken as evidence of a pro-fibrotic effect of CCN protein in question to a particular human lung disease, but is rather to be taken as a demonstration the pro-fibrotic activity of the CCN protein question for fibrotic lung diseases in general. Therefore, agents that reduce four domain CCN signalling via PTK7 (PTK7 signalling) is expected to be useful for treating fibrotic diseases. The disclosed agents may be useful for treating fibrotic diseases in at least lungs, eyes, heart, skeletal muscle, peritoneum, kidney, liver, biliary tract, skin, and blood vessels.

Accordingly, a preferred aspect of the invention provides agents as described herein for use in a method of treatment or prophylaxis of fibrosis. In an embodiment of the invention, the agent is for use in a method of treatment or prophylaxis of fibrosis of the lung (pulmonary fibrosis). fibrosis of the eyes, fibrosis of the heart, fibrosis of the skeletal muscle, fibrosis of the peritoneum, fibrosis of the kidney, fibrosis of the liver, fibrosis of the biliary tract, fibrosis of the skin, fibrosis of blood vessels, system sclerosis, bone marrow, surrounding implants or epidural fibrosis.

Pulmonary fibrosis of several aetiologies, including idiopathic pulmonary fibrosis (IPF), systemic sclerosis and other chronic inflammatory diseases affecting the lungs, occupational lung diseases (e.g., toxic exposures, for which the pre-clinical model is the same as the one used for IPF), asthma, or bronchopulmonary dysplasia may be treated using the agents described herein (Richeldi L, et al. Lancet Respir Med 2020: Sternlicht M D, et al. Respir Res 2018; Wang Q, et al. Fibrogenesis Tissue Repair 2011; Wang X, et al. Respirology 2011; Gao W, et al. PLOS One 2014; Zhang L, et al. Int J Mol Med 2014; Alapati D, et al. Am J Respir Cell Mol Biol).

In the context of the invention, fibrosis of the eye may include retinal fibrosis, such as, diabetic retinopathy, age-related macular degeneration, or retinal detachment (Daftarian N, et al. Exp Eye Res 2019; Bagheri A, et al. Mol Vis 2015; Hu B, et al. Int J Mol Sci 2014; Yoon A, et al. PLOS One 2018). Fibrosis of the eye may also include oxygen-induced retinopathy (You J J, et al., Invest Ophthalmol Vis Sci 2009) and glaucoma (Wallace D M, et al. Invest Ophthalmol Vis Sci 2013).

Fibrosis of the heart may include hypertrophy, cardiac fibrosis, heart failure (Yoon P O, et al. J Mol Cell Cardiol 2010; Dorn et al., J Mol Cell Cardiol, 121, 205-211, August 2018; Jeong D, et al. J Am Coll Cardiol 2016; Bickelhaupt S, et al. J Natl Cancer Inst 2017), post-transplant graft fibrosis (Booth A J, et al., Am. J. Transplant. 10 (2010) 220-230), or cardiomyopathy associated fibrosis and reduced cardiac function (Chatzifrangkeskou M, et al. Hum Mol Genet 2016; Koshman Y E, et al. J Mol Cell Cardiol 2015).

Fibrosis of the skeletal muscle may include Duchenne muscular dystrophy (Morales M G, et al. Hum Mol Genet 2013), inactivity (denervation)-induced fibrosis (Rebolledo D L, et al. Matrix Biol 2019) or overuse-induced fibrosis (Barbe M F. et al. J Orthop Res 2019).

Fibrosis of the peritoneum may include peritoneal fibrosis (Sakai N, et al. Sci Rep 2017).

Fibrosis of the kidney may include diabetic nephropathy (Adler S G, et al. Clin J Am Soc Nephrol 2010; Dai H, et al. Ren Fail 2016; Guha et al. FASEB J. 2007 October; 21 (12): 3355-68; Yokoi H et al., Kidney Int. 2008 February; 73 (4): 446-55), chronic kidney disease (Wang Q, et al. Fibrogenesis Tissue Repair 2011; Lai C F, et al. PLOS One 2013; Qian H S, et al. PLOS One 2016; Johnson B G, et al. J Am Soc Nephrol 2017; Kinashi H, et al. Kidney Int 2017), acute kidney injury (Lai C F, et al. Am J Physiol Renal Physiol 2014., Kinashi H, et al. Kidney Int 2017), tubulointerstitial fibrosis (Okada H, et al., J. Am. Soc. Nephrol. 16 (2005) 133-143), chronic allograft nephropathy (associated with CCN2 expression) (Cheng O, et al., Am. J. Transplant.6 (2006) 2292-2306), and glomerulopathies (Toda et al. (Sci Rep. 2017 Feb. 13; 7:42114), e.g., such as focal segmental glomerulosclerosis or Alport syndrome (Ito et al. Kidney International, Vol. 53 (1998), pp. 853-861).

Fibrosis of the liver may include several aetiologies including non-alcoholic steatohepatitis and fatty liver disease (NASH) (Wang Q, et al. Fibrogenesis Tissue Repair 2011; Li S, et al. Sci Rep 2016; Uchio K, et al., Wound Repair Regen. 12 (2004) 60-66; Zhang C Y, et al. Chinese Journal of Applied Physiology, 29 (5): 411-415, 2013).

Fibrosis of the pancreas may include chronic pancreatitis (di Mola F F, et al. Ann Surg 1999).

Fibrosis of biliary tract may include biliary fibrosis (Pi L, et al. Hepatology 2015).

Fibrosis of the skin may include keloids or a scar of any cause (e.g., post-surgical) (Jensen J, et al. Plast Reconstr Surg 2018).

Fibrosis of the blood vessels may include atherosclerosis (Yao Y, et al. Nanomedicine 2017).

It is also anticipated that the agents of the invention may be used in a method of treatment or prophylaxis of systemic sclerosis (Makino K, et al. Arthritis Res Ther 2017) or epidural fibrosis (Xu H, et al. Int J Mol Med 2015).

The terms “treatment”, “treat”, or “treating” are used herein to refer to the reduction in severity of a disease or condition, the reduction in the duration of a disease; the amelioration or elimination of one or more symptoms associated with a disease or condition, or the provision of beneficial effect to a subject with a disease or condition. The term also encompasses prophylaxis of a disease or condition or its symptoms thereof. “Prophylaxis” is known in the art to mean decreasing or reducing the occurrence or severity of a particular disease outcome. For example, delaying progression of cancer in a subject.

In some embodiments, the agent of the invention is administered to a subject. Depending on the agents described herein may be administered in a suitable format/route, including, but not limited to oral, by inhalation, intravenous, subcutaneous, intradermal, intraperitoneal, intrapleural, intraocular, intraarticular, intrathecal, intratumorally, locally into organ the target organ(s) or administered as part of a medical device, e.g., medical or aesthetic implants.

Administration is preferably in a “prophylactically effective amount” or a “therapeutically effective amount”, this being sufficient to show benefit to the individual. The actual amount administered, and rate and time-course of administration, may depend on the individual subject and the nature and severity of their condition.

As used herein, the term “subject” refers to a human or any non-human animal (e g, mouse, rat, rabbit, dog, cat, cattle, swine, sheep, horse or primate). In many embodiments, a subject is a human being. A subject can be a patient, which refers to a human presenting to a medical provider for diagnosis or treatment of a disease. The term “subject” is used herein interchangeably with “individual” or “patient.” In some embodiments, the subject is human. A subject can be afflicted with or is susceptible to a disease or disorder but may or may not display symptoms of the disease or disorder. In some embodiments, the subject is affected or is likely to be affected with fibrosis, cancer, a metabolic disease, or an inflammatory disease, autoimmune disease, retinal disease, muscular dystrophy, cardiac disease, or polycystic kidney disease.

The CCN proteins have also been shown the play a role in metabolic diseases, e.g., such as diabetes and other conditions characterised by insulin resistance (Kim J, et al. PLOS One 2018). Accordingly, it is predicted that agents targeting of signalling elicited by four-domain CCN proteins via PTK7 (PTK7 signalling), as described herein, will be useful for the treatment of this group of diseases.

Accordingly, an aspect of the invention provides an agent as described herein for use in a method of treatment or prophylaxis of a metabolic disease. In some embodiments, the metabolic disease is diabetes.

CCN proteins have also been shown to affect the pathogenesis of inflammatory and/or autoimmune diseases, such as rheumatoid arthritis (Wei J L, et al. Arthritis Rheumatol 2018., Nozawa K, et al. Arthritis Rheum 2013, Amyotrophic Lateral Sclerosis: Gonzalez D, et al. Hum Mol Genet 2018) and inflammatory bowel disease (including ulcerative colitis and Crohn's disease) (Song Z M, et al. Biomed Pharmacother 2019). Therefore, agents described herein that reduce four domain CCN protein signalling via PTK7 (PTK7 signalling) are expected to be efficacious for the treatment of these diseases.

Accordingly, an aspect of the invention provides an agent as described herein for use in a method of treatment or prophylaxis of inflammatory and/or autoimmune diseases. In some embodiments, the inflammatory and/or autoimmune diseases is rheumatoid arthritis or inflammatory bowel disease (IBD). In some embodiments, the IBD is ulcerative colitis (UC) or Crohn's disease (CD).

CCN proteins are also known to affect the development of cancerous diseases in multiple ways, including by contributing to tumour growth, metastasis, chemoresistance and immunotherapy resistance by acting on the cancer cells directly or the tumour stroma. Accordingly, it is predicted that agents targeting of signalling elicited by four-domain CCN proteins via PTK7 (PTK7 signalling) as described herein will be useful for the treatment of cancer. In particular, CCN proteins have been known to affect the development of the following cancerous diseases: cancer of the pancreas (e.g., pancreatic ductal adenocarcinoma (PDA) (Aikawa T, et al. Mol Cancer Ther 2006, PDA, Dhar G, et al., Cancer Lett. 254:63-70, 2007; PDA chemoresistance: Neesse A, et al. Proc Natl Acad Sci USA 2013, Maity G, et al. Mol Cancer Ther 2019 PDA metastasis: Dornhofer N, et al. Cancer Res 2006)); breast cancer (chemoresistance: Wang M Y, et al. Cancer Res 2009; tumour growth and metastasis: Lin J, et al. Cancer Immunol Immunother 2012. Banerjee S, et al. Neoplasia. 5:63-73, 2003, Haque I, et al. Oncogene (2015) 34, 3152-3163, Sarkar S, et al. Oncogenesis 2017; metastasis: Shimo T, et al. J Bone Miner Res 2006, Kang Y, et al. Cancer Cell 2003; immune-evasion: Akalay I, et al. Oncogene 2015); prostate cancer (metastasis: Ono M, et al. PLOS One 2013); cervical cancer (chemoresistance: Rho S B, et al. Biotechnol Lett 2009); ovarian carcinoma (Gery, S. et al. Clin. Cancer Res. 11, 7243-7254 (2005)); liver cancer (e.g., hepatocellular carcinoma (Makino Y, et al. Cancer Res 2018)); bladder cancer (e.g., urothelial bladder cancer (chemoresistance: Wang X, et al. Oncotarget 2017); brain cancer (e.g., glioblastoma (chemoresistance: Zeng H, et al. Cell Death Dis 2017)); bone cancer (e.g., osteosarcoma (chemoresistance: Tsai H C, et al. J Cell Physiol 2019., Tsai H C, et al. PLOS One 2014)); blood cancer (e.g. Acute lymphoblastic leukemia (chemoresistance): Lu H, et al. Ann Hematol 2014) melanoma (metastasis and tumour growth: Finger E C, et al. Oncogene 2014); lung cancer (e.g., mesothelioma (Ohara Y, et al. Oncotarget 2018)); stomach cancer (e.g., gastric carcinoma (Tanaka S. et al. Oncogene 2001, Ji J. et al., Br J Cancer. 2015; 113 (6): 921-933)); mouth cancer (e.g., oral squamous cell carcinoma (Sun S, et al. Cell Biol Int 2020)); oesophageal cancer (Chai D M, et al. J Exp Clin Cancer Res 2019); colorectal cancer (Frewer K A, et al. Cancer Genomics Proteomics 2013); and lung cancer (WO2020188081).

An aspect of the invention therefore provides an agent as described herein for use in a method of treatment or prophylaxis of cancer. In some embodiments, the cancer is selected from the group consisting of pancreatic cancer, breast cancer, prostate cancer, cervical cancer, ovarian cancer, liver cancer, bladder cancer, brain cancer, bone cancer, blood cancer, melanoma, stomach cancer, mouth cancer, oesophageal cancer, colorectal cancer, or lung cancer. In some embodiments, the pancreatic cancer is pancreatic ductal adenocarcinoma. In some embodiments, the liver cancer is hepatocellular carcinoma. In some embodiments, the bladder cancer is urothelial bladder cancer. In some embodiments, the brain cancer is glioblastoma. In some embodiments, the bone cancer is osteosarcoma. In some embodiments the blood cancer is acute lymphoblastic leukemia. In some embodiments, the lung cancer is mesothelioma. In some embodiments, the stomach cancer is gastric carcinoma. In some embodiments, the mouth cancer is oral squamous cell carcinoma.

Furthermore, CCN proteins have been implicated in diseases through mechanisms beyond its pro-fibrotic such as cyst growth in polycystic kidney disease, heart failure and cardiomyocyte hypertrophy in cardiac disease (Jeong D et al, 2016 and Vainio et al. 2019), insulin resistance and neovascular age related macular degeneration. Therefore, in an embodiment of the invention the agent is for use in the treatment of these diseases. The CCN proteins are therefore shown to be drivers of retinal disease (such as diabetic retinopathy or age-related macular degeneration) (N. Daftarian et al. Experimental Eye Research 208 (2021) 108622; N. Daftarian, et al., Experimental Eye Research 184 (2019) 286-295, Hu B et al., Int. J. Mol. Sci. 2014, 15), muscle dystrophies (such as Duchenne Muscular Dystrophy) (Morales M G et al., Human Molecular Genetics, 2013, Vol. 22, No. 24, Petrosino J M et al., FASEB J, VOL 33, February 2019) or polycystic kidney disease (both in terms of fibrosis and cyst growth, see Dwivedi N, et al., 2020, 31 (8): 1697-1710).

### Pharmaceutical Compositions

The agents described herein can be formulated in pharmaceutical compositions.

The invention provides a composition comprising (i) the agent that binds to PTK7 and has PTK7 antagonist activity as described herein, (ii) the agent that inhibits expression of PTK7 as described herein, (iii) the agent comprising a PTK7 polypeptide or fragment thereof as described herein, (iv) the agent that binds to a GPC and inhibits GPC-CCN ligand-PTK7 complex formation as described herein, (v) the agent that inhibits expression of a GPC as described herein, or (vi) the agent comprising a GPC polypeptide or fragment thereof as described herein, for use in therapy.

Another aspect of the invention provides composition comprising (i) the agent that binds to PTK7 and has PTK7 antagonist activity as described herein, (ii) the agent that inhibits expression of PTK7 as described herein, (iii) the agent comprising a PTK7 polypeptide or fragment thereof as described herein, (iv) the agent that binds to a GPC and inhibits GPC-CCN ligand-PTK7 complex formation as described herein, (v) the agent that inhibits expression of a GPC as described herein, or (vi) the agent comprising a GPC polypeptide or fragment thereof as described herein, for use in a method of treatment or prophylaxis of fibrosis, metabolic disease, inflammatory disease, autoimmune disease, cancer, retinal disease, muscular dystrophy, cardiac disease, or polycystic kidney disease.

An aspect of the invention provides a composition comprising two more agents selected from the group consisting of (i) the agent that binds to PTK7 and has PTK7 antagonist activity as described herein, (ii) the agent that inhibits expression of PTK7 as described herein, (iii) the agents comprising a PTK7 polypeptide or fragment thereof as described herein, (iv) the agent that binds to a GPC and inhibits GPC-CCN ligand-PTK7 complex formation as described herein. (v) the agent that inhibits expression of a GPC as described herein, or (vi) the agent comprising a GPC polypeptide or fragment thereof as described herein, for use in therapy.

Another aspect of the invention provides a composition comprising two more agents selected from the group consisting of (i) the agent that binds to PTK7 and has PTK7 antagonist activity as described herein, (ii) the agent that inhibits expression of PTK7 as described herein, (iii) the agent comprising a PTK7 polypeptide or fragment thereof as described herein, (iv) the agent that binds to a GPC and inhibits GPC-CCN ligand-PTK7 complex formation as described herein, (v) the agent that inhibits expression of a GPC as described herein, or (vi) the agent comprising a GPC polypeptide or fragment thereof as described herein, for use in a method of treatment or prophylaxis of fibrosis, metabolic disease, inflammatory disease, autoimmune disease, cancer, retinal disease, muscular dystrophy, cardiac disease, or polycystic kidney disease.

Pharmaceutical compositions may comprise, in additional to one of the above substances, a pharmaceutically acceptable excipient, carrier, buffer, stabiliser or other material well known to those skilled in the art. Such substances should be non-toxic and should not interfere with the efficacy of the active ingredient. In some embodiments, the composition further comprises a pharmaceutically acceptable excipient.

The nucleic acid-containing compositions of the invention can be stored and administered in a sterile physiologically acceptable carrier, where the nucleic acid is dispersed in conjunction with any agents which aid in the introduction of the nucleic acids into cells.

Various sterile solutions may be used for administration of the composition, including water, PBS, ethanol, lipids, etc. The concentration of the agent will be sufficient to provide a therapeutic dose, which will depend on the efficiency of transport into the cells.

Pharmaceutical compositions may be prepared using a pharmaceutically acceptable “carrier” composed of materials that are considered safe and effective. The term “carrier” refers to diluents, binders, lubricants and disintegrants. Those with skill in the art are familiar with such pharmaceutical carriers and methods of compounding pharmaceutical compositions using such carriers. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204 (s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

The composition described herein may be administered alone or in combination with other treatments, either simultaneously or sequentially dependent upon the condition to be treated.

In some diseases (for example some of the diseases described herein), several pathogenic processes co-occur. Therefore, there is a need for combination therapies comprising agents that target multiple pathogenic processes at once (i.e., targets pathogenic processes other than those affected by signalling included by four domain CCN proteins, CCN1, CCN2, CCN3, CCN4 and/or CCN6). Additional therapeutic agents may include anti-diabetic agents (e.g., GLP-1 receptor agonists, SGLT2-inhibitors, thiazolidinediones, DPP4-inhibitors, biguanides, sulfonylureas, GLP-1/GIP1-receptor agonists, etc.), anti-obesity agents (e.g., GLP1-receptor agonists, GLP-1/GIP-receptor agonists, etc.), anti-atherogenic agents (e.g., statins, etc.), retinopathy treatments (e.g., anti-angiogenic interventions such as aflibercept, Brolucizumab, Ranibizumab, Faricimab, etc.), immunotherapeutic agents (e.g., check-point inhibitors such as PD1/PD-L1 inhibitors, CTLA4-inhibitor), cell-therapies directed towards other molecular targets than PTK7 or cancer vaccines directed towards other molecular target than PTK7.

Accordingly, provided is a composition comprising (a) (i) an agent that binds to PTK7 and has PTK7 antagonist activity as described herein, (ii) an agent that inhibits expression of PTK7 as described herein, (iii) an agent comprising a PTK7 polypeptide or fragment thereof as described herein. (iv) an agent that binds to a GPC and inhibits GPC-CCN ligand-PTK7 complex formation as described herein, (v) an agent that inhibits expression of a GPC as described herein, and/or (vi) an agent comprising a GPC polypeptide or fragment thereof as described herein; and (b) at least one other therapeutic agent, for use in therapy.

Also provided is a composition comprising (a) (i) an agent that binds to PTK7 and has PTK7 antagonist activity as described herein, (ii) an agent that inhibits expression of PTK7 as described herein, (iii) an agent comprising a PTK7 polypeptide or fragment thereof as described herein, (iv) an agent that binds to a GPC and inhibits GPC-CCN ligand-PTK7 complex formation as described herein, (v) an agent that inhibits expression of a GPC as described herein, and/or (vi) an agent comprising a GPC polypeptide or fragment thereof as described herein and (b) at least one other therapeutic agent, for use in a method of treatment or prophylaxis of fibrosis, metabolic disease, or inflammatory disease, autoimmune disease, cancer, retinal disease, muscular dystrophy, cardiac disease, or polycystic kidney disease.

In some embodiments, the at least one other therapeutic agent is selected from the group consisting of an anti-diabetic agent, an anti-obesity agent, an anti-atherogenic agent, a retinopathy treatment, an immunotherapeutic agent, a cell therapy directed towards a target other than PTK7, GPC1, GPC2, GPC3, GPC4, GPC5, or GPC6, and a cancer vaccine directed to a target other than PTK7, GPC1, GPC2, GPC3, GPC4, GPC5, or GPC6.

### Methods of Screening

An aspect of the invention provides a method of screening for an agent that binds to PTK7 and has PTK7 antagonist activity, where the agent inhibits CCN ligand-induced signalling. The method comprising:
(i) providing a PTK7 or a fragment thereof;
(ii) contacting said PTK7 with one or more candidate agents:
(iii) determining whether the one or more candidate agents bind to said PTK7 or fragment thereof;
(iv) selecting a candidate agent that binds to said PTK7 or fragment thereof from said one or more candidate agents;
(v) providing a cell expressing PTK7;
(vi) contacting said cell with the candidate agent that binds to a PTK7 or a fragment thereof; and
(vii) determining whether the candidate agent inhibits CCN ligand-induced signalling.

The method may be carried out in the presence of a control agent. For example, step (ii) may also comprise contacting said PTK7 or fragment thereof with a control agent. Step (vi) may also comprise contacting a cell expressing PTK7 with a control agent. When the method further comprises a control agent, the method may also comprise providing more than one PTK7 or fragment thereof and/or providing more than one cell expressing PTK7.

Candidate agent binding to PTK7 may be determined by SPR or thermal shift assay. Without wishing to be bound by theory, a candidate agent that binds to PTK7 or a fragment thereof and has PTK7 antagonist activity, and which inhibits CCN ligand-induced signalling via PTK7 is anticipated to inhibit phosphorylation of Akt and/or ERK1/2 in the presence of a CCN ligand. Accordingly, the agent may reduce the amount of phospho-Akt (pAkt) and/or phospho-ERK1/2 (PERK1/2) (i.e., the amount of pAkt and/or pERK1/2 will be lower in the presence of a candidate agent than the amount of pAkt and/or pERK1/2 in the untreated cells or population of cells and/or in the presence of a control agent).

Another aspect of the invention provides a method of screening for an agent that inhibits the expression of PTK7, where said agent inhibits CCN ligand-induced signalling comprising:
(i) providing a cell expressing PTK7;
(ii) contacting said cell with one or more candidate agents;
(iii) determining whether said one or more candidate agents inhibit expression of PTK7;
(iv) selecting a candidate agent that inhibits expression of PTK7 from said one or more candidate agents;
(v) contacting a cell expressing PTK7 with the candidate agent that inhibits expression of PTK7; and
(vi) determining whether the candidate agent inhibits CCN ligand-induced signalling.

In some embodiments, step (i) comprises providing one or more cells expressing PTK7.

The method may be carried out in the presence of a control agent. For example, step (ii) may also comprise contacting said cell expressing PTK7 with a control agent. Step (vi) may also comprise contacting a cell expressing PTK7 with a control agent. When the method further comprises a control agent, the method may also comprise providing more than one cell expressing PTK7.

In some embodiments, PTK7 expression is determined by measuring the surface expression of PTK7 on a cell. For example, surface expression of PTK7 on a cell that has been treated with the candidate agent can be compared with the surface expression of PTK7 on a cell that has been treated with a control agent. It is anticipated that the agent capable of inhibiting expression of PTK7 will reduce the surface levels of PTK7. Suitable methods for measuring PTK7 surface expression include assays utilizing antibodies or antibody-like molecules that recognize PTK7, e.g., flow cytometry, Western blotting or ELISA.

It is also anticipated that inhibiting expression of PTK7 inhibits CCN ligand-induced signalling and therefore inhibition of phosphorylation of Akt and/or ERK1/2 in the presence of a CCN ligand. Accordingly, the agent may reduce the amount of phospho-Akt (pAkt) and/or phospho-ERK1/2 (pERK1/2) (i.e., the amount of pAkt and/or pERK1/2 will be lower in the presence of a candidate agent than the amount of pAkt and/or pERK1/2 in the untreated cells or population of cells and/or in the presence of a control agent).

An aspect of the invention provides a method of screening for an agent that binds to a GPC (i.e., GPC1, GPC2, GPC3, GPC4, GPC5, or GPC6) and inhibits GPC-CCN ligand-PTK7 complex formation, where said agent inhibits CCN ligand-induced signalling, comprising:
(i) providing a GPC or a fragment thereof;
(ii) contacting said GPC or fragment thereof with one or more candidate agents:
(iii) determining whether the one or more candidate agents bind to said GPC or fragment thereof;
(iv) selecting a candidate agent that binds to said GPC or fragment thereof from said one or more candidate agents;
(v) providing a cell expressing a GPC;
(vi) contacting said cell with the candidate agent that binds to a GPC or a fragment thereof; and
(vii) determining whether the candidate agent inhibits CCN ligand-induced signalling.

The method may be carried out in the presence of a control agent. For example, step (ii) may also comprise contacting said GPC or fragment thereof with a control agent. Step (vi) may also comprise contacting a cell expressing a GPC with a control agent. When the method further comprises a control agent, the method may also comprise providing more than one GPC or fragment thereof and/or providing more than one cell expressing a GPC.

Candidate agent binding to a GPC may be determined by SPR or thermal shift assay. In some embodiments, formation of a GPC-CCN ligand-PTK7 complex may be measured. It is expected that formation of the GPC-CCN ligand-PTK7 complex results in CCN ligand-induced signalling via PTK7. Accordingly, formation of the GPC-CCN ligand-PTK7 complex can be measured by measuring PTK7 signalling. Thus, in some embodiments, formation of the GPC-CCN ligand-PTK7 complex is detected by measuring phosphorylation of Akt and/or phosphorylation of ERK1/2. For example, pAkt and/or pERK1/2 can be measured in a cell that has been treated with the candidate agent can be compared with pAkt and/or pERK1/2 levels in a cell in the untreated cell or population of cells or that has been treated with a control agent.

The method also provides a method of screening for an agent that inhibits the expression of a GPC (i.e., GPC1, GPC2, GPC3, GPC4, GPC5, or GPC6) comprising:
(i) providing a cell expressing a GPC;
(ii) contacting said cell with one or more candidate agents;
(iii) determining whether said one or more candidate agents inhibit expression of a GPC;
(iv) selecting a candidate agent that inhibits expression of a GPC from said one or more candidate agents;
(v) contacting a cell expressing a GPC with the candidate agent that inhibits expression of a GPC; and
(vi) determining whether the candidate agent inhibits CCN ligand-induced signalling.

In some embodiments, step (i) comprises providing one or more cells expressing PTK7 or fragment thereof.

The method may be carried out in the presence of a control agent. For example, step (ii) may also comprise contacting said cell expressing a GPC with a control agent. Step (vi) may also comprise contacting a cell expressing a GPC with a control agent. When the method further comprises a control agent, the method may also comprise providing more than one cell expressing a GPC.

In some embodiments, expression of a GPC is determined by measuring the surface expression of a GPC on a cell. For example, surface expression of a GPC on a cell that has been treated with the candidate agent can be compared with the surface expression of a GPC on a cell in the untreated cells or population of cells or that has been treated with a control agent. It is expected that the agent capable of inhibiting expression of a GPC will reduce the surface levels of a GPC. Suitable methods for measuring a GPC surface expression include assays utilizing antibodies or antibody-like molecules that recognize PTK7, e.g., flow cytometry, Western blotting, or ELISA. In some embodiments, the GPC is GPC1 or GPC4.

It is also anticipated that inhibiting expression of a GPC inhibits CCN ligand-induced signalling and therefore inhibition of phosphorylation of Akt and/or ERK1/2 in the presence of a CCN ligand. Accordingly, the agent may reduce the amount of phospho-Akt (pAkt) and/or phospho-ERK1/2 (PERK1/2) (i.e., the amount of pAkt and/or pERK1/2 will be lower in the presence of a candidate agent than the amount of pAkt and/or pERK1/2 in the untreated cells or population of cells and/or in the presence of a control agent).

For all methods described herein, the method may be performed in the presence of a CCN ligand (i.e., CCN ligand-induced signalling is measured in the presence of a CCN ligand, such as an exogenous CCN ligand). Thus, in some embodiments, step (ii) further comprises contacting the cell or population of cells with a CCN ligand. In some embodiments, step (ii) further comprises contacting the first cell or population of cells and the second cell or population of cells with a CCN ligand.

For all methods described herein, suitable methods for analysing pAKT or pERK1/2 levels in treated cells include immunoassays that can be used to measure their relative levels between groups, typically antibody-based methods such as e.g., Western blotting, alpha-assays or homogenous time-resolved FRET (HTRF) assays or non-antibody-based methods such as cells expressing kinase biosensors for the relevant kinases.

The control agent as referred to herein may be any suitable control agent, typically the same solvent or solution that is used for the candidate agent but without the candidate agent present.

The screening methods disclosed herein can be used for screening for candidate agents that can be used in the treatment or prophylaxis of fibrosis, metabolic disease, inflammatory disease, autoimmune disease, cancer, retinal disease, muscular dystrophy, cardiac disease, or polycystic kidney disease. In particular, the screening methods can be used for identifying new candidates for treating fibrosis.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organisational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Materials and Methods

### Production of Recombinant, Human C-Terminal (Domains III-IV) CCN2

In this example, the production of a C-terminal fragment (domains III-IV) of human CCN2 comprising amino acids 197-349 of CCN2 is disclosed. A DNA sequence encoding the amino acid 1-25 (signal peptide) fused in-frame to a DNA sequence encoding amino acids 197-349 of human CCN2 (uniprot numbering) was synthesized, expressed in mammalian cells, and purified as disclosed below.

The DNA sequence encoding the amino acids of CCN2 described above was codon optimized for protein expression in hamster cells (by the algorithm of the commercial supplier), a KOZAK sequence for efficient translation was appended at the 5′ end, a STOP-codon was introduced at the 3′ end and GateWay cloning AttB sites were appended on either end before the sequence was synthesized and sequence verified by a commercial supplier. The synthesized sequence was recombined with pDonrZeo by BP Gateway recombinase cloning to generate an Entry vector. Following transfection of competent E. coli mutated to allow for efficient propagation of plasmids (One Shot Top10™ cells), the entry vector was isolated with standard plasmid isolation techniques through use of a QIAprep™ Spin Miniprep kit from Qiagen™. Following plasmid isolation, the entry vector was verified by restriction enzyme digestion followed by DNA gel electrophoresis according to standard techniques well known to the skilled person. The Entry vector was further recombined with a destination vector using LR gateway recombinase. The destination vector used was pUCOE-DHFR-DEST, as described by Kaasbøll et al., (J. Biol. Chem., 293 (46): 17953-17970, 2018).

Following transfection of competent E. coli, mutated to allow for efficient propagation of plasmids (One Shot Top10™ cells), the expression vector was isolated with standard plasmid isolation techniques using a QIAGEN™ Plasmid Plus Maxi Kit. The resulting expression vector was verified by standard restriction enzyme digestion and DNA gel electrophoresis according to standard techniques well known to the skilled person. The resulting expression vector was then transferred into ExpiCHO suspension culture adapted CHO cells according to the “Creation and Scale up of a Stable Cell Line Using ExpiCHO™ Products” protocol supplied by the manufacturer of the ExpiCHO™ Stable Production Medium (Gibco Cat. #: A3711001). The cells were maintained in vented Erlenmeyer flasks in cell culture incubators kept at 37° C. with 8% CO2 on a shaker platform (as described in Kaasbøll et al., supra). The transfected cells were kept overnight in ExpiCHO™ Expression medium before being transferred to ExpiCHO™ expression medium supplemented with 0.1 μM methotrexate. The cells were then sub-cultured until the viability again approached 80%, at which point the medium was supplemented with 1 μM methotrexate. The cells were again sub-cultured until the viability exceeded 30% at which point the cells were transferred to ExpiCHO™ Stable Production Medium supplemented with 1 μM methotrexate and further sub-cultured until the viability exceeded 95% and the doubling-time decreased to less than 20 hours, at which point the cell pool was considered stably transfected.

Once the stable cell pool was established the cell culture volume was expanded to allow for the seeding of stably transfected cells for production at a density of 1*10\hat{ }6 cells/mL, which was supplemented daily with 5% (v/v of the starting volume) with 2× EfficientFeed™ C+ supplement. The cell culture was harvested 12 days after seeding when viabilities were >95% by centrifugation at 4750 g. The harvested cell culture supernatant was treated aseptically, supplemented with MES from 1M stock to a final concentration of 50 mM, PMSF, from a stock of 0.1 M in 2-propanol, to a final concentration of 0.1 mM, L-Arginine from a stock of 2 M and pH of 4.0 to a final concentration of 100 mM and EDTA, from a stock of 0.5M, pH 8.0 to a final concentration of 2 mM.

The expressed C-terminal fragment of human CCN2 was subsequently purified from the harvested cell culture supernatant by ion-exchange (HiTrap 5 mL, HP SP, GE HealthCare column) and size exclusion chromatographic techniques. Briefly, the sample was split into two samples of 800 mL each and in two consecutive runs loaded onto a HiTrap 5 mL, HP SP (GE HealthCare) column pre-equilibrated with buffer A (150 mM NaCl, 100 mM L-Arginine, 50 mM MES, pH 6.0) connected to a FPLC chromatography system (BioRad NGC). The column was washed with buffer A and eluted with a gradient elution with buffer A and buffer B (2M NaCl, 100 mM L-Arginine, 50 mM MES, pH 6.0). Fractions were analysed by SDS-PAGE utilizing StainFree™ (BioRad) gels imaged on a ChemiDoc imaging system (Bio-Rad) and fractions containing bands corresponding to the expected size of the C-terminal fragment from both runs were pooled to generate the sample for the size exclusion step. The pooled fractions were loaded onto a HiScale 26-40 Superdex200Inc size exclusion column (GE HealthCare) pre-equilibrated with buffer A2 (100 mM Na_{1/2}H_{1/2}PO₄, 300 mM NaCl, pH 6.5), the column was eluted with buffer A2, the eluted fractions were analysed by SDS-PAGE utilizing StainFree™ (BioRad) gels imaged on a ChemiDoc imaging system (Bio-Rad), the main peak on the chromatogram was confirmed to contain the C-terminal fragment of human CCN2 and thereafter pooled and sterile-filtered.

The concentration of the purified C-terminal fragment of human CCN2 was determined with a micro BCA™ protein assay kit (ThermoFisherScientific) according to manufacturer's instructions and the potency of the preparation determined by stimulation of a Rat2 fibroblast cell line stably transfected with a kinase biosensor for Akt as described in Kaasbøll et al (J. Biol. Chem., 293 (46): 17953-17970, 2018).

### HATRIC-LRC (LRC-TriCEPS) Experiments

Cell culture: For the first experiment RAT2, rat fibroblast cells (TebuBio, #IFO50282) were cultured in DMEM supplemented with 2 mM Glutamine, 5% Foetal Bovine Serum (FBS) and Pen/Strep following the manufacture's recommendations. For the LRC-TriCEPS experiment, for each treatment arm, 5×150 cm² dishes of nearly 80% confluent Rat2 cells were used.

For the second experiment HEK293A cells passage 4 (originally from ThermoFisher, #R70507) were cultured in DMEM supplemented with 0.1 mM Non Essential Amino Acids (NEAA), 2 mM Glutamine and 10% Foetal Bovine Serum (FBS) following the manufacture's recommendations. For the LRCTriCEPS experiment, for each treatment arm, 5×175 cm² flasks of nearly 90% confluent 293A cells passage 5 were used.

Ligand coupling: The purified C-terminal fragment of human CCN2 (ligand) was dialyzed overnight in PBS pH 7.4 using a Slide-A-Lyzer MINI dialysis device, 3.5K MW cut-off to allow for optimal ligand-TriCEPS v3.0 coupling. The ligand-TriCEPS v3.0 coupling was performed by using 300 μg of ligand and 150 μg of TriCEPS v.3.0 following the manufacturer's (Dualsystems Biotech) standard conditions.

Receptor capture for LRC-TriCEPS: For the experiment utilizing Rat2 cells receptor capturing was performed on 80% confluent 150 cm² dishes of Rat2 cells. Cells were previously oxidized with 1.5 mM of sodium metaperiodate for 15 min in PBS pH 6.5 at 4° C. For each ligand, 5×150 cm² dishes containing Rat2 cells were used. 300 μg of each TriCEPS coupled ligand was added to 20 ml PBS pH6.5 and 4 ml was added to each dish. LRC-TriCEPS experiment was performed at pH 6.5, 4° C., for 90 minutes.

For the experiment utilizing HEK293A cells receptor capturing was performed on ca. 90% confluent 15 cm dish of 293A cells. Cells were previously oxidized with 1.5 mM of sodium metaperiodate for 15 min in PBS pH 6.5 at 4° C. For each ligand, 5×150 cm² dishes containing 293A cells were used. 300 μg of each TriCEPS coupled ligand was added to 15 ml PBS pH6.5 and 3 ml were added to each dish.

LRC-TriCEPS experiment was performed at pH 6.5, 4° C., for 90 minutes. After the incubation time, cells were harvested by scraping and cells of each treatment arm were divided into three tubes to process the samples in triplicate.

Sample preparation: Cells were lysed according to the standard protocol and target proteins were purified using solid phase chromatography. After stringent washing steps to remove unspecific interactions, proteins were reduced, alkylated, and digested with trypsin. The tryptic peptides were collected for LC-MS/MS.

Mass spectrometry: The LRC-TriCEPS samples were analysed on a Q-Exactive mass spectrometer (Thermo-Fisher Scientific) fitted with an electrospray ion source. Tryptic peptides were measured in data dependent acquisition mode (TOP12) in a 90 min gradient using a 50 cm C18 packed column.

Data analysis: The Progenesis software was used for raw file alignment and feature detection, Comet search engine was used for spectra identification and the Trans proteomic pipeline was used for statistical validation of putative identifications and protein inference. Upon protein inference, relative quantification of control and ligand samples was performed based on ion extracted intensity and differential protein abundance was tested using a statistical ANOVA model followed by multiple testing correction. This model assumes that the measurement error follows Gaussian distribution and views individual features as replicates of a protein's abundance and explicitly accounts for this redundancy. It tests each protein for differential abundance in all pairwise comparisons of ligand and control samples and reports the p-values. Next, p-values are adjusted for multiple comparisons to control the experiment-wide false discovery rate (FDR).

In experiment utilizing Rat2 cells, the rat proteome database from Uniprot (Rattus norvegicus) including the sequence of human CCN2 and BSA was used for analysis. In the experiment utilizing HEK293A cells human proteome database from Uniprot including the sequence of BSA was used for analysis.

One of the ligands of interest samples from the Rat2 experiment did not align with the other samples and therefore was excluded for further analysis.

### Evaluation of the Results:

The criteria to consider a protein as a candidate for interacting to the ligand of interest are the following:

$$Log 2 fold change > 2 = fold change > 4. \qquad 1$$

$$- Log 10 ( adjust p - value ) > 2 = adjusted p - value ( q - value ) < 0.01 . \qquad 2$$

3. At least 2 peptides identified of the protein

### TriCEPS-Flow-siRNA Experiments

Cell culture: Rat2, rat fibroblast cells (TebuBio, #IFO50282) were cultured in DMEM supplemented with 2 mM Glutamine, 5% Foetal Bovine Serum (FBS) and Pen/Strep following the manufacture's recommendations.

siRNA reverse transfection of Rat 2 fibroblasts in 12 well plates: All steps were performed in a cell culture hood using sterile techniques.

Transfection mix: Reagents used include Scramble siRNA (On-Target plus non-targeting control pool, #D-001810-10-5, Horizon Discovery), siRNA against rat PTK7 (On-Target plus Rat Ptk7 301242, #J-082417-06-0002 and #J-082417-07-0002, Horizon Discovery), and Dharma FECT1 transfection reagent (Horizon Discovery).

For the transfection mix, 5 μL of siRNA stock (5 μM) was diluted in 95 μL serum-free medium and incubated for 5 minutes at room temperature. 2 μL of dharmaFECT 1 (Horizon Discovery) was diluted in 98 μL serum-free medium. The diluted siRNA was added to the diluted dharmaFECT, mixed by carefully pipetting up and down and incubated for 20 minutes at room temperature, before 200 μL of the mixture was added per well and incubated for 30 minutes at room temperature.

Cells were trypsinized, counted and diluted in antibiotic-free complete medium (DMEM supplemented with 2 mM Glutamine, 5% FBS) to a density of 75 000 cells/well and a final volume per well of 1000 μL. The cells with the transfection mixture were then incubated for 24 hours, 48 hours or 72 hours prior to the Flow-TriCEPS experiments or RNA-extraction-qPCR experiments.

RNA extraction: For RNA extraction a PureLink RNA Mini Kit (#12183018A. Invitrogen) was used and a step for DNase digestion was included (PureLink DNase 12185010, Invitrogen). Briefly, RNA was extracted 24-, 48- or 72-hours post-transfection, depending on the experiment. First cells were collected from 12 well plate in 0.3 ml/well of PureLink Lysis Buffer containing 40 μM DTT. The lysate was transferred to a homogenizer (12183-026, Invitrogen) inserted in a collection tube and centrifuged at 12000 g for 2 minutes at room temperature (RT). One volume (300 μl) 70% EtOH (in RNase free water) was added to one volume of cell homogenate and vortexed. The sample (600 μl) was transferred to the Spin Cartridge (with the collection tube) and centrifuged at 12000 g for 2 minutes at RT. Flow-through was discarded. 350 μl Wash Buffer I (from the kit) was added to the Spin Cartridge containing bound RNA and centrifuged at 12000 g for 15 seconds at RT. Flow-through was discarded and Spin Cartridge inserted into a new collection tube. 80 μl DNase mixture (Table 2) was added directly onto the surface of the Spin Cartridge membrane and incubated for 15 minutes at RT.

PureLink DNase mixture (Table 2):

| | Volume per |
|---|---|
| Component | sample (μl) |
| 10x DNase I Reaction Buffer | 8 |
| Resuspended DNase | 10 |
| RNase-free water | 62 |
| Final volume | 80 |

Following DNase treatment 350 μl Wash Buffer I was added to the Spin Cartridge and centrifuged at 12000 g for 15 seconds at RT. Flow-through was discarded. Then 500 μl Wash Buffer II (from the kit) was added to the Spin Cartridge and centrifuged at 12000 g for 15 seconds at RT. Flow-through was discarded. This washing step was repeated once. The Spin Cartridge was centrifuged for 1 minute to dry the membrane with bound RNA, collection tube discarded, and Spin Cartridge inserted into a recovery tube. 30 μl RNase-free water was added to the center of the Spin Cartridge, incubated for 1 minute at RT before centrifugation at 12000 g for 1 minute at RT. Next RNA concentration was measured by

NanoDrop and samples kept at −20° C. till use. cDNA synthesis: The qScript cDNA Synthesis Kit (#95047, Quantabio) was used. Dilutions of RNA was calculated to ensure the same amount went into the mix while making sure to keep the amount within the range of the kit. Total amounts RNA varied between 280-500 ng in the experiments. All samples were placed on ice an into PCR tubes and reaction mixes according to Table 3 added to the tubes.

TABLE 3
cDNA synthesis mixtures

| | | |
|---|---|---|
| | variable | variable |
| RNA (from 280-500 ng) | (max. 15 μl) | (max. 15 μl) |

| | | |
|---|---|---|
| Nuclease-free water | variable | variable |

| qScript Reaction Mix (5X) | 4 | μl | 4 | μl |
|---|---|---|---|---|
| qScript RT (20X) | 1 | μl | — | |
| final volume | 20 | μl | 20 | μl |

A control for contamination with genomic DNA was included by means of RNA containing reactions that did not have the reverse transcriptase (qScript) included in the reaction mixture. The following thermal-cycling conditions were used:
Step 1:5 min, 22° C., 1 cycle;
Step 2:30 min, 42° C., 1 cycle;
Step 3:5 min, 85° C., 1 cycle,
Step 4:4° C.

After cDNA synthesis the samples were kept at −20° C. till use.

qRT-PCR: The following reagents were used: TaqMan Fast Advanced Master Mix (#4444556, Thermo Fisher Scientific), TaqMan Gene Expression Assays: FAM-PTK7 Rat (#Rn01757096_m1, ThermoFisher) and FAM-GAPDH Rat (#Rn01775763_g1, ThermoFisher), MicroAmp Optical 96-well plate reaction plate (Applied Biosystems, #N8010560). MicroAmp Optical Adhesive Film (Applied Biosystems, #4311971). A common master mix was prepared (Table 4) and each reaction was performed in a total volume of 20 μl in a 96 well plate format in triplicates.

TABLE 4
qRT-PCR reactions

| | Volume (μl) |
|---|---|
| Component | for 1 reaction |
| TaqMan Fast Advanced Master Mix (2X) | 10 |
| TaqMan Gene Expression Assay (20X) | 1 |
| Nuclease-free water | 7 |
| Total volume per reaction | 18 |

For each synthesized cDNA, 2 μl was pipetted into a 96 well plate in triplicates and 18 μl of the common master mix was added per sample. The reaction plate was covered with an optical adhesive film and centrifuged to spin down the contents and eliminate air bubbles (100 g for 20 seconds). The following thermal-cycling conditions were used:
Step 1:2 min, 50° C., 1 cycle;
Step 2:20 sec, 95° C., 1 cycle;
Step 3:1 sec, 95° C., 20 sec, 60° C., 40 cycles.

Ligand labelling: 20 μg of each ligand was coupled to 10 μg of TriCEPS v.2.0 in a total volume of 50 μl PBS pH7.4. All samples were quenched with 20 μg of glycine after coupling to prevent non-specific binding of TriCEPS to the cell surface and reduce the background signal.

Ligand-cell incubation and flow cytometric analysis: For each sample a well of a 12 well plate containing the corresponding cell line (at about 50-60% of confluency) was treated with 3 μg of ligand coupled to TriCEPS v.2.0 in 400 μl PBS (pH7.4 or pH6.5, depending on the condition tested). After the corresponding incubation time, cells were washed twice with 400 μl ice cold PBS (pH7.4 or pH6.5 respectively) and labelled with streptavidin-R-PE. Afterwards, cells were collected by scraping, washed twice with 400 μl of ice-cold PBS (pH7.4 and pH6.5 respectively), and analysed by flow cytometry.

### Ligand-Binding Assays

TABLE 5
Instruments and reagents:

| | |
|---|---|
| DuoSet Ancillary | BioTechne: catalog number DY007. |
| Reagent Kit 1 | lot: P297274. exp: 2022 Oct. 25 |
| Streptavidin- | BioTechne. Cat no 890803, lot: P272052. |
| HRP A | exp: 2025 Dec. 12 |
| PBS | Lonza. Cat no:17-512F, lot 0001023463, |
| | exp 2023 Jul. 12 |
| Plate shaker | OHAUS Orbital Shaker |
| Plate reader | Thermo Scientific; Multiscan GO |
| d34CCN2 | C-terminal human CCN2 (purification |
| | described above) |
| PTK7-HIS | Acrobiosystems, cat no: PT7-H52H3. |
| | lot: 4313a-217XF1-XP |
| GPC1-HIS | AcroBiosystems, cat no: GP1-H2H9; |
| | cat no: 3167a-97CF1-RW |
| GPC4-Fc | R&D, cat no: 9195-GP-050. |
| | lot: DFEE0320011 |
| CaptureSelect ™ | ThermoFisherScientific, |
| Biotin Anti-IgG- | Catalog number: 7103262100 |
| Fc (Human) | |
| Conjugate | |
| His Tag | RnD cat no: BAM050, |
| Biotinylated | lot no: DXN130061 |
| Antibody | |

Buffers were prepared according to manufacturer's instructions.

96-well plates (from kit) were coated with 100 μL d34CCN2 diluted in coating buffer and left at 4° C. overnight. The following day, the wells were washed 4 times with 300 μL wash buffer and the plate blotted on paper before 300 μL blocking buffer was added to the wells and the plate incubated at >1 hour at room temperature. After blocking the wells were washed 4 times with wash buffer before blotting on paper. Afterwards 100 μL of the test proteins diluted in reagent dilution buffer (kit) in a range of concentrations were added to the wells and the plate incubated for 1 hour at 37° C. Next the wells were washed 4 times with 300 μL wash buffer and the plate blotted on paper. Then 100 μL CaptureSelect™ Biotin Anti-IgG-Fc (Human) Conjugate or His-tag Biotinylated antibody diluted to 500 ng/ml in reagent dilution buffer was added to the wells incubated with Fc- or His-tagged recombinant proteins respectively and incubated for 1 hour at 37° C. Next the wells were again washed 4 times with 300 μL wash buffer and the plate blotted on paper before addition of 100 μL Streptavidin-HRP diluted 1:200 (v/v) in reagent diluent 1 to and incubated for 20 minutes at 37° C. Again, the wells were washed 4 times with 300 μL wash buffer and blotted on paper before the addition of 100 μL substrate solution and incubation at room temperature for 20 minutes (GPC1-His and PTK7-His) or 15 minutes (GPC4-Fc) prior to addition of 50 μL stop solution. The stop solution was blended with the substrate solution by light tapping of the plate. The absorbance was recorded at 450 nm.

### Ligand-Trap-Phosphoprotein Assays

TABLE 6
Instruments, reagents, and cell lines:

| | |
|---|---|
| Phospho-AKT 1/2/3(ser473) | Cat no: 64AKSPEG, lot: 17E. |
| kit Cisbio (500 test kit) | Exp date: 2023 Feb. 12. Cisbio |
| Alpha Seal adhesive sealing | 6050185 PerkinElmer. |
| film | |
| HTFR 96 well detection plate | Cat no: 66PL96025 |
| Centrifuge | MEGA STAR 1.6 R VWR |
| Plate shaker | OHAUS Orbital Shaker |
| Cell line | Rat 2 fibroblasts |
| Medium | DMEM with 10% serum (ThermoFisherScientific |
| | Ref: 16000-044, lot no: 2208592RP. Exp 2025 Feb. |
| | 28 Heat inactivated 30 minutes at 56° C.) |
| | and Gensumycin. Starvation Medium: Gibco, DMEM |
| | without Phenol Red, ref: 31053-028, lot 2375246; |
| | exp 2022 August + 0.5 ml Gentamicin reagent |
| | Solution, Gibco, ref 15750-060, exp 2023 Apr. 30. |
| Accutase | BioWest L0950, Accutase Batch MSOOXH1, exp: |
| | Jan. 12, 2022 |
| PBS: | Lonza. Cat no:17-512Q, lot: 0000891727 Expire date: |
| | 2022 Jun. 04. |
| EGF | R&D Systems. Cat no: 236-EG, lot no: HLM8721042, |
| | reconstituted in PBS |
| d34CCN2 | C-terminal human CCN2 (purification described above) |
| GPC1-His | R&D Cat no: 4519-GP-050; QNG0421061. 50 μg |
| PTK7-Fc | RnD cat no: 9799-TK-050; lot DHMC0221032. 101 kDa |
| Tissue-culture treated | Corning Costar ref 3595, lot 13421019, |
| plates | exp 2024 May 13. |
| Cell counter | AMQAX2000, COUNTESS 3, Invitrogen/ |
| | ThermoFisherScientific |
| CO2 incubator set at | New Brunswick Galaxy 170S. ShareLab I/C1.3 |
| 5% CO2 | |
| Plate reader | Envision, PerkinElmer |
| Pipettes: | P2; P4; P5; P7; P10; P11 |

Rat 2 cells were detached with Accutase, counted, and seeded out at a density of 9000 cells/well in a 96 well plate, placed in the incubator and left overnight. The following day the wells were washed twice with 100 μL medium without phenol red and 100 μL/well of medium without phenol red was added before the plates were placed in the incubator and left overnight.

The following day 60 μl medium was removed from each well and 10 μL of the stimulants diluted in PBS were added to the wells and incubated for 15 minutes in the incubator. After 15 minutes 20 μl of the stimulation solutions were removed by pipetting and 30 μl of 2× lysis buffer with freshly prepared blocking reagent was added to each well. The plate was incubated with shaking (350 rpm) at room temperature for 45 minutes before being placed in the freezer at −80° C. for 30 minutes. Next the plates were thawed, incubated with shaking (350 rpm) at room temperature for 10 minutes, the lysate was pipetted up and down with a multichannel pipette, the plates were centrifuged for 5 minutes at 2000 g before 16 μL of the supernatant was transferred to a HTFR 96 well detection plate.

Working solutions of the antibodies were prepared by dilution in detection buffer (1:20 v/v) and mixed (1:1 v/v) just prior to addition to the wells with the lysate (4 μL/well). The plate containing lysate and antibody mix was then sealed, shaken for 4 minutes on a plate shaker (200 rpm), incubated overnight at room temperature in a plastic bag with wet paper tissues, and the following day read on the plate reader.

### SIRNA—Phosphoprotein Assays

TABLE 7
Instruments, reagents and cell lines:

| | |
|---|---|
| Phospho-AKT 1/2/3(ser473) | Cat no: 64AKSPEG, lot: 17F. |
| kit Cisbio (500 test kit) | Exp date: 2023 Feb. 12. Cisbio |
| GAPDH Housekeeping kit Cisbio | Cat no: 64GAPDHPEG, aliquoted |
| | 2022 Apr. 19 |
| Alpha Seal adhesive sealing | 6050185 PerkinElmer. |
| film | |
| HTFR 96 well detection plate | Cat no: 66PL96025 |
| Centrifuge | MEGA STAR 1.6 R VWR |
| Plate shaker | OHAUS Orbital Shaker |
| Cell line | Rat 2 fibroblasts |
| Medium: DMEM with 5% serum | DMEM: GIBCO 41965-039; lot 2418636, |
| without antibiotics. | exp 2022 Oct. 31 FBS: ThermoFisherScientific |
| | Ref: 16000-044, lot no: 2314116RP. |
| | Exp 2026 Jan. 30, Heat inactivated 30 minutes |
| | at 56° C. |
| Starvation medium: | Gibco, DMEM without Phenol Red, ref: 31053-028, |
| | lot 2375246, exp 2022 August + 0.5 ml Gentamicin |
| | reagent Solution, Gibco, ref 15750-060, |
| | exp 2023 Apr. 30. + GlutaMAX, ref A12860-01; |
| | lot: 2312192. Exp: 2023 Feb. 28 |
| Accutase | BioWest L0950, Accutase Batch MSO16A1, |
| | exp: Jan. 12, 2022 |
| PBS: | Lonza. Cat no: 17-512Q, lot: 000023463 |
| | Expire date: 2022 Jun. 4. |
| EGF | R&D Systems. Cat no: 236-EG, |
| | lot no: HLM8721042, reconstituted in PBS |
| DharmaFECT 1 Transfection | Horizon Discovery Cat no: T-2001-01. |
| Reagent, 0.2 mL | Lot: 01180193 |
| ON-TARGETplus Non-targeting | Horizon Discovery Cat no: D-001810-10-05. |
| Pool, 5 nmol | Lot: 7595387B2 |
| ON-TARGETplus Rat Ptk7 | Horizon Discovery Cat no: J-082417-06-0002. |
| (301242) siRNA - | Lot: 220304 |
| Individual, 2 nmol | |
| ON-TARGETplus Rat Ptk7 | Horizon Discovery Cat no: J-082417-07-0002. |
| (301242) siRNA - | Lot: 220304 |
| Individual, 2 nmol | |
| d34CCN2 | C-terminal human CCN2 (purification |
| | described above) |
| Tissue-culture treated | Corning Costar ref 3595, lot 13421019, |
| plates | exp 2024 May 13. |
| Cell counter | AMQAX2000, COUNTESS 3, Invitrogen/ |
| | ThermoFisherScientific |
| CO2 incubator set at | New Brunswick Galaxy 170S. ShareLab I/C1.3 |
| 5% CO2 | |
| Plate reader | Envision, PerkinElmer |
| Pipettes: | P2; P4; P5; P7; P10; P11 |

Transfection mixtures were prepared in a sterile hood. First siRNA stock solutions dissolved in double-dietiled, sterile-filtered H2O were diluted in sorum-free DMEM to yield a concentration of 250 nM, secondly DharmaFECT 1 transfection reagent was diluted 1:50 in serum-free DMEM. The diluted siRINAs and DharmeFECT 1 transfection reagents were incubated at 5 minutes at room temperature before they were mixed (1:1 v/v) and incubated for 20 minutes at room temperature. 20 μL of the resulting transfection mixtures were added to the tissue-culture treated 96-well plates.

Rat2 cells were detached with Accutase, resuspended in DMEM with 5% serum without antibiotics, passed through a cell strainer, counted, and diluted to make a cell suspension of 94 000 living cells/mL. 80 μL of the cell suspension was added to the wells pre-incubated with the transfection mixtures. The plates were then incubated at 37° in the CO2 incubator for 48 hours. After 48 hours the wells were washed twice with 100 μL starvation medium and 90 μL/well of starvation medium was added to each well and the plates placed in the CO2 incubator overnight. The following day 10 μL of the stimulants diluted in PBS were added to the wells and incubated for 15 minutes in the incubator. After 15 minutes 70 μl of the stimulation solutions were removed by pipetting and 30 μl of 2× lysis buffer with freshly prepared blocking reagent was added to each well.

The plate was incubated with shaking (350 rpm) at room temperature for 45 minutes before being placed in the freezer at −80° C. for 30 minutes. Next the plates were thawed, incubated with shaking (350 rpm) at room temperature for 10 minutes, the lysate was pipetted up and down with a multichannel pipette, the plates were centrifuged for 5 minutes at 2000 g before 16 μL of the supernatant was transferred to a HTFR 96 well detection plate. Working solutions of the antibodies were prepared by diluted in detection buffer (1:20 v/v) and mixed (1:1 v/v) just prior to addition to the wells with the lysate (4 μL/well). The plate containing lysate and antibody mix was then sealed, shaken for 4 minutes on a plate shaker (200 rpm), incubated overnight at room temperature in a plastic bag with wet paper tissues, and the following day read on the plate reader.

### SiRNA—Phosphoprotein/Total Protein Assays

Transfection mixtures containing transfection reagent DharmFECT 1 (Horizon, T-2001-02) and ON-TARGETplus siRNAs directed towards PTK7, non-targeting control (NTC) siRNA (25 nM) (both from Horizon Discovery) or transfection reagent only were distributed to 96-well cell culture plates. Single cell suspensions of Rat2 fibroblasts were then distributed at a concentration of 4000 cells/well. Parallel plates were prepared to assess both phospho- and total protein contents, cell viabilities and knockdown efficiency. The day after seeding cell culture media was changed. Two days after cell seeding the media was changed to starvation media without serum. Three days after cell seeding the cells were treated with selected concentrations of d34CCN2 or vehicle for 5 minutes prior to harvesting for downstream analyses. For cell viabilities cells were harvested and processed with ATPLite viability assays as per manufacturer's instruction (PerkinElmer), read on a PheraStar plate reader (BMG Labtech, Germany) and results expressed as percent of non-treated control. For phospho- and total protein contents cells were harvested and processed with Advanced phospho-ERK (Thr202/Tyr204) (PERK) and total ERK (totERK) cellular kit Homogeneous Time Resolved Fluorescence (HTRF) assays (Cisbio a part of PerkinElmer) according to the manufacturer's instructions and read with a PheraStar platereader (BMG Labtech) and expressed as a pERK/totERK ratio. For knockdown efficiency cells were processed with Cells-to-Ct kit (ThermoFisher, Cat #A25603) and analyzed with TaqMan primer-probe kits for PTK7 and the housekeeping gene ACTB and QuantStudioTM7 Pro (both from Thermo Fisher). Knockdown efficiency (fold change in gene expression) was calculated with the ΔΔCq method (normalized to non-targeting control; NTC) (ΔCq=Cq_{PTk7}-Cq_{ACTB}, ΔΔCq=ΔCq_{PTK7 siRNA}-ΔCq_{NTC}, Fold change PTK7 expression=2^{-ΔΔCq}.

### Example 1: Screen for Membrane Receptors of the Active C-Terminal Fragment of CCN2

The working paradigm is that the C-terminal fragment (domains III-IV) of CCN2 engages a cell membrane receptor that engenders rapid activation of intracellular signalling cascades, such as PI3K/AKT and MAPK-signalling, while full-length CCN2 (i.e., the proprotein) does not initiate rapid cell signalling. Accordingly, the screen for signalling receptors was carried out with the C-terminal fragment (domains III-IV) of CCN2.

The principle of the screening technology employed, HATRIC-LRC, is described by Sobotzki N et al. (Nature Communications volume 9, Article number: 1519 (2018). HATRIC-LRC is a refinement of the TRICEPS-LRC technique described by Frei A P et al. (Nat Biotechnol. 2012 October; 30 (10): 997-1001).

In this experiment, the ligand of interest i.e., purified recombinant human C-terminal (domains III-IV) CCN2 (“d34CCN2”) was coupled to the trifunctional TriCEPs v3.0 molecule in parallel with control ligands, such as bovine serum albumin (BSA) or transferrin (TRFE).

Cells of choice were exposed to mildly oxidizing conditions and were then incubated with the labelled ligands in the presence of a catalyst that catalyses cross-linking of the labelled ligands to sugar-chains in the vicinity. The HATRIC molecule was subsequently “pulled-out” along with ligands and cross-linked proteins, proteins were identified by LC-MS/MS and identified proteins interrogated for enrichment in the ligand of interest samples relative to the control samples.

### Screen for Membrane Receptors of the Active, C-Terminal Fragment of CCN2

Two HATRIC-LRC experiment with purified, recombinant, human C-terminal (domains III-IV) CCN2 were performed with two different cell lines, Rat2 fibroblasts and HEK293A cells.

Criteria to consider a protein as a candidate for interacting to the ligand of interest were the following:

$$Log 2 fold change > 2 = fold change > 4. \qquad 1$$

$$- Log 10 ( adjust p - value ) > 2 = - adjusted p - value ( qvalue ) < 0.01 . \qquad 2$$

3. At least 2 peptides identified of the protein

The result of the analysis of enrichment in purified recombinant, human C-terminal (domains III-IV) CCN2 (“d34CCN2”) relative to TRFE in the Rat2 cell screen identified GPC1 and PTK7 as enriched (15 peptides identified, log 2 (FC) 4.4), q-value 3.92*10\hat{ }−4 and 11 peptides identified, log 2 (FC) 3.0, q-value 3.92*10\hat{ }−4 respectively) and relative to BSA, GPC1 and PTK7 were also found as enriched (15 peptides identified, log 2 (FC) 4.2), q-value 1.81*10\hat{ }−4 and 11 peptides identified, log 2 (FC) 2.7, q-value 1.81*10\hat{ }−4 respectively).

As an indication of technical success for the Rat2 cell screen, the TRFE receptor, TRF1, was identified, as de-enriched in the TRFE group relative to the d34CCN2 group (9 peptides identified, log 2 (FC)-2.8, q-value 2.37*10\hat{ }−3). Interestingly CCN1 was also found to be enriched in the d34CCN2 group relative to both TRFE and BSA (3 peptides identified, log 2 (FC) 4.1), q-value 2.15*10\hat{ }−4 and 3 peptides identified, log 2 (FC) 3.4, q-value 1.81*10\hat{ }−4 respectively) in the Rat2 cell screen.

The result of the analysis of enrichment in purified recombinant, human C-terminal (domains III-IV) CCN2 (“d34CCN2”) relative to TRFE in the HEK293 cell screen identified GPC1, GPC4 and PTK7 as enriched (11 peptides identified, log 2 (FC) 4.19, q-value 7.86*10\hat{ }−5, 10 peptides identified, log 2 (FC) 2.84, q-value 1.29*10\hat{ }−4 and 19 peptides identified, log 2 (FC) 3.09, q-value 1.29*10\hat{ }−5 respectively) and relative to BSA, GPC1, GPC4 and PTK7 were also found as enriched (11 peptides identified, log 2 (FC) 2.85, q-value 1.06*10\hat{ }−3, 10 peptides identified, log 2 (FC) 2.85, q-value 1.40*10\hat{ }−3 and 19 peptides identified, log 2 (FC) 3.26, q-value 2.32*10\hat{ }−4 respectively).

As an indication of technical success for the HEK293A cell screen the TRFE receptor, TRF1, was identified as de-enriched in the TRFE group relative to the d34CCN2 group (20 peptides identified, log 2 (FC)-3.35, q-value 1.49*10\hat{ }−3). Interestingly CCN1 was also found to be enriched in the d34CCN2 group relative to both TRFE and BSA (4 peptides identified, log 2 (FC) 4.73), q-value 5.25*10\hat{ }−5 and 4 peptides identified, log 2 (FC) 5.63, q-value 1.95*10\hat{ }−3 respectively) in the HEK293A cell screen.

### Example 2: Solid-Phase Ligand-Binding Assays

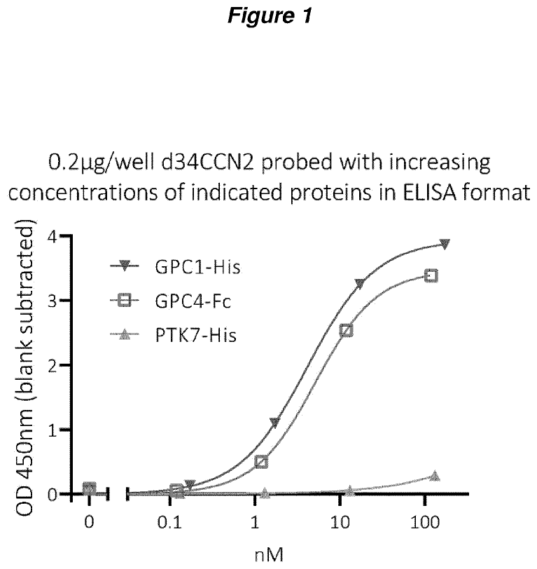

Having identified several potential membrane proteins that bind to the purified recombinant human C-terminal (domains III-IV) CCN2 in the cell-based screen, described above, solid-phase binding assays for purified recombinant human C-terminal (domains III-IV) CCN2 to recombinant versions of the extracellular domains of some of the candidates were performed. In FIG. 1, the total binding of recombinant extracellular domains of GPC1, GPC4 and PTK genetically fused to His or Fc-tags to immobilized recombinant human C-terminal (domains III-IV) CCN2 is shown.

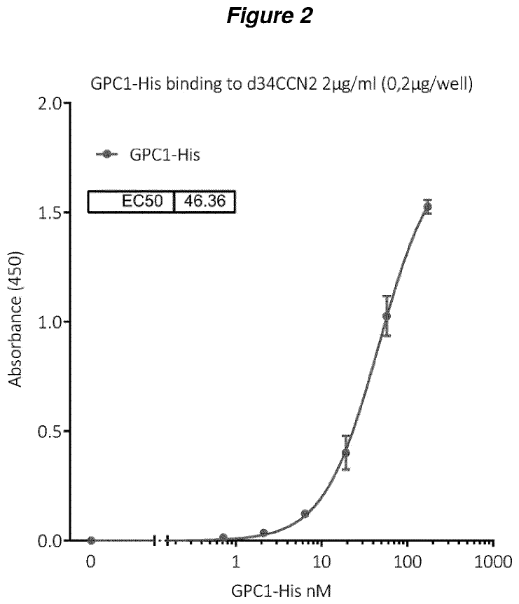

FIG. 2 shows the specific binding of GPC1 to immobilized recombinant human C-terminal (domains III-IV) CCN2.

### Example 3: Ligand-Trap Phosphoprotein Assays

A commonly used approach to block receptor-ligand interactions for receptor tyrosine kinases is the use of ligand-traps, i.e., soluble proteins comprising the extracellular domains (ECD) of the receptors in question, typically produced with recombinant DNA technology. The ligand-trap approach works both as a tool for studying ligand-receptor induced signalling and as a means of blocking a particular ligand-receptor interaction for pharmaceutical purposes, e.g., as in etanercept, luspatercept, sotatercept or aflibercept (Attwood M M et al., Nat Rev Drug Discov. 2020 October; 19 (10): 695-710.).

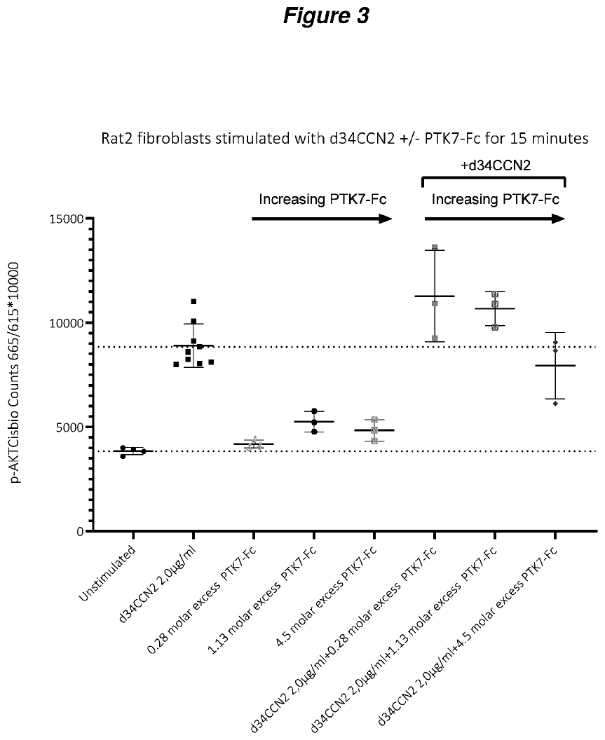

In FIG. 3 it can be seen that increasing doses of recombinant human PTK7-ECD genetically fused to an Fc-fragment (PTK7-Fc) had an endogenous stimulatory effect on the pAKT S473 levels in Rat2 fibroblasts. Furthermore, it can be appreciated that the addition of increasing concentrations of PTK7-Fc combined with a set concentration of recombinant human C-terminal (domains III-IV) CCN2 (d34CCN2) reduced the levels of pAKT S473.

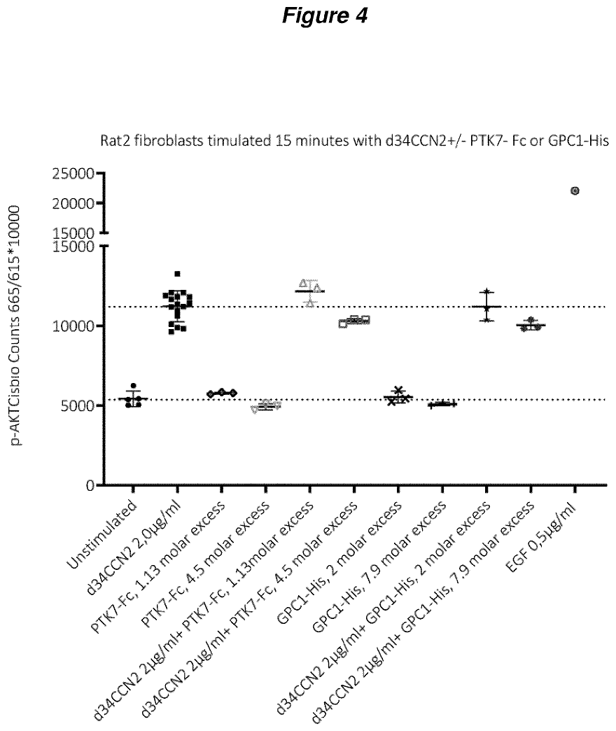

In FIG. 4 it can again be seen that recombinant PTK7-ECD genetically fused to an Fc-fragment (PTK7-Fc) inhibits a d34CCN2-stimulated increase in pAKT levels in Rat2 cells. Furthermore, it can be appreciated that also recombinant human GPC1 genetically fused to an His-tag combined with a set concentration of recombinant human C-terminal (domains III-IV) CCN2 (d34CCN2) reduced the levels of PAKT S473 in Rat2 fibroblasts.

### Example 4: SiRNA-Flow TriCEPS

To assess the functional relevance of PTK7 for binding of CCN2 to cell membranes of live cells Rat2 cells, the receptor expression in Rat2 fibroblasts was modulated by siRNAs either targeting PTK7 or not (scrambled control) and subsequently binding of recombinant human C-terminal (domains III-IV) CCN2 to the cell membranes were investigated.

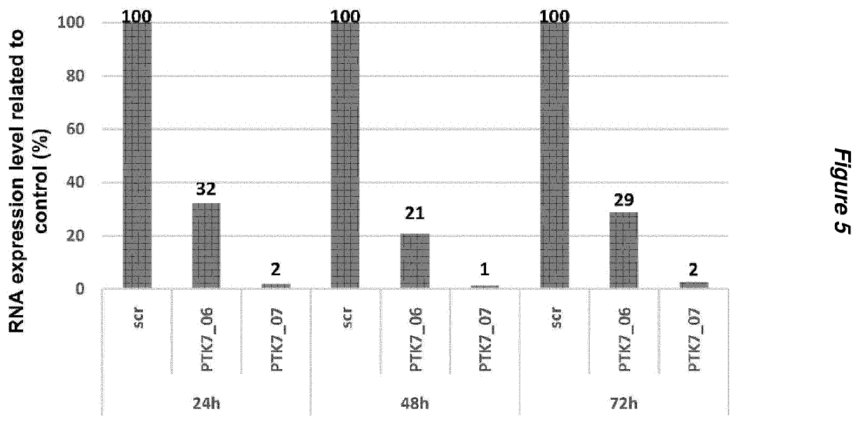

As can be seen in FIG. 5 the siRNAs targeting PTK7 (PTK7_06 and PTK7_07) were effective in reducing the mRNA levels of PTK7 at all the investigated time points following transfection.

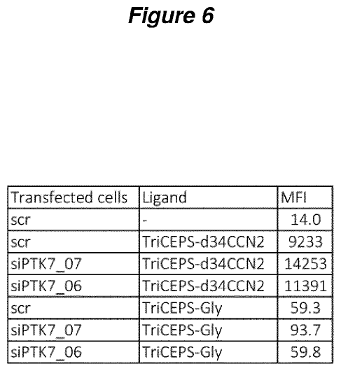

Having established the effectiveness of the PTK7 targeting siRNAs at knocking down the mRNA levels of PTK7 Rat2 fibroblasts were again transfected and 72 hours after transfection incubated with recombinant human C-terminal (domains III-IV) CCN2 coupled to the TriCEPS v.2.0 (TriCEPS-d34CCN2) at 22° C., pH7.4, 30 minutes. Following the incubation, the cells were washed, and TriCEPS-d34CCN2 labelled with streptavidin-R-PE and analysed by flow-cytometry. As can be seen from FIG. 6 the cells transfected with siRNA targeting PTK7 displayed an increase in fluorescence intensity which was most pronounced in cells transfected with the siRNA that was most effective in knocking down PTK7, “PTK7_07”.

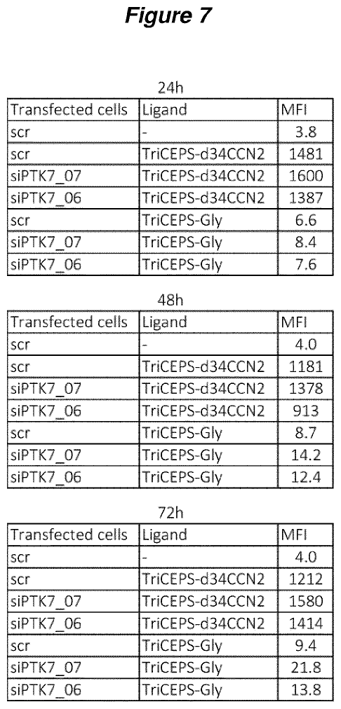

The experiment depicted in FIG. 6 was repeated at 3 different time points with binding performed at 4° C., pH6.5 for 90 minutes. Even at this low temperature, a clear shift to the right in fluorescence intensity that increased with the time since transfection could be appreciated, particularly for the cells that were targeted with the more effective siRNA PTK7-07, as shown in FIG. 7.

### Example 5: SiRNA-Phosphoprotein Assays

To assess the functional relevance of PTK7 for CCN2-induced signalling, the receptor expression in Rat2 fibroblasts was modulated by siRNAs either targeting PTK7 or not (scrambled control) and subsequently cells were stimulated with recombinant human C-terminal (domains III-IV) CCN2 and assayed for levels of pAKT S473 and GAPDH levels (for normalization).

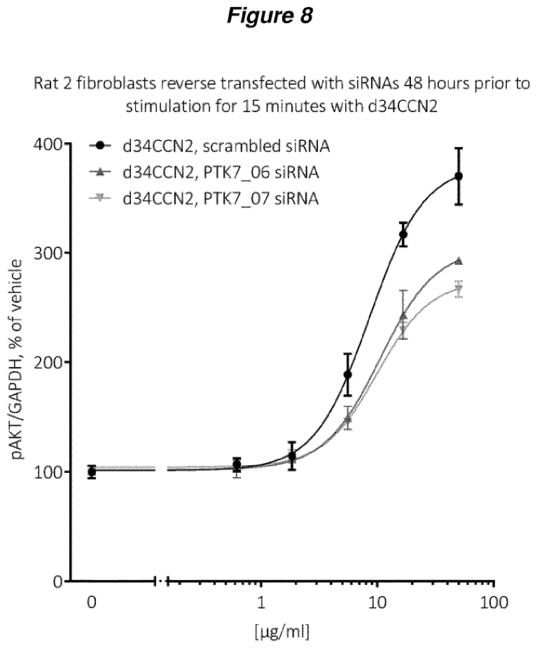

As can be seen in FIG. 8 the PTK7-targeting siRNAs PTK7_06 and PTK7_07 both reduced the concentration-dependent increase in pAKT S473/GAPDH levels observed upon stimulation with recombinant, human C-terminal (domains III-IV) CCN2.

### Example 6: SiRNA-Phosphoprotein Assays

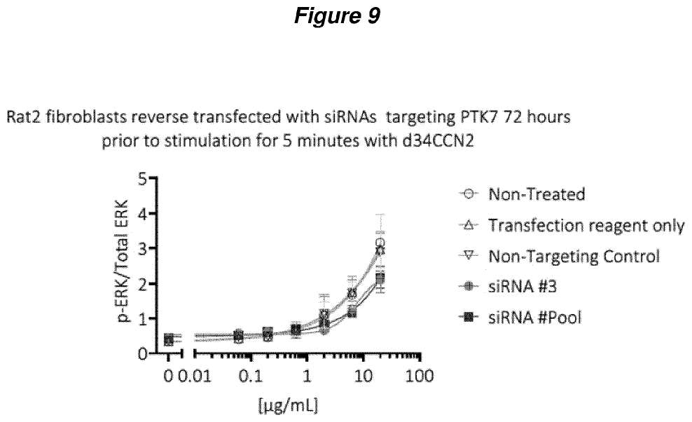

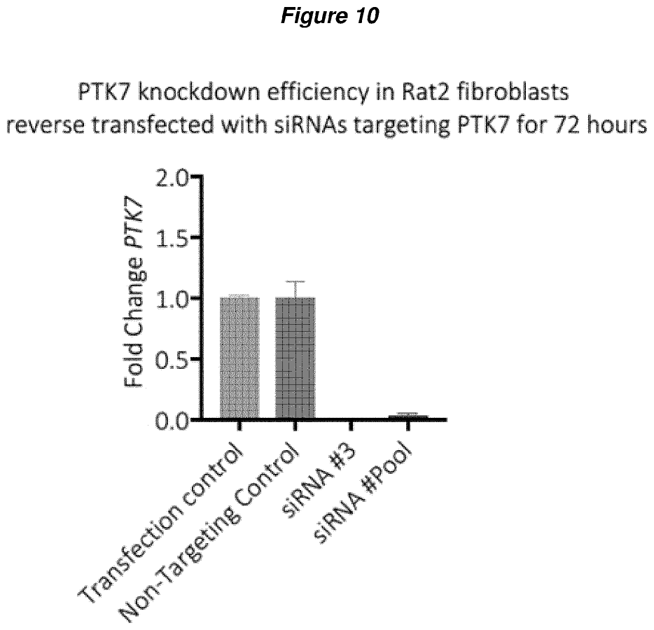

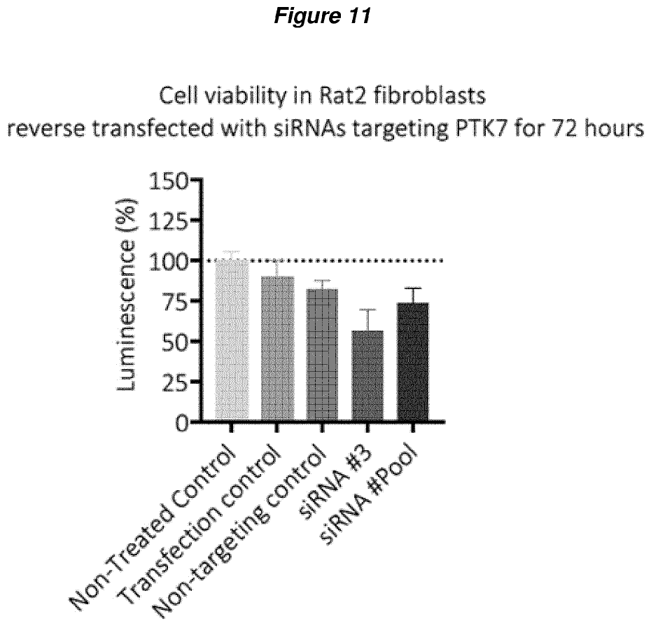

To assess the functional relevance of PTK7 for CCN2-induced signalling, the receptor expression in Rat2 fibroblasts was modulated by siRNAs either targeting PTK7 or not (non-targeting control) and subsequently cells were stimulated with recombinant human C-terminal (domains III-IV) CCN2 (“d34CCN2”) and assayed for levels of pERK/totERK. As can be seen in FIG. 9 the PTK7-targeting siRNAs “#3” and “pool” both reduced the concentration-dependent increase in pERK/totERK levels observed upon stimulation with recombinant, human C-terminal (domains III-IV) CCN2. As can be seen from FIG. 10, the knockdown efficiency was >95% and as can be seen in FIG. 11 the viability of the PTK7-targeted cells was in line with the Non-Targeting control treated cells.

### SUMMARY

Having shown that the active form of CCN2 (recombinant, human C-terminal (domains III-IV) CCN2 (“d34CCN2”) is confidently identified as binding the receptor tyrosine kinase PTK7 in two independent screens for binding partners for d34CCN2, and that the presence of PTK7 is necessary for full activation of rapid signalling responses in a d34CCN2 responsive cell line, we conclude that PTK7 is a signalling receptor for d34CCN2.

Despite PTK7 being identified in a screening system designed for picking up binding receptors, the binding of d34CCN2 to the extracellular domain of PTK7 produced with recombinant DNA technology was limited. However, the increased binding of d34CCN2 to the cell surface observed upon knockdown of PTK7 demonstrates that PTK7 affects the interaction of d34CCN2 with the cell membrane. This observed increase in d34CCN2 binding to the cell surface upon knockdown of PTK7 is likely explained via reduced internalization of d34CCN2 with reduced levels of PTK7 in the cell membrane. Binding to receptor tyrosine kinases is widely known to induce receptor-ligand complex internalization (Gho L K and Sorkin A, Cold Spring Harb Perspect Biol 2013; 5: a017459). Furthermore, the ligand-trap experiments in which extracellular domains of PTK7 reduced d34CCN2-stimulated cell signalling demonstrates that PTK7 interacts with d34CCN2 in the context of a cell culture where multiple other factors are present that are not present in an experimental setup with isolated recombinant proteins, i.e., such as the ligand-binding experiment. Thus, while it is shown that d34CCN2 binds PTK7 on cell-membranes and that PTK7 is necessary for the rapid cell signalling responses elicited by d34CCN2, it appears that additional factors, i.e., coreceptors, are necessary to increase the affinity of d34CCN2 to PTK7. Indeed, GPC1 and GPC4 identified from the binding screens are likely candidates to fill this role in that they bind d34CCN2 with a higher affinity than that observed for PTK7 and a higher affinity than previously suggested cell membrane receptors for CCN2 (Johnson et al., JASN, 28:1769-1782, 2017), while having no or limited intracellular domains and hence independent capacity to evoke an intracellular cell signalling response.

The formation of receptor complexes comprising higher affinity non-signalling coreceptors with lower affinity signalling receptors is known to occur for multiple ligands, including ligands of the receptor tyrosine kinase family (Trenker R and Jura N, Curr Opin Cell Biol. 2020 April; 63:174-185). Furthermore, GPC1 and GPC4, the identified coreceptors for d34CCN2, are previously known to serve as coreceptors also for other receptor tyrosine kinases (Filmus et al., Genome Biology 2008, Volume 9, Issue 5, Article 224). It has also previously been reported that CCN proteins can bind proteoglycans, however without specifying which ones. Here we identify the proteoglycans GPC1 and GPC4 as binders of d34CCN2 in two different cellular systems and ligand-binding assays, thus pinpointing the identities of several cell membrane proteoglycans, thereby enabling their targeting for therapeutic purposes.

The identification of CCN1 in both of the receptor binding screens fits with the paradigm that the CCNs share a signalling receptor complex, in line with the reported capacity of the recombinant human C-terminal (domains III-IV) CCN1 to elicit the same signalling and cell physiologic effects as the corresponding C-terminal fragment of CCN2 and CCN3 (Kaasbøll et al., J. Biol. Chem., 293 (46): 17953-17970, 2018).

### REFERENCES

A number of publications are cited above in order to more fully describe and disclose the invention and the state of the art to which the invention pertains. Full citations for these references are provided below.

The entirety of each of these references is incorporated herein.
1. Perbal B., J Cell Commun Signal. 2018 December; 12 (4): 625-629
2. Bradham, D M et al., J Cell Biol 114, 1285-1294 (1991)
3. Jeong D et al., JACC VOL. 67, NO. 13, 2016.
4. Xu H. et al., Clin Exp Pharmacol Physiol. 2015 November; 42 (11): 1207-19
5. Zhang L et al., Int J Mol Med. 2014 February; 33 (2): 478-86
6. Yoon P O, et al. J Mol Cell Cardiol 2010, 49:294-303
7. Brigstock, D. R. Endocr Rev 20, 189-206 (1999)
8. Kaasbøll et al. J. Biol. Chem., 293 (46): 17953-17970, 2018.
9. Ball, D. K. et al., Reproduction (2003) 125, 271-284,
10. Ball, D. K. et al., Biol Reprod 59, 828-835 (1998)
11. Ball, D. K. et al., J Endocrinol 176, R1-7 (2003)
12. Steffen, C. L. et al. Growth Factors 15, 199-213 (1998)
13. Mokalled et al. Science 354, 630-634 (2016)
14. Lau L F, J. Cell Commun. Signal. (2016) 10:121-127
15. Johnson et al. JASN, 28:1769-1782, 2017
16. Shin et al., Int J Mol Sci, 2022, 23 (4): 2391
17. H Zola. CRC Press, 1988
18. J G R Hurrell. CRC Press, 1982
19. Neuberger et al. 1988, 8th International RICMP7164916 Biotechnology Symposium Part 2, 792-799
20. Morrison et al (1984) Proc. Natl. Acad. Sd. USA 81, 6851-6855
21. Better et al (1988) Science 240, 1041
22. Skerra et al (1988) Science 240, 1038
23. Bird et al (1988) Science 242, 423
24. Huston et al (1988) Proc. Natl. Acad. Sd. USA 85, 58
25. Ward et al (1989) Nature 341, 544
26. Winter & Milstein (1991) Nature 349, 293-299.
27. Marks et al., Rio/Technology 10:779-783 (1992)
28. Barbas et al. Proc Nat. Acad. Sci. USA 91:3809-3813 (1994)
29. Schier et al. Gene 169:147-155 (1995)
30. Yelton et al. J. Immunol. 155:1994-2004 (1995)
31. Jackson et al., J. Immunol. 154 (7): 331 0-15 9 (1995)
32. Hawkins et al, J. Mol. Biol. 226:889-896 (1992)
33. Xiao Z et al., Chem. Eur. J. 2008, 14, 1769-1775
34. Myers (2003) Nature Biotechnology 21:324-328
35. Shen et al., FEBS Lett 2003 Mar. 27; 539 (1-3) 111-4
36. Barton and Medzhitov PNAS Nov. 12, 2002 vol. 99, no. 23 14943-14945
37. Wang et al., AAPS J. 2010 December; 12 (4): 492-503
38. Fire A, et al., 1998 Nature 391:806-811
39. Fire, A. Trends Genet. 15, 358-363 (1999)
40. Sharp, P. A. RNA interference 2001. Genes Dev. 15, 485-490 (2001)
41. Hammond, S. M., et al., Nature Rev. Genet. 2, 110-1119 (2001)
42. Tuschl, T. Chem. Biochem. 2, 239-245 (2001)
43. Hamilton, A. et al., Science 286, 950-952 (1999)
44. Hammond, S. M., et al., Nature 404, 293-296 (2000)
45. Zamore, P. D., et al., Cell 101, 25-33 (2000)
46. Bernstein, E., et al., Nature 409, 363-366 (2001)
47. Elbashir, S. M., et al., Genes Dev. 15, 188-200 (2001)
48. Elbashir S M, et al., 2001 Nature 411:494-498
49. Richeldi L, et al. Lancet Respir Med 2020; Sternlicht M D, et al. Respir Res 2018
50. Wang Q, et al. Fibrogenesis Tissue Repair 2011
51. Wang X, et al. Respirology 2011; Gao W, et al. PLOS One 2014
52. Zhang L, et al. Int J Mol Med 2014; Alapati D, et al. Am J Respir Cell Mol Biol
53. Daftarian N, et al. Exp Eye Res 184, 2019, 286-295
54. Bagheri A, et al. Mol Vis 2015; 21:378-390;
55. Hu B, et al. Int J Mol Sci 2014, 15, 1606-1624;
56. Yoon A, et al. PLOS One 2018, 13 (12): e0208897
57. You J J, et al., Invest Ophthalmol Vis Sci 2009 July; 50 (7): 3447-55.
58. Wallace D M, et al. Invest Ophthalmol Vis Sci 2013 December 2; 54 (13): 7836-48.
59. Dorn et al., J Mol Cell Cardiol, 121, 205-211, August 2018
60. Jeong D, et al. J Am Coll Cardiol 2016, vol. 67, No. 13, Apr. 5, 2016:1556-68
61. Bickelhaupt S, et al. J Natl Cancer Inst 2017 August 1; 109 (8).
62. Booth A J, et al., Am. J. Transplant. 10 (2010) 220-230
63. Chatzifrangkeskou M, et al. Hum Mol Genet 2016 June 1; 25 (11): 2220-2233.
64. Koshman Y E, et al. J Mol Cell Cardiol 2015, 2015 December; 89 (Pt B): 214-22
65. Morales M G, et al. Hum Mol Genet 2013 Dec. 15; 22 (24): 4938-51
66. Rebolledo D L, et al. Matrix Biol 2019 September; 82:20-37.
67. Barbe M F, et al. J Orthop Res 2019 September; 37 (9): 2004-2018
68. Adler S G, et al. Clin J Am Soc Nephrol 2010 August; 5 (8): 1420-8.
69. Dai H, et al. Ren Fail 2016 November; 38 (10): 1711-1716
70. Guha et al. FASEB J. 2007 October; 21 (12): 3355-68
71. Yokoi H et al., Kidney Int. 2008 February; 73 (4): 446-55
72. Wang Q, et al. Fibrogenesis Tissue Repair 2011 Feb. 1; 4 (1): 4.
73. Lai C F, et al. PLOS One 2013; 8 (2): e56481.
74. Qian H S, et al. PLOS One 2016 Jun. 3; 11 (6): e0156734.
75. Johnson B G, et al. J Am Soc Nephrol 2017 June; 28 (6): 1769-1782
76. Kinashi H, et al. Kidney Int 2017 October; 92 (4): 850-863.
77. Lai C F, et al. Am J Physiol Renal Physiol 2014 Sep. 1; 307 (5): F581-92
78. Okada H, et al., J. Am. Soc. Nephrol. 16 (2005) 133-143
79. Cheng O, et al., Am. J. Transplant.6 (2006) 2292-2306
80. Toda et al. (Sci Rep. 2017 Feb. 13; 7:42114)
81. Ito et al. Kidney International, Vol. 53 (1998), pp. 853-861
82. Li S, et al. Sci Rep 2016 Aug. 26; 6:32155
83. Uchio K, et al., Wound Repair Regen. 12 (2004) 60-66
84. Zhang C Y, et al. Chinese Journal of Applied Physiology, 29 (5): 411-415, 2013
85. di Mola F F, et al. Ann Surg 1999 July; 230 (1): 63-71
86. Pi L, et al. Hepatology 2015 February; 61 (2): 678-91.
87. Jensen J, et al. Plast Reconstr Surg 2018 August; 142 (2): 192e-201e.
88. Yao Y, et al. Nanomedicine 2017 November; 13 (8): 2385-2394.
89. Makino K, et al. Arthritis Res Ther 2017 Jun. 13; 19 (1): 134
90. Xu H, et al. Int J Mol Med 2015 July; 36 (1): 123-9
91. Kim J, et al. PLOS One 2018 Dec. 19; 13 (12): e0208498
92. Wei J L, et al. Arthritis Rheumatol 2018 November; 70 (11): 1745-1756.
93. Nozawa K, et al. Arthritis Rheum 2013 June; 65 (6): 1477-86.
94. Gonzalez D, et al. Hum Mol Genet 2018 Aug. 15; 27 (16): 2913-2926.
95. Song Z M, et al. Biomed Pharmacother 2019 March; 111:1429-1437
96. Aikawa T, et al. Mol Cancer Ther 2006 May; 5 (5): 1108-16
97. Dhar G, et al., Cancer Lett. 254:63-70, 2007 Aug. 28; 254 (1): 63-70.
98. Neesse A, et al. Proc Natl Acad Sci USA 2013 Jul. 23; 110 (30): 12325-30.
99. Maity G, et al. Mol Cancer Ther 2019 April; 18 (4): 788-800
100. Dornhöfer N, et al. Cancer Res 2006 Jun. 1; 66 (11): 5816-27
101. Wang M Y, et al. Cancer Res 2009 Apr. 15; 69 (8): 3482-91
102. Lin J, et al. Cancer Immunol Immunother 2012 May; 61 (5): 677-87.
103. Banerjee S, et al. Neoplasia. 5:63-73, 2003 January-February; 5 (1): 63-73.
104. Haque I, et al. Oncogene (2015) 34, 3152-3163 Jun. 11; 34 (24): 3152-63
105 Sarkar S, et al. Oncogenesis 2017 May 22; 6 (5): e340
106. Shimo T, et al. J Bone Miner Res 2006 July; 21 (7): 1045-59.
107. Kang Y, et al. Cancer Cell 2003 June; 3 (6): 537-49
108. Akalay I, et al. Oncogene 2015 Apr. 23; 34 (17): 2261-71.
109. Ono M, et al. PLOS One 2013 Aug. 14; 8 (8): e71709
110. Rho S B, et al. Biotechnol Lett 2009 January; 31 (1): 23-8.
111. Gery, S. et al. Clin. Cancer Res. 11, 7243-7254 (2005), Oct. 15; 11 (20): 7243-54.
112. Makino Y, et al. Cancer Res 2018 Sep. 1; 78 (17): 4902-4914.
113. Wang X, et al. Oncotarget 2017 Aug. 7; 8 (39): 66316-66327
114. Zeng H, et al. Cell Death Dis 2017 Jun. 15; 8 (6): e2885.
115. Tsai H C, et al. J Cell Physiol 2019 June; 234 (6): 9297-9307.
116. Tsai H C, et al. PLOS One 2014 Mar. 17; 9 (3): e90159.
117. Lu H, et al. Ann Hematol 2014 March; 93 (3): 485-492.
118. Finger E C, et al. Oncogene 2014 Feb. 27; 33 (9): 1093-100
119. Ohara Y, et al. Oncotarget 2018 Apr. 6; 9 (26): 18494-18509
120. Tanaka S, et al. Oncogene 2001 Sep. 6; 20 (39): 5525-32
121. Ji J, et al., Br J Cancer. 2015; 113 (6): 921-933)
122. Sun S, et al. Cell Biol Int 2020 April; 44 (4): 998-1008.
123. Chai D M, et al. J Exp Clin Cancer Res 2019 Feb. 26; 38 (1): 102.
124. Frewer K A, et al. Cancer Genomics Proteomics 2013 July-August; 10 (4): 187-96.
125. Dwivedi N, et al., 2020, 31 (8): 1697-1710
126. Vainio et al. JACC: Basic to Translation Science. Vol. 4, No. 1, 219 February 2019:83-84
127. N. Daftarian et al. Experimental Eye Research 208 (2021) 108622
128. Petrosino J M et al., FASEB J, VOL 33, February 2019.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An agent that binds to PTK7 and has PTK7 antagonist activity for use in a method of treatment or prophylaxis of fibrosis, metabolic disease, inflammatory disease, autoimmune disease, retinal disease, muscular dystrophy, cardiac disease, or polycystic kidney disease.

2. The agent for use according to claim 1, wherein the agent:
(i) inhibits CCN ligand binding to PTK7;
(ii) inhibits PTK7-dependent signalling transduction;
(iii) inhibits homodimerization with PTK7 and/or heterodimerization with an RTK receptor;
(iv) inhibits PTK7 associating with a GPC; and/or
(v) promotes PTK7 internalisation, optionally wherein the agent promotes PTK7 internalisation and degradation.

3. The agent for use according to claim 1 or claim 2, wherein the agent is selected from the group consisting of an antibody, an antibody-like molecule, an aptamer, and a bifunctional molecule comprising a PTK7 binding moiety and a cell-surface lysosome shuttling receptor binding moiety.

4. An agent that inhibits expression of PTK7 for use in a method of treatment or prophylaxis of fibrosis, metabolic disease, inflammatory disease, autoimmune disease, retinal disease, muscular dystrophy, cardiac disease, or polycystic kidney disease.

5. The agent for use according to claim 4, wherein the agent:
(i) inhibits transcription of the gene encoding PTK7;
(ii) inhibits post-transcriptional processing of RNA encoding PTK7;
(iii) destabilises RNA encoding PTK7; and/or
(iv) promotes the degradation of RNA encoding PTK7.

6. The agent for use according to claim 4 or claim 5, wherein the agent is selected from the group consisting of an siRNA, a shRNA, an miRNA, an antisense oligonucleotide, and an RNA-guided endonuclease system.

7. An agent comprising a PTK7 polypeptide or fragment thereof for use in a method of treatment or prophylaxis of fibrosis, metabolic disease, inflammatory disease, autoimmune disease, retinal disease, muscular dystrophy, cardiac disease, or polycystic kidney disease, wherein said PTK7 polypeptide or fragment thereof binds to a CCN ligand and inhibits binding of said CCN ligand to a transmembrane PTK7 receptor and/or a membrane bound GPC.

8. The agent for use according to claim 7, wherein the agent comprises a heterologous moiety.

9. The agent for use according to claim 8, wherein the heterologous moiety increases the stability of the agent and/or increases the serum half-life of the agent.

10. An agent that binds to a GPC and inhibits GPC-CCN ligand-PTK7 complex formation for use in a method of treatment or prophylaxis of fibrosis, metabolic disease, inflammatory disease, autoimmune disease, cancer, retinal disease, muscular dystrophy, cardiac disease, or polycystic kidney disease.

11. The agent for use according to claim 10, wherein the agent:
(i) binds to a GPC and/or a GPC complex comprising a CCN ligand and a GPC;
(ii) binds to a GPC and inhibits CCN ligand binding to said GPC;
(iii) binds to a GPC and inhibits the GPC from associating with and/or binding to PTK7;
(iv) inhibits PTK7-dependent signalling transduction; and/or
(v) promotes GPC internalisation and degradation.

12. The agent for use according to claim 10 or claim 11, wherein the agent is selected from the group consisting of an antibody, an antibody-like molecule, an aptamer, and a bifunctional molecule comprising a GPC binding moiety and a cell-surface lysosome shuttling receptor binding moiety.

13. An agent that inhibits expression of a GPC for use in a method of treatment or prophylaxis of fibrosis, metabolic disease, inflammatory disease, autoimmune disease, cancer, retinal disease, muscular dystrophy, cardiac disease, or polycystic kidney disease.

14. The agent for use according to claim 13, wherein the agent:
(i) inhibits transcription of the gene encoding a GPC;
(ii) inhibits post-transcriptional processing of RNA encoding a GPC;
(iii) destabilises RNA encoding a GPC; and/or
(iv) promotes degradation of RNA encoding a GPC.

15. The agent for use according to claim 13 or claim 14, wherein the agent is selected from the group consisting of an siRNA, a shRNA, an miRNA, an antisense oligonucleotide, and an RNA-guided endonuclease system.

16. An agent comprising a GPC polypeptide or fragment thereof for use in a method of treatment or prophylaxis of fibrosis, metabolic disease, inflammatory disease, autoimmune disease, cancer, retinal disease, muscular dystrophy, cardiac disease, or polycystic kidney disease, wherein said GPC polypeptide or fragment thereof binds a CCN ligand and inhibits binding of said CCN ligand to a transmembrane PTK7 receptor and/or a membrane bound GPC.

17. The agent for use according to claim 16, wherein the agent comprises a heterologous moiety.

18. The agent for use according to claim 17, wherein the heterologous moiety increases the stability of the agent and/or increases the serum half-life of the agent.

19. The agent for use according to any one of claims 10 to 18, wherein the GPC is GPC1, GPC2, GPC3, GPC4, GPC5, or GPC6.

20. The agent according to claim 19, wherein the GPC is GPC1 or GPC4.

21. The agent for use according to any one of claims to 10 to 20, wherein said cancer is selected from the group consisting of pancreatic cancer, breast cancer, prostate cancer, cervical cancer, ovarian cancer, liver cancer, bladder cancer, brain cancer, bone cancer, blood cancer, melanoma, stomach cancer, mouth cancer, oesophageal cancer, colorectal cancer, or lung cancer.

22. The agent for use according to any one of claims 1 to 21, wherein the agent inhibits CCN ligand-induced signalling.

23. The agent for use according to any one of claims 1 to 20 or claim 22, wherein the metabolic disease is diabetes.

24. The agent for use according to any one of claims 1 to 20 or claim 22, wherein the inflammatory disease or autoimmune disease is rheumatoid arthritis or inflammatory bowel disease.

25. The agent for use according to any one of claims 1 to 20 or claim 22, wherein the retinal disease is diabetic retinopathy or age-related macular degeneration.

26. The agent for use according to any one of claims 1 to 20 or claim 22, wherein the muscular dystrophy is Duchenne Muscular dystrophy (DMD).

27. A pharmaceutical composition comprising the agent for use according to any one of claims 1 to 26.

28. The pharmaceutical composition according to claim 27, wherein the pharmaceutical composition comprises a pharmaceutically acceptable excipient.

29. A method of screening for an agent that binds to PTK7 and has PTK7 antagonist activity, wherein the agent inhibits CCN ligand-induced signalling, comprising:
(i) providing a PTK7 or a fragment thereof;
(ii) contacting said PTK7 or fragment thereof with one or more candidate agents:
(iii) determining whether the one or more candidate agents bind to said PTK7 or fragment thereof;
(iv) selecting a candidate agent that binds to said PTK7 or fragment thereof from said one or more candidate agents;
(v) providing a cell expressing PTK7;
(vi) contacting said cell with the candidate agent that binds to a PTK7 or a fragment thereof; and
(vii) determining whether the candidate agent inhibits CCN ligand-induced signalling.

30. A method of screening for an agent that inhibits expression of PTK7, wherein the agent inhibits CCN ligand-induced signalling, comprising:
(i) providing a cell expressing PTK7;
(ii) contacting said cell with one or more candidate agents;
(iii) determining whether said one or more candidate agents inhibit expression of PTK7;
(iv) selecting a candidate agent that inhibits expression of PTK7 from said one or more candidate agents;
(v) contacting a cell expressing PTK7 with the candidate agent that inhibits expression of PTK7; and
(vi) determining whether the candidate agent inhibits CCN ligand-induced signalling.

31. A method of screening for an agent that binds to a GPC and inhibits GPC-CCN ligand-PTK7 complex formation is provided, wherein the agent inhibits CCN ligand-induced signalling, comprising:
(i) providing a GPC or fragment thereof;
(ii) contacting said GPC or fragment thereof with one or more candidate agents:
(iii) determining whether the one or more candidate agents bind to said GPC or fragment thereof;
(iv) selecting a candidate agent that binds to said GPC or fragment thereof from said one or more candidate agents;
(v) providing a cell expressing a GPC;
(vi) contacting said cell with the candidate agent that binds to a GPC or a fragment thereof; and
(vii) determining whether the candidate agent inhibits CCN ligand-induced signalling.

32. A method of screening for an agent that inhibits expression of a GPC, wherein the agent inhibits CCN ligand-induced signalling, comprising:
(i) providing a cell expressing a GPC;
(ii) contacting said cell with one or more candidate agents;
(iii) determining whether said one or more candidate agents inhibit expression of a GPC;
(iv) selecting a candidate agent that inhibits expression of a GPC from said one or more candidate agents;
(v) contacting a cell expressing a GPC with the candidate agent that inhibits expression of a GPC; and
(vi) determining whether the candidate agent inhibits CCN ligand-induced signalling.


SEQUENCE LISTING (32 sequences)

SEQ ID NO: 1 - protein, 1070 aa, Homo sapiens
MGAARGSPARPRRLPLLSVLLLPLLGGTQTAIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 2 - protein, 704 aa, Homo sapiens
MGAARGSPARPRRLPLLSVLLLPLLGGTQTAIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQT

SEQ ID NO: 3 - protein, 558 aa, Homo sapiens
MELRARGWWLLCAAAALVACARGDPASKSRSCGEVRQIYGAKGFSLSDVPQAEISGEHLRICPQGYTCCTSEMEENLANRSHAELETALRDSSRVLQAMLATQLRSFDDHFQHLLNDSERTLQATFPGAFGELYTQNARAFRDLYSELRLYYRGANLHLEETLAEFWARLLERLFKQLHPQLLLPDDYLDCLGKQAEALRPFGEAPRELRLRATRAFVAARSFVQGLGVASDVVRKVAQVPLGPECSRAVMKLVYCAHCLGVPGARPCPDYCRNVLKGCLANQADLDAEWRNLLDSMVLITDKFWGTSGVESVIGSVHTWLAEAINALQDNRDTLTAKVIQGCGNPKVNPQGPGPEEKRRRGKLAPRERPPSGTLEKLVSEAKAQLRDVQDFWISLPGTLCSEKMALSTASDDRCWNGMARGRYLPEVMGDGLANQINNPEVEVDITKPDMTIRQQIMQLKIMTNRLRSAYNGNDVDFQDASDDGSGSGSGDGCLDDLCSRKVSRKSSSSRTPLTHALPGLSEQEGQKTSAASCPQPPTFLLPLLLFLALTVARPRWR

SEQ ID NO: 4 - protein, 579 aa, Homo sapiens
MSALRPLLLLLLPLCPGPGPGPGSEAKVTRSCAETRQVLGARGYSLNLIPPALISGEHLRVCPQEYTCCSSETEQRLIRETEATFRGLVEDSGSFLVHTLAARHRKFDEFFLEMLSVAQHSLTQLFSHSYGRLYAQHALIFNGLFSRLRDFYGESGEGLDDTLADFWAQLLERVFPLLHPQYSFPPDYLLCLSRLASSTDGSLQPFGDSPRRLRLQITRTLVAARAFVQGLETGRNVVSEALKVPVSEGCSQALMRLIGCPLCRGVPSLMPCQGFCLNVVRGCLSSRGLEPDWGNYLDGLLILADKLQGPFSFELTAESIGVKISEGLMYLQENSAKVSAQVFQECGPPDPVPARNRRAPPPREEAGRLWSMVTEEERPTTAAGTNLHRLVWELRERLARMRGFWARLSLTVCGDSRMAADASLEAAPCWTGAGRGRYLPPVVGGSPAEQVNNPELKVDASGPDVPTRRRRLQLRAATARMKTAALGHDLDGQDADEDASGSGGGQQYADDWMAGAVAPPARPPRPPYPPRRDGSGGKGGGGSARYNQGRSRSGGASIGFHTQTILILSLSALALLGPR

SEQ ID NO: 5 - protein, 580 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 6 - protein, 556 aa, Homo sapiens
MARFGLPALLCTLAVLSAALLAAELKSKSCSEVRRLYVSKGFNKNDAPLHEINGDHLKICPQGSTCCSQEMEEKYSLQSKDDFKSVVSEQCNHLQAVFASRYKKFDEFFKELLENAEKSLNDMFVKTYGHLYMQNSELFKDLFVELKRYYVVGNVNLEEMLNDFWARLLERMFRLVNSQYHFTDEYLECVSKYTEQLKPFGDVPRKLKLQVTRAFVAARTFAQGLAVAGDVVSKVSVVNPTAQCTHALLKMIYCSHCRGLVTVKPCYNYCSNIMRGCLANQGDLDFEWNNFIDAMLMVAERLEGPFNIESVMDPIDVKISDAIMNMQDNSVQVSQKVFQGCGPPKPLPAGRISRSISESAFSARFRPHHPEERPTTAAGTSLDRLVTDVKEKLKQAKKFWSSLPSNVCNDERMAAGNGNEDDCWNGKGKSRYLFAVTGNGLANQGNNPEVQVDTSKPDILILRQIMALRVMTSKMKNAYNGNDVDFFDISDESSGEGSGSGCEYQQCPSEFDYNATDHAGKSANEKADSAGVRPGAQAYLLTVFCILFLVMQREWR

SEQ ID NO: 7 - protein, 572 aa, Homo sapiens
MDAQTWPVGFRCLLLLALVGSARSEGVQTCEEVRKLFQWRLLGAVRGLPDSPRAGPDLQVCISKKPTCCTRKMEERYQIAARQDMQQFLQTSSSTLKFLISRNAAAFQETLETLIKQAENYTSILFCSTYRNMALEAAASVQEFFTDVGLYLFGADVNPEEFVNRFFDSLFPLVYNHLINPGVTDSSLEYSECIRMARRDVSPFGNIPQRVMGQMGRSLLPSRTFLQALNLGIEVINTTDYLHFSKECSRALLKMQYCPHCQGLALTKPCMGYCLNVMRGCLAHMAELNPHWHAYIRSLEELSDAMHGTYDIGHVLLNFHLLVNDAVLQAHLNGQKLLEQVNRICGRPVRTPTQSPRCSFDQSKEKHGMKTTTRNSEETLANRRKEFINSLRLYRSFYGGLADQLCANELAAADGLPCWNGEDIVKSYTQRVVGNGIKAQSGNPEVKVKGIDPVINQIIDKLKHVVQLLQGRSPKPDKWELLQLGSGGGMVEQVSGDCDDEDGCGGSGSGEVKRTLKITDWMPDDMNFSDVKQIHQTDTGSTLDTTGAGCAVATESMTFTLISVVMLLPGIW

SEQ ID NO: 8 - protein, 555 aa, Homo sapiens
MPSWIGAVILPLLGLLLSLPAGADVKARSCGEVRQAYGAKGFSLADIPYQEIAGEHLRICPQEYTCCTTEMEDKLSQQSKLEFENLVEETSHFVRTTFVSRHKKFDEFFRELLENAEKSLNDMFVRTYGMLYMQNSEVFQDLFTELKRYYTGGNVNLEEMLNDFWARLLERMFQLINPQYHFSEDYLECVSKYTDQLKPFGDVPRKLKIQVTRAFIAARTFVQGLTVGREVANRVSKVSPTPGCIRALMKMLYCPYCRGLPTVRPCNNYCLNVMKGCLANQADLDTEWNLFIDAMLLVAERLEGPFNIESVMDPIDVKISEAIMNMQENSMQVSAKVFQGCGQPKPAPALRSARSAPENFNTRFRPYNPEERPTTAAGTSLDRLVTDIKEKLKLSKKVWSALPYTICKDESVTAGTSNEEECWNGHSKARYLPEIMNDGLTNQINNPEVDVDITRPDTFIRQQIMALRVMTNKLKNAYNGNDVNFQDTSDESSGSGSGSGCMDDVCPTEFEFVTTEAPAVDPDRREVDSSAAQRGHSLLSWSLTCIVLALQRLCR

SEQ ID NO: 9 - RNA, 4222 nt, synthetic construct
  source 1..4222: mol_type = other RNA
  mRNA 1..4222: note = mRNA
actgggcgcgcgcggctccggctcgggacgcctcgggacgcctcggggtcgggctccggctgcggctgctgctgcggcgcccgcgctccggtgcgctccgcctcctgtgcccgccgcggagcgcagtctgcgcgcccgccgtgcgccctcagctccttttcctgagcccgccgcgatgggagctgcgcggggatccccggccagaccccgccggttgcctctgctcagcgtcctgctgctgccgctgctgggcggtacccagacagccattgtcttcatcaagcagccgtcctcccaggatgcactgcaggggcgccgggcgctgcttcgctgtgaggttgaggctccgggcccggtacatgtgtactggctgctcgatggggcccctgtccaggacacggagcggcgtttcgcccagggcagcagcctgagctttgcagctgtggaccggctgcaggactctggcaccttccagtgtgtggctcgggatgatgtcactggagaagaagcccgcagtgccaacgcctccttcaacatcaaatggattgaggcaggtcctgtggtcctgaagcatccagcctcggaagctgagatccagccacagacccaggtcacacttcgttgccacattgatgggcaccctcggcccacctaccaatggttccgagatgggacccccctttctgatggtcagagcaaccacacagtcagcagcaaggagcggaacctgacgctccggccagctggtcctgagcatagtgggctgtattcctgctgcgcccacagtgcttttggccaggcttgcagcagccagaacttcaccttgagcattgctgatgaaagctttgccagggtggtgctggcaccccaggacgtggtagtagcgaggtatgaggaggccatgttccattgccagttctcagcccagccacccccgagcctgcagtggctctttgaggatgagactcccatcactaaccgcagtcgccccccacacctccgcagagccacagtgtttgccaacgggtctctgctgctgacccaggtccggccacgcaatgcagggatctaccgctgcattggccaggggcagaggggcccacccatcatcctggaagccacacttcacctagcagagattgaagacatgccgctatttgagccacgggtgtttacagctggcagcgaggagcgtgtgacctgccttccccccaagggtctgccagagcccagcgtgtggtgggagcacgcgggagtccggctgcccacccatggcagggtctaccagaagggccacgagctggtgttggccaatattgctgaaagtgatgctggtgtctacacctgccacgcggccaacctggctggtcagcggagacaggatgtcaacatcactgtggccactgtgccctcctggctgaagaagccccaagacagccagctggaggagggcaaacccggctacttggattgcctgacccaggccacaccaaaacctacagttgtctggtacagaaaccagatgctcatctcagaggactcacggttcgaggtcttcaagaatgggaccttgcgcatcaacagcgtggaggtgtatgatgggacatggtaccgttgtatgagcagcaccccagccggcagcatcgaggcgcaagcccgtgtccaagtgctggaaaagctcaagttcacaccaccaccccagccacagcagtgcatggagtttgacaaggaggccacggtgccctgttcagccacaggccgagagaagcccactattaagtgggaacgggcagatgggagcagcctcccagagtgggtgacagacaacgctgggaccctgcattttgcccgggtgactcgagatgacgctggcaactacacttgcattgcctccaacgggccgcagggccagattcgtgcccatgtccagctcactgtggcagtttttatcaccttcaaagtggaaccagagcgtacgactgtgtaccagggccacacagccctactgcagtgcgaggcccagggggaccccaagccgctgattcagtggaaaggcaaggaccgcatcctggaccccaccaagctgggacccaggatgcacatcttccagaatggctccctggtgatccatgacgtggcccctgaggactcaggccgctacacctgcattgcaggcaacagctgcaacatcaagcacacggaggcccccctctatgtcgtggacaagcctgtgccggaggagtcggagggccctggcagccctcccccctacaagatgatccagaccattgggttgtcggtgggtgccgctgtggcctacatcattgccgtgctgggcctcatgttctactgcaagaagcgctgcaaagccaagcggctgcagaagcagcccgagggcgaggagccagagatggaatgcctcaacggtgggcctttgcagaacgggcagccctcagcagagatccaagaagaagtggccttgaccagcttgggctccggccccgcggccaccaacaaacgccacagcacaagtgataagatgcacttcccacggtctagcctgcagcccatcaccacgctggggaagagtgagtttggggaggtgttcctggcaaaggctcagggcttggaggagggagtggcagagaccctggtacttgtgaagagcctgcagagcaaggatgagcagcagcagctggacttccggagggagttggagatgtttgggaagctgaaccacgccaacgtggtgcggctcctggggctgtgccgggaggctgagccccactacatggtgctggaatatgtggatctgggagacctcaagcagttcctgaggatttccaagagcaaggatgaaaaattgaagtcacagcccctcagcaccaagcagaaggtggccctatgcacccaggtagccctgggcatggagcacctgtccaacaaccgctttgtgcataaggacttggctgcgcgtaactgcctggtcagtgcccagagacaagtgaaggtgtctgccctgggcctcagcaaggatgtgtacaacagtgagtactaccacttccgccaggcctgggtgccgctgcgctggatgtcccccgaggccatcctggagggtgacttctctaccaagtctgatgtctgggccttcggtgtgctgatgtgggaagtgtttacacatggagagatgccccatggtgggcaggcagatgatgaagtactggcagatttgcaggctgggaaggctagacttcctcagcccgagggctgcccttccaaactctatcggctgatgcagcgctgctgggccctcagccccaaggaccggccctccttcagtgagattgccagcgccctgggagacagcaccgtggacagcaagccgtgaggagggagcccgctcaggatggcctgggcaggggaggacatctctagagggaagctcacagcatgatgggcaagatccctgtcctcctgggccctgaggcccctgccctagtgcaacaggcattgctgaggtctgagcagggcctggcctttcctcctcttcctcaccctcatcctttgggaggctgacttggacccaaactgggcgactagggctttgagctgggcagttttccctgccacctcttcctctatcagggacagtgtgggtgccacaggtaaccccaatttctggccttcaacttctccccttgaccgggtccaactctgccactcatctgccaactttgcctggggagggctaggcttgggatgagctgggtttgtggggagttccttaatattctcaagttctgggcacacagggttaatgagtctcttggcccactggtcccacttgggggtctagaccaggattatagaggacacagcaagtgagtcctccccactctgggcttgtgcacactgacccagacccacgtcttccccacccttctctcctttcctcatcctaagtgcctggcagatgaaggagttttcaggagcttttgacactatataaaccgccctttttgtatgcaccacgggcggcttttatatgtaattgcagcgtggggtgggtgggcatgggaggtaggggtgggccctggagatgaggagggtgggccatccttaccccacacttttattgttgtcgttttttgtttgttttgtttttttgtttttgtttttgtttttacactcgctgctctcaataaataagccttttttacaa

SEQ ID NO: 10 - RNA, 3719 nt, synthetic construct
  source 1..3719: mol_type = other RNA
  mRNA 1..3719: note = mRNA
gccgagccgggactgcgctagcccgccgcgctctgggctgcccgagcgagcgttcggacctcgcaccccgcgcgccccgcgccgccgccgccgccggcttttgttgtctccgcctcctcggccgccgccgcctctggaccgcgagccgcgcgcgccgggaccttggctctgcccttcgcgggcgggaactgcgcaggacccggccaggatccgagagaggcgcgggcgggtggccgggggcgccgccggccccgccatggagctccgggcccgaggctggtggctgctatgtgcggccgcagcgctggtcgcctgcgcccgcggggacccggccagcaagagccggagctgcggcgaggtccgccagatctacggagccaagggcttcagcctgagcgacgtgccccaggcggagatctcgggtgagcacctgcggatctgtccccagggctacacctgctgcaccagcgagatggaggagaacctggccaaccgcagccatgccgagctggagaccgcgctccgggacagcagccgcgtcctgcaggccatgcttgccacccagctgcgcagcttcgatgaccacttccagcacctgctgaacgactcggagcggacgctgcaggccaccttccccggcgccttcggagagctgtacacgcagaacgcgagggccttccgggacctgtactcagagctgcgcctgtactaccgcggtgccaacctgcacctggaggagacgctggccgagttctgggcccgcctgctcgagcgcctcttcaagcagctgcacccccagctgctgctgcctgatgactacctggactgcctgggcaagcaggccgaggcgctgcggcccttcggggaggccccgagagagctgcgcctgcgggccacccgtgccttcgtggctgctcgctcctttgtgcagggcctgggcgtggccagcgacgtggtccggaaagtggctcaggtccccctgggcccggagtgctcgagagctgtcatgaagctggtctactgtgctcactgcctgggagtccccggcgccaggccctgccctgactattgccgaaatgtgctcaagggctgccttgccaaccaggccgacctggacgccgagtggaggaacctcctggactccatggtgctcatcaccgacaagttctggggtacatcgggtgtggagagtgtcatcggcagcgtgcacacgtggctggcggaggccatcaacgccctccaggacaacagggacacgctcacggccaaggtcatccagggctgcgggaaccccaaggtcaacccccagggccccgggcctgaggagaagcggcgccggggcaagctggccccgcgggagaggccaccttcaggcacgctggagaagctggtctccgaagccaaggcccagctccgcgacgtccaggacttctggatcagcctcccagggacactgtgcagtgagaagatggccctgagcactgccagtgatgaccgctgctggaacgggatggccagaggccggtacctccccgaggtcatgggtgacggcctggccaaccagatcaacaaccccgaggtggaggtggacatcaccaagccggacatgaccatccggcagcagatcatgcagctgaagatcatgaccaaccggctgcgcagcgcctacaacggcaacgacgtggacttccaggacgccagtgacgacggcagcggctcgggcagcggtgatggctgtctggatgacctctgcagccggaaggtcagcaggaagagctccagctcccggacgcccttgacccatgccctcccaggcctgtcagagcaggaaggacagaagacctcggctgccagctgcccccagcccccgaccttcctcctgcccctcctcctcttcctggcccttacagtagccaggccccggtggcggtaactgccccaaggccccagggacagaggccaaggactgactttgccaaaaatacaacacagacgatatttaattcacctcagcctggagaggcctggggtgggacagggagggccggcggctctgagcaggggcaggcgcagaggtcccagccccaggcctggcctcgcctgcctttctgccttttaattttgtatgaggtcctcaggtcagctgggagccagtgtgcccaaaagccatgtatttcagggacctcaggggcacctccggctgcctagccctccccccagctccctgcaccgccgcagaagcagcccctcgaggcctacagaggaggcctcaaagcaacccgctggagcccacagcgagcctgtgccttcctccccgcctcctcccactgggactcccagcagagcccaccagccagccctggcccaccccccagcctccagagaagccccgcacgggctgtctgggtgtccgccatccagggtctggcagagcctctgagatgatgcatgatgccctcccctcagcgcaggctgcagagcccggccccacctccctgcgcccttgaggggccccagcgtctgcagggtgacgcctgagacagcaccactgctgaggagtctgaggactgtcctcccacagacctgcagtgaggggccctccatgcgcagatgaggggccactgacccacctgcgcttctgctggaggaggggaagctgggcccaaaggcccagggaggcagcgtgggctctgccaatgtgggctgcccctcgcacacagggctcacagggcaggccttgctggggtccagggctgttggaggaccccgagggctgaggagcagccaggacccgcctgctcccatcctcacccagatcaggaaccagggcctccctgttcacggtgacacaggtcagggctcagagtgaccctcagctgtcacctgctcacagggatgctggtggctggtgagaccccgcactgcagacgggaatgcctaggtcccttcccgacccagccagctgcagggcacggggacctggatagttaagggcttttccaaacatgcatccatttactgacacttcctgtccttgttcatggagagctgttcgctcctcccagatggcttcggagggccgcagggcccaccttggaccctggtgacctcctgtcactcactgaggccatcagggccctgccccaggcctggacgggccctccttccctcctgtgccccagctgccaggcggccctggggaggggtggtgtggtgttgggaaggggtcctgcagggggaggaggacttggagggtctgggggcagctgtcctgaaccgactgaccctgaggaggccgcttagtgctgctttgcttttcatcaccgtcccgcacagtggacggaggtccccggttgctggtcaggtccccatggcttgttctctggaacctgactttagatgttttgggatcaggagcccccaacacaggcaagtccaccccataataaccctgccagtgccagggtgggctggggactctggcacagtgatgccgggcgccaggacagcagcactcccgctgcacacagacggcctaggggtggcgctcagaccccaccctacgctcatctctggaaggggcagccctgagtggtcactggtcagggcagtggccaagcctgctgtgtccttcctccacaaggtccccccaccgctcagtgtcagcgggtgacgtgtgttcttttgagtccttgtatgaataaaaggctggaaaccta

SEQ ID NO: 11 - RNA, 2546 nt, synthetic construct
  source 1..2546: mol_type = other RNA
  mRNA 1..2546: note = mRNA
gctcccattgtctcggcagatgccgcctggtccagctatcgtgctcggtattcagttttccggagcagcgctctttctctggcccgcggagcggtcccgcggccgagtaccggattcccgagtttgggaggctctgctttcctccttaggacccactttgccgtcctggggtggctgcagttatgtccgcgctgcgacctctcctgcttctgctgctgcctctgtgtcccggtcctggtcccggacccgggagcgaggcaaaggtcacccggagttgtgcagagacccggcaggtgctgggggcccggggatatagcttaaacctaatccctcccgccctgatctcaggtgagcacctccgggtctgtccccaggagtacacctgctgttccagtgagacagagcagaggctgatcagggagactgaggccaccttccgaggcctggtggaggacagcggctcctttctggttcacacactggctgccaggcacagaaaatttgatgagttttttctggagatgctctcagtagcccagcactctctgacccagctcttctcccactcctacggccgcctgtatgcccagcacgccctcatattcaatggcctgttctctcggctgcgagacttctatggggaatctggtgaggggttggatgacaccctggcggatttctgggcacagctcctggagagagtgttcccgctgctgcacccacagtacagcttcccccctgactacctgctctgcctctcacgcttggcctcatctaccgatggctctctgcagccctttggggactcaccccgccgcctccgcctgcagataacccggaccctggtggctgcccgagcctttgtgcagggcctggagactggaagaaatgtggtcagcgaagcgcttaaggtgccggtgtctgaaggctgcagccaggctctgatgcgtctcatcggctgtcccctgtgccggggggtcccctcacttatgccctgccagggcttctgcctcaacgtggttcgtggctgtctcagcagcaggggactggagcctgactggggcaactatctggatggtctcctgatcctggctgataagctccagggccccttttcctttgagctgacggccgagtccattggggtgaagatctcggagggtttgatgtacctgcaggaaaacagtgcgaaggtgtccgcccaggtgtttcaggagtgcggcccccccgacccggtgcctgcccgcaaccgtcgagccccgccgccccgggaagaggcgggccggctgtggtcgatggtgaccgaggaggagcggcccacgacggccgcaggcaccaacctgcaccggctggtgtgggagctccgcgagcgtctggcccggatgcggggcttctgggcccggctgtccctgacggtgtgcggagactctcgcatggcagcggacgcctcgctggaggcggcgccctgctggaccggagccgggcggggccggtacttgccgccagtggtcgggggctccccggccgagcaggtcaacaaccccgagctcaaggtggacgcctcgggccccgatgtcccgacacggcggcgtcggctacagctccgggcggccacggccagaatgaaaacggccgcactgggacacgacctggacgggcaggacgcggatgaggatgccagcggctctggagggggacagcagtatgcagatgactggatggctggggctgtggctcccccagcccggcctcctcggcctccataccctcctagaagggatggttctgggggcaaaggaggaggtggcagtgcccgctacaaccagggccggagcaggagtgggggggcatctattggttttcacacccaaaccatcctcattctctccctctcagccctggccctgcttggacctcgataacgggggaggggtgccctagcatcagaagggttcatggccctttcccctcctcccccctcagctgggcctggggaggagtcgaagggggctgcagagagggtagagaagggactttgcaggtgaatggctggggccccaaatccaggagattttcatcagaggtgggtgggtgttcacaatatttattttttcatttggtaatgggaggggggcctgggggtatttatttaggagggagtgtggtttccttagaaggtatagtctctagccctctaaggctggggctggtgatcagccccaacagagaaaatgaggagtttagagttgcagctggggaaggggtttgaaggaagttggaagtggggaggggtgggggcatctggtctcagaaatggaccagctggatgcagggcaggggactgagggtgcttgagtaggatgtgagacttcatgggcctgggttctgttgagttttttcagtatcaatttcttaaaccaaattttaaaaaaaacaaggtgggggggtgctcatctcgtgacctctgccacccacatccttcacaaactccatgtttcagtgtttgagtccatgtttattctgcaaataaatggtaatgtattgga

SEQ ID NO: 12 - RNA, 2336 nt, synthetic construct
  source 1..2336: mol_type = other RNA
  mRNA 1..2336: note = mRNA
aggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcaggatggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactgaaactgagaagaaaatatggcacttcaaatatcctatcttcttcctgtgtatagggctagacttacagattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactgactgcctggtgcccagcacatgtgctgccctacagcaccctgtggtcttcctcgataaagggaaccactttcttatttttttctatttttttttttttgttatcctgtatacctcctccagccatgaagtagaggactaaccatgtgttatgttttcgaaaatcaaatggtatcttttggaggaagatacattttagtggtagcatatagattgtccttttgcaaagaaagaaaaaaaaccatcaagttgtgccaaattattctcctatgtttggctgctagaacatggttaccatgtctttctctctcactccctccctttctatcgttctctctttgcatggatttctttgaaaaaaaataaattgctcaaataaaaa

SEQ ID NO: 13 - protein, 603 aa, synthetic construct
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTETEKKIWHFKYPIFFLCIGLDLQIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 14 - RNA, 2267 nt, synthetic construct
  source 1..2267: mol_type = other RNA
  mRNA 1..2267: note = mRNA
aggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcaggatggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactgactgcctggtgcccagcacatgtgctgccctacagcaccctgtggtcttcctcgataaagggaaccactttcttatttttttctatttttttttttttgttatcctgtatacctcctccagccatgaagtagaggactaaccatgtgttatgttttcgaaaatcaaatggtatcttttggaggaagatacattttagtggtagcatatagattgtccttttgcaaagaaagaaaaaaaaccatcaagttgtgccaaattattctcctatgtttggctgctagaacatggttaccatgtctttctctctcactccctccctttctatcgttctctctttgcatggatttctttgaaaaaaaataaattgctcaaataaaaa

SEQ ID NO: 15 - RNA, 4959 nt, synthetic construct
  source 1..4959: mol_type = other RNA
  mRNA 1..4959: note = mRNA
agtcaatcagcggcagagctgagaagggcacagccgcgcggcggagggcagagtcagccgagccgagtccagccggacgagcggaccagcgcagggcagcccaagcagcgcgcagcgaacgcccgccgccgcccacaccctctgcggtccccgcggcgcctgccacccttccctccttccccgcgtccccgcctcgccggccagtcagcttgccgggttcgctgccccgcgaaaccccgaggtcaccagcccgcgcctctgcttccctgggccgcgcgccgcctccacgccctccttctcccctggcccggcgcctggcaccggggaccgttgcctgacgcgaggcccagctctacttttcgccccgcgtctcctccgcctgctcgcctcttccaccaactccaactccttctccctccagctccactcgctagtccccgactccgccagccctcggcccgctgccgtagcgccgcttcccgtccggtcccaaaggtgggaacgcgtccgccccggcccgcaccatggcacggttcggcttgcccgcgcttctctgcaccctggcagtgctcagcgccgcgctgctggctgccgagctcaagtcgaaaagttgctcggaagtgcgacgtctttacgtgtccaaaggcttcaacaagaacgatgcccccctccacgagatcaacggtgatcatttgaagatctgtccccagggttctacctgctgctctcaagagatggaggagaagtacagcctgcaaagtaaagatgatttcaaaagtgtggtcagcgaacagtgcaatcatttgcaagctgtctttgcttcacgttacaagaagtttgatgaattcttcaaagaactacttgaaaatgcagagaaatccctgaatgatatgtttgtgaagacatatggccatttatacatgcaaaattctgagctatttaaagatctcttcgtagagttgaaacgttactacgtggtgggaaatgtgaacctggaagaaatgctaaatgacttctgggctcgcctcctggagcggatgttccgcctggtgaactcccagtaccactttacagatgagtatctggaatgtgtgagcaagtatacggagcagctgaagcccttcggagatgtccctcgcaaattgaagctccaggttactcgtgcttttgtagcagcccgtactttcgctcaaggcttagcggttgcgggagatgtcgtgagcaaggtctccgtggtaaaccccacagcccagtgtacccatgccctgttgaagatgatctactgctcccactgccggggtctcgtgactgtgaagccatgttacaactactgctcaaacatcatgagaggctgtttggccaaccaaggggatctcgattttgaatggaacaatttcatagatgctatgctgatggtggcagagaggctagagggtcctttcaacattgaatcggtcatggatcccatcgatgtgaagatttctgatgctattatgaacatgcaggataatagtgttcaagtgtctcagaaggttttccagggatgtggaccccccaagcccctcccagctggacgaatttctcgttccatctctgaaagtgccttcagtgctcgcttcagaccacatcaccccgaggaacgcccaaccacagcagctggcactagtttggaccgactggttactgatgtcaaggagaaactgaaacaggccaagaaattctggtcctcccttccgagcaacgtttgcaacgatgagaggatggctgcaggaaacggcaatgaggatgactgttggaatgggaaaggcaaaagcaggtacctgtttgcagtgacaggaaatggattagccaaccagggcaacaacccagaggtccaggttgacaccagcaaaccagacatactgatccttcgtcaaatcatggctcttcgagtgatgaccagcaagatgaagaatgcatacaatgggaacgacgtggacttctttgatatcagtgatgaaagtagtggagaaggaagtggaagtggctgtgagtatcagcagtgcccttcagagtttgactacaatgccactgaccatgctgggaagagtgccaatgagaaagccgacagtgctggtgtccgtcctggggcacaggcctacctcctcactgtcttctgcatcttgttcctggttatgcagagagagtggagataattctcaaactctgagaaaaagtgttcatcaaaaagttaaaaggcaccagttatcacttttctaccatcctagtgactttgctttttaaatgaatggacaacaatgtacagtttttactatgtggccactggtttaagaagtgctgactttgttttctcattcagttttgggaggaaaagggactgtgcattgagttggttcctgctcccccaaaccatgttaaacgtggctaacagtgtaggtacagaactatagttagttgtgcatttgtgattttatcactctattatttgtttgtatgtttttttctcatttcgtttgtgggtttttttttccaactgtgatctcgccttgtttcttacaagcaaaccagggtcccttcttggcacgtaacatgtacgtatttctgaaatattaaatagctgtacagaagcaggttttatttatcatgttatcttattaaaagaaaaagcccaaaaagcagtaaaatttccatttctccctgttattttagttgccttatctggagagacgtggaggtgattttcttttttttaaattattattaagacagaatgtgagggcacaagcaggcttctgagccacttgtcagattgtattcaaagcatcaatccaagaaggaggttatgtgtacttcatttattggtgatagttggaagagactgcagactactgctttgaatgagttgaattacataagctaagatcactataggtccatttcttgaacccacttatacataaaatgtaacccatttagaaaaagattctggatatcatcccccttgaaagatagaaagcattcaggatgtcccagttatcacatgttcacacttgggtttaggggtgtttttttttaaaaccaggcaggttagctagcccaccctgtgctagttttcatgttcacactgaccctatttgaattaatatcctttgttagagtggtcgagatttcaaacccaattatgtacagggagctgtctgagagctagccagaactggggtacagcctgggctcagggaatagctgtcaacactcgggcaaagtttttgtctgtgcatgtgtatctccatttgttttgggatcccagtttttgttttaagagagtataaggtgtctcatttgagtctttttcttacctagccccctcttatcagtaaaacaaaggacttgccatggttcacagcaatgtgctacgatccaagatatcagccaaggagcccacttaggggagaactaggtgtccagatttttgtatgtgttgtttttcttgggggatggggtggggtgggagtaggtagagctgagaatactacatcttagtggtgacctttagccacgtgggtgaagtggcaaaggccatggccatatctgttgtcccaggccaaagactaacaactgccttgggaatcccttccttgtgtccttaccaaatgatagctcataaaactctgataatgtaacaaatcactttcaaaggagttcccagaagtcttcagaaagactaaaattctgtctcttcctgctttagacagccattaagatcccaactaattttaccgaacctaaaacccacaaagaggttgtttgtgttattgttcaatcttcagttgtaagagtaattctctatttttatattgaaacataattacttgatagctcagggtctacatttcattcaactttttacaccaaattctgcagagtggtcaaaatggaatattgggggctgttgtaaacagaggcttaattttattagaagtagccagttatttattaaagcatgatgttaataaaataggcatattctggctggtgtgtataagtggttttttttttaaaacaaatcagaagacataaaattgaaaaggcactccatttatatataagggaaaatagattgtaaatagctaaggggccttgtgagaacttggaaaatgtgctgatacttagtaactacctcagttgtgaggaatttcctttttagcttagactgctattacttttacttggtagaaatgaatctggtctccaaaatatggctcagagagttatccccatagaccttgttagattgtttttaatttaaaaccttgaactctgcccagctgttttcttccagtcaaattctctaaaccaataacattttccaaattaagtattcttgaaggttcttctgaactcttactgatttgttttctttctttgcattttttcctgttactcttttattatttaaattgtctcttgcttttgaacttcaactatgaaataaacaaatcatagatttttccattcaggtctgactggataactcttggcttctaagcagctggcaagctcacccccactccccaaaaaagaagaaaggcaagttgaatattttaaaaccttaagagtttggagtatgaagtagaataaaagccttgttaggagaagacctgacttttccaaagttaacagcatgtgaagggcttgtgttttacaaagtcatttataaattatttttctttaaaacacatcacctactctgtaccttaaagattttacaactcttagatttatattttatttcaaaaatcgtaactagcacctgttattttgattaaaagacactgcgaagaattgacgtaaatttctaaagtttagttactgagtgttcaaaacctaacagttcacatgaagtaaaaattgtcatttgtgattacagaatcaaaataaaaataggtatgaatggtgcta

SEQ ID NO: 16 - RNA, 2943 nt, synthetic construct
  source 1..2943: mol_type = other RNA
  mRNA 1..2943: note = mRNA
atcccaggttagctgctgcgagccgagccgggcggcggaggcggcggcggcggcggcagtggcggcagtggcggcagtggcggcagcggcagcagttgcagcagtggtggccagagcggatgcttgcgggctccctgcggctccactagttttcttcgccccgcccagccgcccactcttctcggctagggaagaagaccagagggtgctcagctggaaaactctggtgtctcagcttagggcctcctccgggaagagctaactgctcccaggtgaagccggtgcccgcgggcggtccgtacaccccgcagccggctcgcaccgctcgagagcctcggccgctgtgtcttccacgtctgcagctcagccagggcgcgcagggcgagtggggtccactggcgggtaaaggggaccaggacggcgaggatggacgcacagacctggcccgtgggctttcgctgcctcctccttctggccctggttgggtccgcccgcagcgagggcgtgcagacctgcgaagaagttcggaaacttttccagtggcggctgctgggagctgtcagggggctgccggattcgccgcgggcaggacctgatcttcaggtttgcatatccaaaaagcctacatgttgcaccaggaagatggaggagagatatcagattgcggctcgccaggatatgcagcagtttcttcaaacgtccagctctacattaaagtttctaatatctcgaaatgcggctgcttttcaagaaacccttgaaactctcatcaaacaagcagaaaattacaccagtatacttttttgcagtacctacaggaacatggccttggaggctgctgcttcggttcaggagttcttcactgatgtggggctgtatttatttggtgcggatgttaatcctgaagaatttgtaaacagattttttgacagtctttttcctctggtctacaaccacctcattaaccctggtgtgactgacagttccctggaatactcagaatgcatccggatggctcgccgggatgtgagtccatttggtaatattccccaaagagtaatgggacagatggggaggtccctgctgcccagccgcacttttctgcaggcactcaatctgggcattgaagtcatcaacaccacagactatctgcacttctccaaagagtgcagcagagccctcctgaagatgcaatactgcccgcactgccaaggcctggcgctcactaagccttgtatgggatactgcctcaatgtcatgcgaggctgcctggcgcacatggcggagcttaatccacactggcatgcatatatccggtcgttggaagaactctcggatgcaatgcatggaacatacgacattggacacgtgctgctgaactttcacttgcttgttaatgatgctgtgttacaggctcacctcaatggacaaaaattattggaacaggtaaataggatttgtggccgccctgtaagaacacccacacaaagcccccgttgttcttttgatcagagcaaagagaagcatggaatgaagaccaccacaaggaacagtgaagagacgcttgccaacagaagaaaagaatttatcaacagccttcgactgtacaggtcattctatggaggtctagctgatcagctttgtgctaatgaattagctgctgcagatggacttccctgctggaatggagaagatatagtaaaaagttatactcagcgtgtggttggaaatggaatcaaagcccagtctggaaatcctgaagtcaaagtcaaaggaattgatcctgtgataaatcagattattgataaactgaagcatgttgttcagttgttacagggtagatcacccaaacctgacaagtgggaacttcttcagctgggcagtggtggaggcatggttgaacaagtcagtggggactgtgatgatgaagatggttgcgggggatcaggaagtggagaagtcaagaggacactgaagatcacagactggatgccagatgatatgaacttcagtgatgtaaagcaaatccatcaaacagacactggcagtactttagacacaacaggagcaggatgtgcagtggcgactgaatctatgacattcactctgataagtgtggtgatgttacttcccgggatttggtaactgaactcttctgtcctgacataccttactgaagtctcgatttcttctctctctgcatatgcctggaataagagatcctttttcaatgtaacaattatatttatgaaaagatatgttacactaacttctcagaagccaagctgaaatattcataaagtccctaaaactcaacgtttaaatgacacactttaaaaatatgtcttttttcaatctaactgaaaaccttcttaacttctaatatattaaatctgaagatgtgaagggcacagaagtgactttgaataagaagaatttagtgtatctgtaattttattatcaattccaagccccttcctttctaaattaaaaatgttttcatttgaaagtgtatttgccagacaatgaaaacagtatgcagtatttcttaaagtattgaaattagaatatcatgaaataaatcaaaacatacaatggcaagtagtatgcatgcatattcaagagactcttccatttttgcaagctgtagaaggaaatgtctgaatgtctataagttatggggtagattcttgagaagcatttcatataatttcactgaagaaccttgataattttgacccactgtaacttagccactgatgaaccttaaagctgagtattttattaacacctgatttgtattccattatattcaaaatgcatctttggtattgtgcctctgctcccatctctctctttgcctcatagatttagctatgttgggaagcacatgcttgctctaggaatatctccaataaagctgttaactatttggtggaaaaa

SEQ ID NO: 17 - RNA, 7121 nt, synthetic construct
  source 1..7121: mol_type = other RNA
  mRNA 1..7121: note = mRNA
cctttctccttccctcttgcctccagtgactgtctccaggatttctctcttcctatttcaggaggactctcacaggctcccacagcctgtgttaagctgaggtttcccctagatctcgtatatccccaacacatacctccacgcacacacatccccaagaacctcgagctcacaccaacagacacacgcgcgcatacacactcgctctcgcttgtccatctccctcccgggggagccggcgcgcgctcccacctttgccgcacactccggcgagccgagcccgcagcgctccaggattctgcggctcggaactcggattgcagctctgaacccccatggtggttttttaaacacttcttttccttctcttcctcgttttgattgcaccgtttccatctgggggctagaggagcaaggcagcagccttcccagccagcccttgttggcttgccatcgtccatctggcttataaaagtttgctgagcgcagtccagagggctgcgctgctcgtcccctcggctggcagaagggggtgacgctgggcagcggcgaggagcgcgccgctgcctctggcgggctttcggcttgaggggcaaggtgaagagcgcaccggccgtggggtttaccgagctggatttgtatgttgcaccatgccttcttggatcggggctgtgattcttcccctcttggggctgctgctctccctccccgccggggcggatgtgaaggctcggagctgcggagaggtccgccaggcgtacggtgccaagggattcagcctggcggacatcccctaccaggagatcgcaggggaacacttaagaatctgtcctcaggaatatacatgctgcaccacagaaatggaagacaagttaagccaacaaagcaaactcgaatttgaaaaccttgtggaagagacaagccattttgtgcgcaccacttttgtgtccaggcataagaaatttgacgaatttttccgagagctcctggagaatgcagaaaagtcactaaatgatatgtttgtacggacctatggcatgctgtacatgcagaattcagaagtcttccaggacctcttcacagagctgaaaaggtactacactgggggtaatgtgaatctggaggaaatgctcaatgacttttgggctcggctcctggaacggatgtttcagctgataaaccctcagtatcacttcagtgaagactacctggaatgtgtgagcaaatacactgaccagctcaagccatttggagacgtgccccggaaactgaagattcaggttacccgcgccttcattgctgccaggacctttgtccaggggctgactgtgggcagagaagttgcaaaccgagtttccaaggtcagcccaaccccagggtgtatccgtgccctcatgaagatgctgtactgcccatactgtcgggggcttcccactgtgaggccctgcaacaactactgtctcaacgtcatgaagggctgcttggcaaatcaggctgacctcgacacagagtggaatctgtttatagatgcaatgctcttggtggcagagcgactggaggggccattcaacattgagtcggtcatggacccgatagatgtcaagatttctgaagccattatgaacatgcaagaaaacagcatgcaggtgtctgcaaaggtctttcagggatgtggtcagcccaaacctgctccagccctcagatctgcccgctcagctcctgaaaattttaatacacgtttcaggccctacaatcctgaggaaagaccaacaactgctgcaggcacaagcttggaccggctggtcacagacataaaagagaaattgaagctctctaaaaaggtctggtcagcattaccctacactatctgcaaggacgagagcgtgacagcgggcacgtccaacgaggaggaatgctggaacgggcacagcaaagccagatacttgcctgagatcatgaatgatgggctcaccaaccagatcaacaatcccgaggtggatgtggacatcactcggcctgacactttcatcagacagcagattatggctctccgtgtgatgaccaacaaactaaaaaacgcctacaatggcaatgatgtcaatttccaggacacaagtgatgaatccagtggctcagggagtggcagtgggtgcatggatgacgtgtgtcccacggagtttgagtttgtcaccacagaggcccccgcagtggatcccgaccggagagaggtggactcttctgcagcccagcgtggccactccctgctctcctggtctctcacctgcattgtcctggcactgcagagactgtgcagataatcttgggtttttggtcagatgaaactgcattttagctatctgaatggccaactcacttcttttcttacactcttggacaatggaccatgccacaaaaacttaccgttttctatgagaagagagcagtaatgcaatctgcctccctttttgttttcccaaagagtaccgggtgccagactgaactgcttcctctttccttcagctatctgtggggaccttgtttattctagagagaattcttactcaaatttttcgtaccaggagattttcttaccttcatttgcttttatgctgcagaagtaaaggaatctcacgttgtgagggttttttttttctcatttaaaataaaaaaggaagaaagaaaataattttccttgtaaaatcgggccaaaccccaagacagctacattttcaacaaaaaagcaaacagagaaaaataaatgaactttaacactgtaagttcagcattgacagccaacttccagtgtaagcttttttgtgacaaatccgggtttaaaaatgcttatggggagaaagcttaaagtttgttctaaatgtagtgaaaatgcactgttgtcttggaggtatcatcttgcactctaaccaaacaacaccaaattaatctggaggtactgatactaaatttggaatcccgtctttaatatgagaacacaaattactacaactcagtacctctgtcttactctgcccaatttcaatttagtaatggcaccattattcttaattcccttaatgtctgtccgctgtatccaattggcacagattttccccttcatcttcaattactagtagctacccccatcaattcaccttcctcaagagtctcaattgaaaataatctttgatgatacaatctctgcttttgccacaggtttgacattaactagacgtttctgcccctgtgctcattaccctcccaaaacaatagtttttttaatgaattgaaacacaattagatccattcctgagttttcatttttaactatttactttgtgtcagttggtttctgcatcacaagggcattctcttacaaaacaactcacaacagaaaaaaaaagtcaagacaaaaaatatatatatatagtattgacatggagatttctttcacttttttagtcttagtatgatcatctatttttatatctatgtatatataaatcacagattttaacttcttaattccaagctcagggttgacacacctttgtaagaaaatgttttgtcaaccagctcttcttgttttgtgctgctcaaagaagggattcctcagtgatcggtgagcaccaagaatcaagaaagagaactttttatgtatctaactgtccatttattaaaaatttgccagggcacacccttctgcatgagtgtgcttcagcctgggtgctccagactacaccaaactcatgggatgagccattggtgtgcagggctttaccggtgatggaaactgtatgtgtaaagaatgttattcatgaaaaaccattgctttcatgtaaaacaccttcggcctaggattcctgcctggcacttggatacattacctaactcaataggccagtttagagccacagcctaacctgttcaccaagtaggccaaagccaggcctttcgctccccctgacagagagaagcaaaataaacccaactgggggctttatgaatacagttggctatccatatgtttcttagtttttatgatgcatctggagtagggaaaagtaatgtttcaagacaccatagtaggccagttacattttaattaactcatcgacagtattgacacgtaaatggtatttttcaatctgttctcatcaaaattaaaattagttattgatttagtcatcacatttaagtagaacacatgtggcacaatagtattaagtatatttgagcacttttgcaagacagaaagtatttacaagcatctattgctactttatgcctaatgaaatataaaatgtaggatgatgagagactttgtggcattctttcatttaaacctttatgaaattaaccatctagttcatcattacatactcctagaaaaccatccatttcactgcccacattttggcttcctaccacaaattccacataagacttctctatgaaggaccagtcatttacattgctcatttcattctgtgttcagaagtcagtattgtctttctccaataattaaggtgcaatacaaattaaatcaaccttgattttccagactatttgaataataacttgcttttatggctttaaactccaggtctttcctttttaaaatatttctgcattctcttttatgaactagatcaggatgccttgtttttcctgtgctttcacccccaaaaggttttgtgcatgtgtatgaatacacataatattagtgaatgtgaacccatgttctatacgcactaaacacacagttaatatataaacgcctgcttcatttgtttcaacaccttcattatcaatatgattcatattgatgagatatagtgatgcataatgagccaggcagagagctgaaatgatatcagttaatgatgtcacagtgttagcaccctgcatggatgtggtcaaaatatcattcaagttgtacttcctagtttttatcatcatactactatgtcatatgattttcagactaatttaacatgagaaatgttttggccctaaaggatttcacttaacctagttaccctatttcagtaagtattttggaattcaaccctcgaattattttttctcatttcagcatagtgataggggatgcaatgaggcttcattattttttatgacctgcccctcatttgctctgatgttccctaaattctgtaatcatatcataacttttgttatgaatagagaggaatgggctcactgaaacctgacactagaaattggtgggtgatgctcataactgcaaacacttagcttattgaagtgcctctatttacatgttctttagttataatatgtatttttctaacagaaatacacgtctgtaattggtatatattatactttgtatgtgtcacaacaaaagctaaacagaggctaaagtctttagcagagaagaatgaattacaaatttaccaataagagctctgtttatggttctgtattgaaggacaaccatgacaaccttctgtcattttcacgaaggccaagttggtactttgattatagccatcgtagaacattagcactagaaatctgtctcccttacaatgacttacctagctagcatcaagtaacttgtatcacctgctttaaatgtaagggatagaaaatactattttgtctacactgaaaaacattattattcattgataagtagtaataattataaaagtaacattctttaaagcaaacacattgaaaatattattacaggcagaaccctgatttttaatttatattccctgctgcagacattagccttctttaattcttttgccattacatacatgtttcggctacagactgaacacgaactaaagtccctaaggaggaaaaaaccttaacatattcctgagatgtgattcttttattacttctttttgcaaagctaattatcaaatgatttgattaaacctagacgcattaggaaatgcaagttttacctaaaacttgcagaaatttaaacacatttccatttacttactaacgaacagacctgatttttatttaaaaacacaatttagctttgccaacagaagagtaaaggaaaccataacttataaatttgtaatgtgttcctccttgttggagacattagccagtgtttagttttagaccaacttatacaaagagagtattgatttgtacacattctggagacttgctatatcatggtttgctatggcacagactcagcttagcatggggacattttgaacttcattcaaagttgagtagttactggaacctttgacattgccttgtaatgaggtacttccaaaaaaagacccctaacaatggcataatagtgaggtctctctgtgcatatacataatatatatgcaggataaatgtgtgcaccaatcaaatctaaaattttatgtgactgtcaaatgaatattatttgggttaagatttttcatttctgatttggatagaaaattgctattaatcttgtattaaaatagtttttaaaaatctaccagtgccctttctgcattttcttaattattgtcatagtctaaatttcagagccatggaattttttgtgtttgagacatttttagaagcttcaatattaaaaaacaacaacaaacctcgtatattaaaacagtgccactctcctaagtacactcagcatctgaaatcaggaactatttgaactttagaacatttagagcttcccaaatgtgtatggaaaatactgtagcgctgttcttttgtactgatttcttctcagaggataaatttagtgacttgaaaactgatgtcaatactttataaatgaggtcagaaaataagatcaaatgtgaaaagactaataatttcccaatttagccaattgcattgatttttatacttctgaaatgtttccatcctctcaggcatgctaaatattgtataacctgctaaagatttatttcacaaatgcttattgaactcttattctctataaaccctgctctaggggatttatcactggctgtgaaaaaccaccagttgaaaattattcattccttaatgcagctaatcataattattacagataaataatgtatttcaagaatgatcttataaataaaggaacccatacaaacaaa

SEQ ID NO: 18 - protein, 674 aa, synthetic construct
AIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQT

SEQ ID NO: 19 - protein, 507 aa, synthetic construct
DPASKSRSCGEVRQIYGAKGFSLSDVPQAEISGEHLRICPQGYTCCTSEMEENLANRSHAELETALRDSSRVLQAMLATQLRSFDDHFQHLLNDSERTLQATFPGAFGELYTQNARAFRDLYSELRLYYRGANLHLEETLAEFWARLLERLFKQLHPQLLLPDDYLDCLGKQAEALRPFGEAPRELRLRATRAFVAARSFVQGLGVASDVVRKVAQVPLGPECSRAVMKLVYCAHCLGVPGARPCPDYCRNVLKGCLANQADLDAEWRNLLDSMVLITDKFWGTSGVESVIGSVHTWLAEAINALQDNRDTLTAKVIQGCGNPKVNPQGPGPEEKRRRGKLAPRERPPSGTLEKLVSEAKAQLRDVQDFWISLPGTLCSEKMALSTASDDRCWNGMARGRYLPEVMGDGLANQINNPEVEVDITKPDMTIRQQIMQLKIMTNRLRSAYNGNDVDFQDASDDGSGSGSGDGCLDDLCSRKVSRKSSSSRTPLTHALPGLSEQEGQKTS

SEQ ID NO: 20 - protein, 237 aa, synthetic construct
IEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 911 aa, synthetic construct
AIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 513 aa, synthetic construct
DPASKSRSCGEVRQIYGAKGFSLSDVPQAEISGEHLRICPQGYTCCTSEMEENLANRSHAELETALRDSSRVLQAMLATQLRSFDDHFQHLLNDSERTLQATFPGAFGELYTQNARAFRDLYSELRLYYRGANLHLEETLAEFWARLLERLFKQLHPQLLLPDDYLDCLGKQAEALRPFGEAPRELRLRATRAFVAARSFVQGLGVASDVVRKVAQVPLGPECSRAVMKLVYCAHCLGVPGARPCPDYCRNVLKGCLANQADLDAEWRNLLDSMVLITDKFWGTSGVESVIGSVHTWLAEAINALQDNRDTLTAKVIQGCGNPKVNPQGPGPEEKRRRGKLAPRERPPSGTLEKLVSEAKAQLRDVQDFWISLPGTLCSEKMALSTASDDRCWNGMARGRYLPEVMGDGLANQINNPEVEVDITKPDMTIRQQIMQLKIMTNRLRSAYNGNDVDFQDASDDGSGSGSGDGCLDDLCSRKVSRKSSSSRTPLTHALPGLSEQEGQKTSHHHHHH

SEQ ID NO: 23 - protein, 1040 aa, synthetic construct
AIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 24 - protein, 507 aa, synthetic construct; identical to SEQ ID NO: 19

SEQ ID NO: 25 - protein, 531 aa, synthetic construct
SEAKVTRSCAETRQVLGARGYSLNLIPPALISGEHLRVCPQEYTCCSSETEQRLIRETEATFRGLVEDSGSFLVHTLAARHRKFDEFFLEMLSVAQHSLTQLFSHSYGRLYAQHALIFNGLFSRLRDFYGESGEGLDDTLADFWAQLLERVFPLLHPQYSFPPDYLLCLSRLASSTDGSLQPFGDSPRRLRLQITRTLVAARAFVQGLETGRNVVSEALKVPVSEGCSQALMRLIGCPLCRGVPSLMPCQGFCLNVVRGCLSSRGLEPDWGNYLDGLLILADKLQGPFSFELTAESIGVKISEGLMYLQENSAKVSAQVFQECGPPDPVPARNRRAPPPREEAGRLWSMVTEEERPTTAAGTNLHRLVWELRERLARMRGFWARLSLTVCGDSRMAADASLEAAPCWTGAGRGRYLPPVVGGSPAEQVNNPELKVDASGPDVPTRRRRLQLRAATARMKTAALGHDLDGQDADEDASGSGGGQQYADDWMAGAVAPPARPPRPPYPPRRDGSGGKGGGGSARYNQGRSRSG

SEQ ID NO: 26 - protein, 530 aa, synthetic construct
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHN

SEQ ID NO: 27 - protein, 511 aa, synthetic construct
ALLAAELKSKSCSEVRRLYVSKGFNKNDAPLHEINGDHLKICPQGSTCCSQEMEEKYSLQSKDDFKSVVSEQCNHLQAVFASRYKKFDEFFKELLENAEKSLNDMFVKTYGHLYMQNSELFKDLFVELKRYYVVGNVNLEEMLNDFWARLLERMFRLVNSQYHFTDEYLECVSKYTEQLKPFGDVPRKLKLQVTRAFVAARTFAQGLAVAGDVVSKVSVVNPTAQCTHALLKMIYCSHCRGLVTVKPCYNYCSNIMRGCLANQGDLDFEWNNFIDAMLMVAERLEGPFNIESVMDPIDVKISDAIMNMQDNSVQVSQKVFQGCGPPKPLPAGRISRSISESAFSARFRPHHPEERPTTAAGTSLDRLVTDVKEKLKQAKKFWSSLPSNVCNDERMAAGNGNEDDCWNGKGKSRYLFAVTGNGLANQGNNPEVQVDTSKPDILILRQIMALRVMTSKMKNAYNGNDVDFFDISDESSGEGSGSGCEYQQCPSEFDYNATDHAGKSANEKADS

SEQ ID NO: 28 - protein, 532 aa, synthetic construct
EGVQTCEEVRKLFQWRLLGAVRGLPDSPRAGPDLQVCISKKPTCCTRKMEERYQIAARQDMQQFLQTSSSTLKFLISRNAAAFQETLETLIKQAENYTSILFCSTYRNMALEAAASVQEFFTDVGLYLFGADVNPEEFVNRFFDSLFPLVYNHLINPGVTDSSLEYSECIRMARRDVSPFGNIPQRVMGQMGRSLLPSRTFLQALNLGIEVINTTDYLHFSKECSRALLKMQYCPHCQGLALTKPCMGYCLNVMRGCLAHMAELNPHWHAYIRSLEELSDAMHGTYDIGHVLLNFHLLVNDAVLQAHLNGQKLLEQVNRICGRPVRTPTQSPRCSFDQSKEKHGMKTTTRNSEETLANRRKEFINSLRLYRSFYGGLADQLCANELAAADGLPCWNGEDIVKSYTQRVVGNGIKAQSGNPEVKVKGIDPVINQIIDKLKHVVQLLQGRSPKPDKWELLQLGSGGGMVEQVSGDCDDEDGCGGSGSGEVKRTLKITDWMPDDMNFSDVKQIHQTDTGSTLDTTGAGCAVATES

SEQ ID NO: 29 - protein, 506 aa, synthetic construct
DVKARSCGEVRQAYGAKGFSLADIPYQEIAGEHLRICPQEYTCCTTEMEDKLSQQSKLEFENLVEETSHFVRTTFVSRHKKFDEFFRELLENAEKSLNDMFVRTYGMLYMQNSEVFQDLFTELKRYYTGGNVNLEEMLNDFWARLLERMFQLINPQYHFSEDYLECVSKYTDQLKPFGDVPRKLKIQVTRAFIAARTFVQGLTVGREVANRVSKVSPTPGCIRALMKMLYCPYCRGLPTVRPCNNYCLNVMKGCLANQADLDTEWNLFIDAMLLVAERLEGPFNIESVMDPIDVKISEAIMNMQENSMQVSAKVFQGCGQPKPAPALRSARSAPENFNTRFRPYNPEERPTTAAGTSLDRLVTDIKEKLKLSKKVWSALPYTICKDESVTAGTSNEEECWNGHSKARYLPEIMNDGLTNQINNPEVDVDITRPDTFIRQQIMALRVMTNKLKNAYNGNDVNFQDTSDESSGSGSGSGCMDDVCPTEFEFVTTEAPAVDPDRREVDS

SEQ ID NO: 30 - protein, 7 aa, synthetic construct
PKKKRKV

SEQ ID NO: 31 - DNA, 40 bp, synthetic construct
  source 1..40: mol_type = other DNA
gggcgcgtagatgacgagcagtcctaacatcgtttaggac

SEQ ID NO: 32 - DNA, 41 bp, synthetic construct
  source 1..41: mol_type = other DNA
atctaactgctgcgccgccgggaaaatactgtacggttaga